(19) United States
(12) Patent Application Publication
(10) Pub. No.: US 2023/0227921 A1
(43) Pub. Date: Jul. 20, 2023

(54) Methods and Compositions for Detecting Virulent and Avirulent Escherichia coli Strains

(71) Applicant: The Florida State University Research Foundation, Inc., Tallahassee, FL (US)

(72) Inventor: Prashant Singh, Tallahassee, FL (US)

(21) Appl. No.: 18/157,434

(22) Filed: Jan. 20, 2023

Related U.S. Application Data
(60) Provisional application No. 63/301,236, filed on Jan. 20, 2022.

(51) Int. Cl. C12Q 1/689; C12Q 1/686; C12Q 1/6818

(52) U.S. Cl. CPC C12Q 1/689; C12Q 1/686; C12Q 1/6818; C12Q 2600/142

(57) ABSTRACT

Disclosed herein are methods for detecting virulent Shiga toxin-producing E. coli (STEC) strains O26, O103, O121, and O111 in a biological sample comprising the steps of: (i) enriching the bacterial concentration of the biological sample to result in an enriched sample; (ii) isolating DNA from said enriched biological sample; and (iii) detecting virulent strain in said isolated DNA sample via real-time PCR and a melt curve assay. Also disclosed are primers for said assay, as well as kits comprising said primers.

### PRIORITY STATEMENT

This application claims benefit of U.S. Provisional Application No. 63/301,236, filed Jan. 20, 2023, incorporated herein by reference in its entirety.

### BACKGROUND

Beef, a major industry, is considered important agricultural commerce in the United States. The US beef agribusiness, at present, is valued at $69 billion (USDA, 2019). As of January 2020, 94.4 million cattle heads were processed, producing approximately 27 billion pounds of beef products (USDA, ERS, 2020). Foodborne pathogens are a common cause of concern for beef products, resulting in costly product recalls. Shiga toxin-producing Escherichia coli (STEC) is one of the top foodborne pathogens commonly associated with beef products, and cattle are considered the primary reservoirs for STEC strains. These STEC strains are broadly divided into O157 and non-O157 STEC (USDA, 2020). The top STEC serogroups O157, O26, O121, O103, O145, O45, and O111, are considered adulterants in non-intact beef. Members of these seven STEC serogroups can be further divided into virulent and avirulent strains. The virulent strains of STEC serogroups harbor the stx1, stx2, and eae virulence genes and are pathogenic to humans. These virulent STEC strains can cause different symptoms ranging from bloody diarrhea to renal failure. While the avirulent STEC strains lack significant virulence genes and are usually are unlikely to cause infection. Unlike virulent strains of the top seven STEC serogroups, avirulent strains are more prevalent in the raw meat samples. The presence of these avirulent strains in the raw meat samples constitutes a significant challenge for the testing laboratories in the regulatory agency and beef industry. The presence of avirulent strains in food samples reduces the assay accuracy, resulting in product hold-up for further confirmation by a culture-based method. The Microbiology Laboratory Guidebook (MLG-5C) is the standard method for detecting the top seven STEC serogroups. Based on USDA, FSIS cost-benefit analysis, the currently available STEC detection method has a false positive rate of 81-100%, which results in a loss of around $47 million in raw beef products.

Infection by STEC strains results in bloody diarrhea, hemolytic uremic syndrome and renal failure. According to the CDC, national enteric disease surveillance report E. coli O26 and O111 are responsible for 16% and 10.7% of STEC cases, respectively. According to the CDC, national enteric disease surveillance report E. coli O121 are responsible for 4.7% of total STEC cases. Previously, single nucleotide polymorphisms (SNPs) in the 0-antigen gene has been identified and associated with virulent properties of STEC strains. What is needed in the art is an assay for the differentiation of virulent strains of E. coli O26, E. coli O121, E. coli O103, and E. coli O111 from avirulent O26, O111, O103 and O121 strains.

### SUMMARY

Disclosed herein is a method for detecting virulent Shiga toxin-producing E. coli (STEC) O26 strain in a biological sample comprising the steps of: (i) enriching bacterial concentration of the biological sample to result in an enriched sample; (ii) isolating DNA from said enriched biological sample; and (iii) detecting virulent O26 in said isolated DNA sample using real-time PCR high-resolution melt curve assay or probe-based assays; wherein said real-time PCR comprises at least one primer pair, wherein said primer pair comprises SEQ ID NO: 1 and SEQ ID NO: 2.

Also disclosed herein is a method for detecting virulent Shiga toxin-producing E. coli (STEC) O111 strain in a biological sample comprising the steps of: (i) enriching bacterial concentration of the biological sample to result in an enriched sample; (ii) isolating DNA from said enriched biological sample; and (iii) detecting virulent O111 in said isolated DNA sample; wherein detection occurs via real-time PCR high-resolution probe-based assay and further wherein assay comprises a probe, wherein said probe comprises SEQ ID NO: 13 or 14, or both.

Further disclosed is a method for detecting virulent Shiga toxin-producing E. coli (STEC) O103 strain in a biological sample comprising the steps of: (i) enriching bacterial concentration of the biological sample to result in an enriched sample; (ii) isolating DNA from said enriched biological sample; and (iii) detecting virulent O103 in said isolated DNA sample; wherein detection occurs via real-time PCR probe-based assays; wherein said real-time PCR comprises at least one primer pair; wherein said primer pair comprises SEQ ID NO: 7 and SEQ ID NO: 8; or SEQ ID NO: 22 and SEQ ID NO: 23; or SEQ ID NO: 25 and 26.

Disclosed herein is a method for detecting virulent Shiga toxin-producing E. coli (STEC) O121 strain in a biological sample comprising the steps of: (i) enriching bacterial concentration of the biological sample to result in an enriched sample; (ii) isolating DNA from said enriched biological sample; and (iii) detecting virulent O121 in said isolated DNA sample; wherein detection occurs via real-time PCR probe-based assays; wherein said real-time PCR comprises at least one primer pair; wherein said primer pair is SEQ ID NO: 9 or SEQ ID NO: 10.

Disclosed herein is a kit comprising a primer pair, wherein the primer pair comprises SEQ ID NO: 3 and SEQ ID NO: 4 and wherein the kit further comprises SEQ ID NO: 13 and 14.

Also disclosed is a kit comprising a primer pair, wherein the primer pair comprises SEQ ID NO: 7 and SEQ ID NO: 8.

Further disclosed is a kit comprising a primer pair, wherein the primer pair comprises SEQ ID NO: 9 and SEQ ID NO: 10.Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-D shows a high-resolution melt assay for specific identification of E. coli O26 isolates and their differentiation of virulent and avirulent strains: 1a. Melting peaks; lb. Normalized melting peaks; 1c: Melting curves; ld: Normalized melting curves; le: Difference plot.

FIG. 2A-D shows a high-resolution melt assay for specific identification of E. coli O111 isolates and their differentiation of virulent and avirulent strains: 2a. Melting peaks; 2b. Normalized melting peaks; 2c. Melting curves; 2d. Normalized melting curves.

FIG. 3 shows forward and reverse primers for O26 (SEQ ID NOS: 1 and 2) as well as where the primer hybridization sites on nucleotides 1-360 of SEQ ID NO: 5 occur, which is the antigen gene cluster for E. coli strain O26 fnl1 gene.

FIG. 4 shows forward and reverse primers for O111 (SEQ ID NOS: 3 and 4) as well as where the primer hybridization sites on nucleotides 1-780 of SEQ ID NO: 6 occur, which is the antigen gene cluster for E. coli strain O111 wbdK gene.

FIG. 5A-C shows normalized melting information for O103 HRM assay. 5A shows normalized melting peaks for O103 HRM assay using pure culture strains. FIG. 5B shows normalized melting curves for O103 HRM assay using pure culture strains. FIG. 5C shows normalized melting curves of O103 HRM assay using enriched food samples.

FIG. 6A-C shows normalized melting information for O121 HRM. FIG. 6A shows normalized melting peaks for O121 HRM assay using pure culture strains. FIG. 6B shows normalized melting curves for O121 HRM assay using pure culture strains. FIG. 6C shows normalized O121 melting curves of O121 HRM assay using enriched food samples.

FIG. 7 shows enriched beef samples when tested using LightCycler® 480 High-Resolution Melting Master.

FIG. 8 shows enriched beef samples when tested using Apex qPCR 2x GREEN master mix supplemented with 0.25 μL of 1×EvaGreen.

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### DEFINITIONS

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “STEC” or “Shiga toxin-producing Escherichia coli” refers to a group of E. coli strains with the ability to produce Shiga toxin via the expression of stx1 and stx2 genes. STECs are broadly divided into E. coli serotype O157 and non-O157 serogroups. Specifically discussed herein are the O26, O121, O103, and O111 strains.

As used herein, “multiplex” refers to refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

As used herein, “enrichment” refers to conditions favoring the growth of a particular microorganism. For example, in one embodiment, a method of the present invention may benefit from an enrichment step whereby bacterial cells or a solution obtained by homogenizing a biological sample and containing one or more target bacterial cells or species are placed in an enrichment medium to allow for the growth of the target bacterial species or strains for the purposes of detection of the bacterial cells or species.

As used herein, the term “subject,” “patient,” or “organism” includes humans and mammals (e.g., mice, rats, pigs, cats, dogs, and horses). Typical subjects for which methods of the present invention may be applied will be mammals, such as humans. A wide variety of subjects will be suitable for veterinary, diagnostic, research, or food safety applications, e.g., humans; livestock such as cattle, sheep, goats, cows, swine, and the like; poultry such as chickens, ducks, geese, turkeys, and the like; and domesticated animals, particularly pets such as dogs and cats. The term “living subject” refers to a subject as noted above or another organism that is alive.

As used herein, the term “culture media” or “media” refers to liquid, semi-solid, or solid media used to support bacterial cell growth in a non-native environment. Further, by culture media is meant a sterile solution that is capable of sustaining and/or promoting the division or survival of such cells. Suitable culture media are known to one of skill in the art, as discussed herein. The media components may be obtained from suppliers other than those identified herein and may be optimized for use by those of skill in the art according to their requirements. Culture media components are well known to one of skill in the art and concentrations and/or components may be altered as desired or needed.

In certain embodiments, sequences of the present invention, including primer sequences, target sequences and Internal amplification control (IAC) sequences may be identical to the sequences provided here in or comprise less than 100% sequence identity to the sequences provided herein. For instance, primer sequences, target sequences or IAC sequences of the present invention may comprise 90% identity to the sequences provided herein.

The terms “identical” or “percent identity,” in the context of two or more nucleic acids or sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/ or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed. The sequence can comprise or consist of those sequences disclosed herein.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated region that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The term “recombinant” when used with reference, e.g., to a cell, or nucleic acid indicates that the cell or nucleic acid has been modified by the introduction, by natural or artificial means, of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In some embodiments, recombinant sequences may also include nucleic acids, proteins, or recombinant genomes, such as bacterial genomes. In certain embodiments, a “recombinant” bacterium or cell may refer to a bacterial cell into which a stx gene or nucleic acid has been inserted, for example by a lambdoid phage, conferring the ability of the bacterial cell to produce shiga toxin.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### Methods and Compositions for Detection of Virulent Strains of E. Coli

The present invention provides methods for detecting and differentiating between virulent and non-virulent strains of Shiga-toxin producing Escherichia coli (STEC) O26, O121, O103, and O111 antigens in a biological sample. Disclosed herein are primers specific for these strains, as well as methods of detecting STEC strains O26, O121, O103, and O111 strains.

STECs are zoonotic pathogens found in the intestinal tract and feces of beef cattle and ruminants, and thus can be introduced into food products of animal origin during slaughter. Beef products contaminated with animal feces have been associated with STEC infections in human. These pathogens have also been reported to contaminate milk, cheese, and other dairy products. STEC infections lead to a variety of illnesses with varying severity, including diarrhea, hemorrhagic colitis (bloody diarrhea), hemolytic uremic syndrome (kidney failure), and death resulting in STEC strains causing human illness to be included as notifiable pathogens to the Nationally Notifiable Diseases Surveillance System in 2000.

STECs are broadly divided into E. coli serotype O157 and non-O157 serogroups. In the last decade, the non-O157 serogroup has emerged as a major food-borne pathogen of concern worldwide, responsible for 63%, 74%, 82%, and 80% of the total STEC infections in Canada, Denmark, Germany, and the Netherlands, respectively. To date, a large number of STEC serogroups have been identified, but not all are pathogenic to humans. The frequency of infections of STEC serogroups is variable, with six non-O157 STEC serotypes being most commonly reported: O26 (26%), O103 (22%), O111 (19%), O121 (6%), O45 (5%), and O145 (4%), leading to their classification by the USDA as adulterants (zero tolerance) in non-intact raw beef products.

STECs produce Shiga toxins via the expression of stx1 and stx2 genes, which are transferred to a bacterial cell by lambdoid phages that integrate into the bacterial chromosome. A total of 10 known stx subtypes are presently known. The stx1 and stx2 are further divided into multiple subtypes, in which stx1 has three subtypes (stx1a, stx1c, and stx1d), and stx2 has seven subtypes (stx2a, stx2b, stx2c, stx2d, stx2e, stx2f, and stx2 g). Although commercial kits are presently available for identification of Shiga toxin genes, the high genetic diversity of the stx subtypes results in subtypes that may not necessarily be detected by the various assays (Feng et al., Appl Environ Microbiol 77:6699-6702, 2011; Margot et al., J Food Protection 76:871-873, 2013).

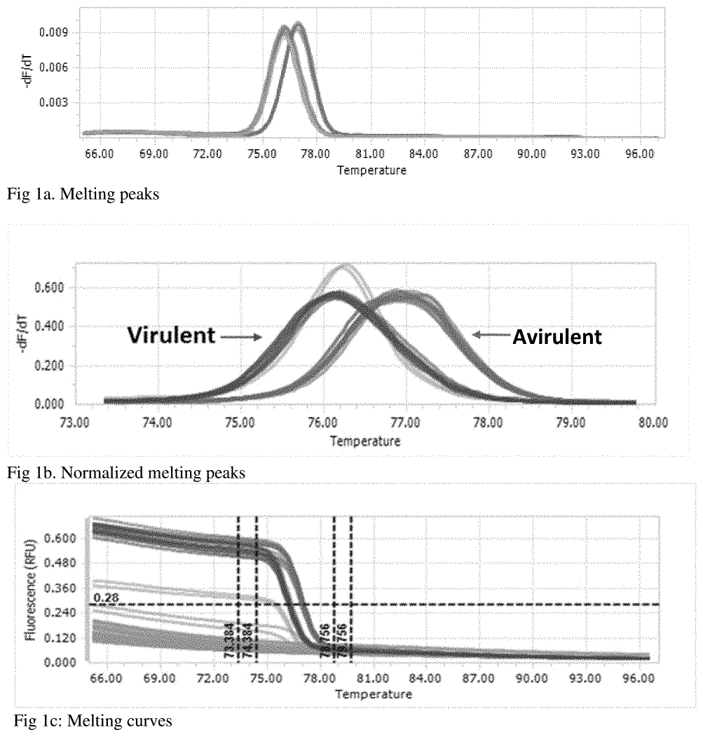

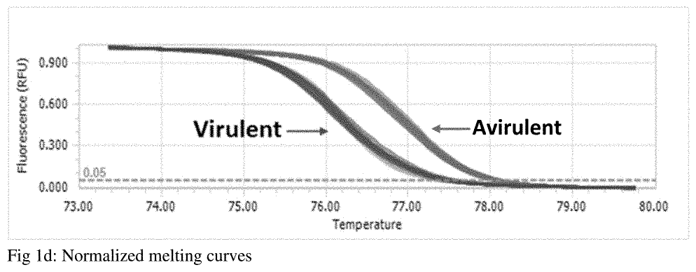

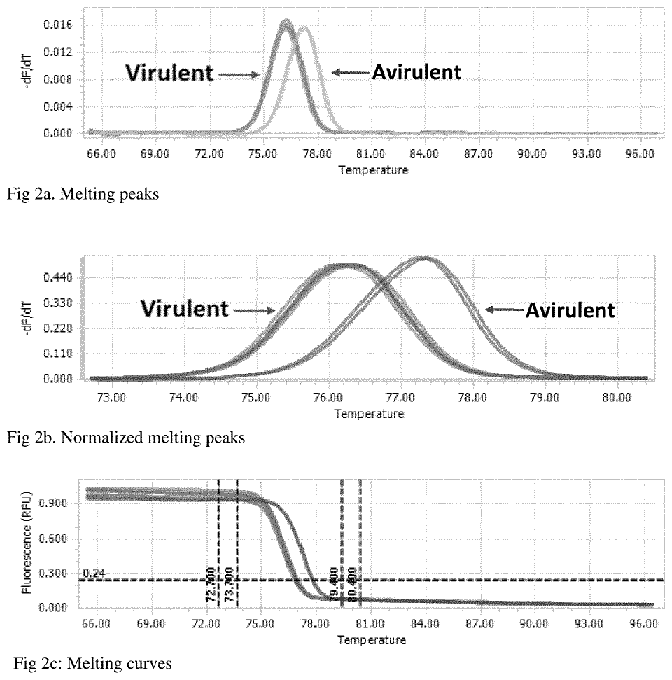

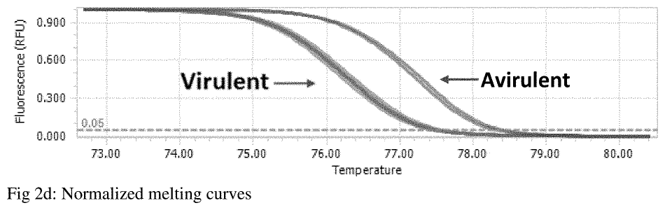

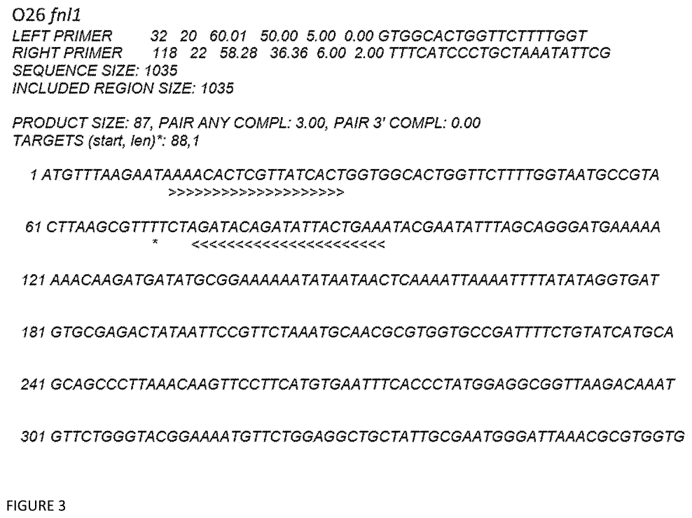

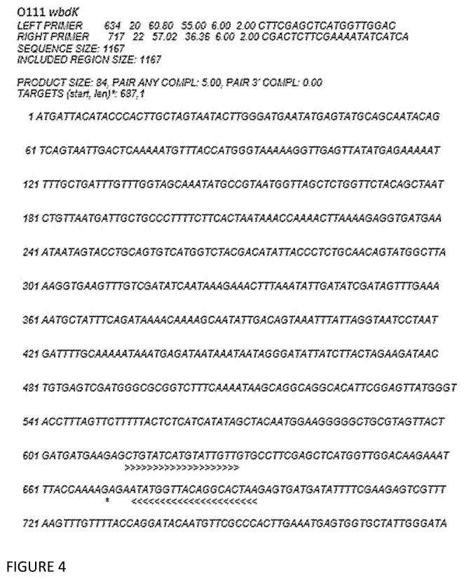

In accordance with the invention, both virulent and avirulent strains of O26, O121, O103, and O111 can be specifically and reliably detected in a biological sample. Using the methods described herein, these STECs may be specifically and reliably detected in a biological sample at low concentrations and in minimal time, thus enabling rapid and low-cost simultaneous detection of multiple pathogenic bacteria. Importantly, melting curves can further distinguish between virulent and avirulent strains. For example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. or more difference in the melting peaks can be used to can be used to separate virulent from avirulent strains of O26, O121, O103, and O111. This can be seen in FIGS. 1 and 2.

The methods described herein may be used to test a multitude of biological samples, for example food products. In one embodiment, a biological sample may be meat such as beef, beef stew meat, beef trimmings, chicken, turkey, or the like. A biological sample may also include produce such as various vegetables and fruits, such as alfalfa sprouts, spinach, lettuce, or juices from vegetable or fruits such as apple cider. As used herein, a “biological sample” or “sample” may also include clinical samples such as blood and blood parts including, but not limited to serum, plasma, platelets, or red blood cells; sputum, mucosa, tissue, cultured cells, including primary cultures, explants, and transformed cells; biological fluids, stool, and urine. A biological sample may also include sections of tissues such as biopsy and autopsy samples, and frozen sections taken for histologic purposes. A biological sample may be obtained from a eukaryotic organism, for example a mammal, including humans, cows, pigs, chickens, turkeys, ducks, geese, dogs, goats, and the like. Any tissue appropriate for use in accordance with the invention may be used, for instance, skin, brain, spinal cord, adrenals, pectoral muscle, lung, heart, liver, crop, duodenum, small intestine, large intestine, kidney, spleen, pancreas, adrenal gland, bone marrow, lumbosacral spinal cord, or blood.

In some embodiments, methods of the present invention may comprise the steps of: i) enriching a bacterial concentration in a test sample by incubating the sample aerobically at approximately 42° C., for instance 37° C., 38° C., 39° C., 40° C., 41° C., 42° C., 43° C., 44° C., or 45° C. in an enrichment media such as described herein; ii) isolating DNA from the enriched sample; and iii) detecting sample DNA using the specific primer sets as described herein.

During the sample enrichment step, a biological sample such as a food sample or other clinical sample, may be collected and diluted in a buffer or media such as water, saline, brain heart infusion broth (BHI), tryptic soy broth (TSB), modified tryptic soy broth (mTSB) or sterile Buffered Peptone Water (BPW), among others. Media useful for culture or enrichment of STECs, Salmonella, or other food pathogens in food samples would be known by one of skill in the art. Exemplary media in accordance with the invention may include, but are not limited to, BHI, TSB, mTSB and buffered peptone water (BPW) broth. In some embodiments, a sample as described herein may be diluted at any stage in a desired buffer or solution, for example 1:10, 1:9, 1:8, 1:7, 1:6, 1:5, 1:4, 1:3, 1:2, or 1:1.

Antibiotics may be used in the enrichment of STECs in order to provide a selective advantage for pathogens to grow among any other bacteria present in a food or environmental sample. The use of antibiotics in enrichment broth may hinder the growth of other existing bacteria and simultaneously promote the selective growth of pathogens to be detected in the assay of the present invention. Selection of suitable antibiotics may ensure that the growth of the selected pathogen is not inhibited, thereby ensuring that the sample enrichment time is not lengthened unnecessarily. In some embodiments, antibiotics may be added to a sample or medium such as an enrichment medium or a culture medium. For example, VCC supplement, containing vancomycin, which deters the growth of Gram-positive bacteria, cefixime, which suppresses the growth of Proteus spp., and/or cefsulodin, which inhibits Pseudomonas spp.; novobiocin, acriflavine, penicillin, streptomycin, chloramphenicol, gentamycin, and the like. Antimycotic compounds may include, but are not limited to Fungizone or other suitable compound. Any of these compounds may be used alone or in combination where appropriate. Any suitable concentration of antibiotic or antimycotic may be used, for example 10 mg/L, 9 mg/L, 8 mg/L, 7 mg/L, etc. In other embodiments, a diluted sample may be homogenized using an appropriate device, such as a Stomacher, for a short time period (such as 2 minutes) in order to release attached cells. The homogenized samples may be incubated aerobically, such as at 42° C. or other temperature suitable for a bacterial strain, for anywhere from 4 to 12 hours to allow for enrichment (recovery and growth of target bacterial species). For example, samples may be incubated for 4 hours, 5 hours, 6 hours, 7 hours, 8 hours, 9 hours, 10 hours, 11 hours, or 12 hours, or 15 hours the like.

During the DNA isolation step as described herein, DNA from an enriched sample may be isolated using any method available as would be known by one of skill in the art. In one embodiment, a commercially available kit, such as PrepMan® Ultra Sample Preparation Reagent (Applied Biosystems, Life Technologies), or DNeasy® Power Food Microbial Kit (Qiagen, Hilden, Germany) may be used to isolate DNA. According to one embodiment, suspended food particles may be separated from the media, for instance through filtration or centrifugation of the enriched sample, for example at 100×g. The obtained supernatant may then be used for DNA isolation as described herein.

### Isolation and Amplification of DNA

Methods such as polymerase chain reaction (PCR and RT-PCR) and ligase chain reaction (LCR) may be used to amplify nucleic acid sequences directly from genomic material, such as genomic DNA, mRNA, cDNA, or from genomic libraries, or cDNA libraries.

In some embodiments, the primers are not labeled, and the amplicons may be visualized, detected, and/or analyzed by real-time PCR using a intercalating dye based approach following their melting temperature, for example by generation of melt curve assays or plots or a dual-labeled probe based approach (i.e., TaqMan, Molecular beacon, Sunrise probe). In other embodiments, an amplicon may be visualized according to size, e.g., using agarose gel electrophoresis. In some embodiments, ethidium bromide staining of the PCR amplicons following size resolution allows visualization of the different size amplicons. Such an approach may be referred to as end-point PCR. Conventional end-point PCR, while suitable for amplification and detection of a target DNA or sequence, may require extensive sample enrichment time due to the higher copy number of target DNA molecules needed for detection. This translates to a higher number of target cells, which, in turn, translates to longer enrichment times. In some embodiments, the primers of the invention may be radiolabeled, or labeled by any suitable means (e.g., using a non-radioactive fluorescent tag), to allow for rapid visualization of amplicons of different sizes following an amplification reaction without any additional labeling step or visualization step.

In accordance with the invention, a PCR assay as described herein may be multiplexed in order to combine multiple reactions into a single assay. For example, a multiplex assay may enable amplification of multiple target sequences using a number of PCR primer pairs, such as one or more primers set forth in the Examples. One of skill in the art will understand that the reaction conditions for each individual reaction in a multiplex assay will necessarily be similar in order to achieve efficient amplification of each target. Optimization or other testing of each individual primer pair may be necessary. For the development of a multiplex PCR assay such as described herein, a large number of primer-pairs has to be tested for each target in order to determine the optimum primer that will produce the best result. Out of multiple PCR primers that work for a particular multiplex assay, a final set of primer pairs for a multiplex assay may be selected based on specific criteria, including, but not limited to, (1) maximum melting temperature difference (at least 2-3° C.) from neighboring peaks; (2) higher PCR amplification efficiency; (3) amplicon size; and (4) size of melt peak formed on the melt curve plot.

In some embodiments, a bacterial species such as a STEC species or serogroups as described herein may be detected based on the level of a particular RNA or DNA in a biological sample. Any of the primers described herein and set forth as SEQ ID NOs:1-4 or 7-10 may be used for detection, diagnosis, and determination of the presence of such a bacterial species. An amplified nucleotide may then be detected and distinguished for other sequences using techniques described herein.

A PCR assay may include a number of reagents and components, including a master mix and nucleic acid dye or intercalating agent. In some embodiments, an exemplary PCR master mix may contain template genomic material, such as DNA, PCR primers, salts such as MgCl2, a polymerase enzyme, and deoxyribonucleotides. One of skill in the art will be able to identify useful components of a master mix in accordance with the present invention. In one embodiment, a master mix such as MeltDoctor™ HRM Master Mix (Applied Biosystems, Life Technologies), which contains the high-resolution melting (HRM) dye, SYTO®9 may be used.

During real-time PCR detection, PCR may be performed in any reaction volume, such as 10 μL, 20 μL, 30 μL, 50 μL, 100 μL, or the like. Reactions may be performed singly, in duplicate, or in triplicate. PCR thermal cycling conditions are well known in the art and vary based on a number of factors. As described herein, an exemplary two-step amplification protocol may include, for example, an initial denaturation at 94° C. for 10 min; 40 cycles of 94° C. for 30 s, 60° C. for 45 s; and a melt curve step performed at the end of the PCR (from 60° C. to 95° C., with gradual temperature increments of 0.04-0.1° C/s). Melt curve plots may be prepared by plotting the negative derivative of fluorescence (−Rn) versus temperature. Mean Tm values for each product may be calculated by averaging the Tm values from duplicate runs of two replications. Any thermal cycling program may be designed as appropriate for use with the particular primers for detection of particular bacterial species as would be understood by one of skill in the art.

Test samples or assays as described herein may be compared to a control or reference sample, such as a positive control, in order to accurately determine the presence and/or amount of a particular pathogen such as a STEC or pathogenic or antibiotic resistant bacterial species. In addition, a reaction control may be used, such as an IAC, in order to avoid false negative results and thereby increase the reliability of an assay. Use of an IAC in a reaction provides assurance that a negative result for a target is truly a negative result rather than due to a problem or break-down in the reaction. Because the signal for the IAC should always be generated, even when the target signal is not generated (i.e., the target organism or DNA is not present in the sample), this would indicate that a negative target signal is indeed a negative result. An IAC may be useful in diagnostic assays because food matrices may harbor inhibitory components that may interfere with PCR amplification, leading to false negative results.

Short oligonucleotides such as an IAC molecule as described herein may be amplified at a much higher amplification efficiency (>100%) and thus may be preferentially amplified in a multiplex PCR reaction. To overcome this issue, an IAC molecule may be added to a multiplex reaction at the lowest possible concentration (10-20 fg), facilitating preferential amplification of the desired target DNA.

### Modification of Nucleic Acids

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules are capable of forming an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are known in the art and described by Sambrook, et al. (1989), and by Haymes et al. (1985).

Departures from complete complementarity are permissible, as long as the capacity of the molecules to form a double-stranded structure remains. Thus, in order for a nucleic acid molecule or a fragment of the nucleic acid molecule to serve as a primer or probe, such a molecule or fragment need only be sufficiently complementary in sequence to be able to form a stable double-stranded structure under the particular solvent and salt concentrations employed.

Appropriate stringency conditions that promote DNA hybridization are well known to one of skill in the art and may include, for example, 6x sodium chloride/sodium citrate (SSC) at about 45° C., followed by a wash of 2×SSC at 50° C. The salt concentration in the wash step may be selected from a low stringency of approximately 2×SSC at 50° C. to a high stringency of about 0.2×SSC at 50° C. The temperature in the wash step may be increased from low stringency conditions at room temperature, about 22° C., to high stringency conditions at about 65° C. The temperature and/or salt conditions may be varied as appropriate for optimum results. In accordance with the invention, a nucleic acid may exhibit at least from about 80% to about 100% sequence identity with one or more nucleic acid molecules as described herein, for example at least from about 80%, about 85%, about 90%, about 95%, about 98%, about 99%, or about 100% sequence identity. One of skill in the art will understand that stringency may be altered as appropriate to ensure optimum results.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

SEQ ID NOS: 1-2, 3-4, 7-8, 25-26, and 27-28 set forth particular primer pairs Specifically disclosed are variants of these primers which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Stated another way, disclosed are primers which differ by 1, 2, 3, 4, 5, 6, 7, 8, or 10 nucleotides from SEQ ID NOS: 1-2, 3-4, 7-8, 25-26, and 27-28. Those of skill in the art readily understand how to determine variations that can occur in the primers which still allow for the retention of their functionality.

### Detecting the Presence of a Bacterial Species in a Biological Sample

In some embodiments, the oligonucleotides of the invention may be detectably labeled, thereby also functioning as probes. Detectable labels may include, but are not limited to, radiolabels, fluorochromes, including fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxy fluorescein (6-FAM) or N,N,N′,N′-tetramethyl-6-carboxyrho-damine (TAMRA); radioactive labels such as 32P, 35S, and 3H), and the like. In some embodiments, a detectable label may involve multiple steps (e.g., biotin-avidin, hapten-anti-hapten antibody, and the like). A primer useful in accordance with the invention may be identical to a particular bacterial target nucleic acid sequence and different from other bacterial sequences. In another embodiment, a primer and/or probe useful in accordance with the invention may enable distinction between a nucleic acid sequence from a virulent and an avirulent STEC strain, such as in O26, 0111, O103, and O121.

### Diagnostic Tests and Kits

Disclosed are kits that may comprise one or more of the primer pairs disclosed herein. For example, the kit may comprise primers comprising SEQ ID NOS: 1 and 2, or variants thereof. Another kit may comprise primers comprising SEQ ID NOS: 3 and 4, or variants thereof. Another kit may comprise primers comprising SEQ ID NOS: 7 and 8, or variants thereof. Another kit may comprise primers comprising SEQ ID NOS: 9 and 10, or variants thereof. Another kit may comprise primers comprising SEQ ID NOS: 22 and 23, or variants thereof. Another kit may comprise primers comprising SEQ ID NOS: 25 and 26, or variants thereof. Yet another kit may comprise any combination of these primer pairs, such as SEQ ID NOS: 1-4; SEQ ID NOS: 1-2 and 7-8; SEQ ID NOS: 1-2 and 9-10; SEQ ID NOS: 1-2 and 22-23; SEQ ID NOS: 1-2 and 25-26; SEQ ID NOS: 3-4 and 7-8; SEQ ID NOS: 3-4 and 9-10; SEQ ID NOS: 3-4 and 22-23; SEQ ID NOS: 3-4 and 25-26; SEQ ID NOS: 1-4 and 7-8; SEQ ID NOS: 1-4 and 9-10; SEQ ID NOS: 1-4 and 22-23; SEQ ID NOS: 1-4 and 25-26; SEQ ID NOS: 3-4 and 7-10; SEQ ID NOS: 3-4 and 22-23; SEQ ID NOS: 3-4 and SEQ ID NOS: 25-26; or all of SEQ ID NOS: 1-4, 7-10, 22-23, and 25-26, or combinations or variants thereof. Additional primer sets not included in this list may also be included.

The invention further provides diagnostic reagents and kits comprising one or more such reagents or components for use in a variety of diagnostic assays, including for example, nucleic acid assays, e.g., PCR or RT-PCR assays. Such kits may preferably include at least a first primer pair as described herein, and means for detecting or visualizing amplification of a target sequence. In some embodiments, such a kit may contain multiple primer pairs as described herein for the purpose of performing multiplex PCR or RT-PCR for detection of multiple target sequences. Primer pairs may be provided in lyophilized, desiccated, or dried form, or may be provided in an aqueous solution or other liquid media appropriate for use in accordance with the invention.

Kits may also include additional reagents, e.g., PCR components, such as salts including MgC12, a polymerase enzyme, and deoxyribonucleotides, and the like, reagents for DNA isolation, or enrichment of a biological sample, including for example media such as water, saline, BHI, TSB, BPW, or the like, as described herein. Such reagents or components are well known in the art. Where appropriate, reagents included with such a kit may be provided either in the same container or media as the primer pair or multiple primer pairs, or may alternatively be placed in a second or additional distinct container into which the additional composition or reagents may be placed and suitably aliquoted. Alternatively, reagents may be provided in a single container means.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### EXAMPLE 1: High-Resolution Melt Assay for Detection of Virulent E. coli O26 and O111 Strains

Serogroup specific O26 and O111 primers showed 100% specificity. Virulent strains of E. coli O26 and O111 formed distinct melt profiles in the normalized and differential melting curves plots. Melt profiles of virulent strains clustered separately and were easily distinguishable from those of avirulent O26 and O111 strains. The O26 assays showed 75% specificity and 100 sensitivity, whereas the O111 assay showed 100% specificity and 100% sensitivity for the identification of virulent strains. The assay developed for specific identification O26 and O111 serogroups and differentiation of positive isolates into virulent and avirulent strains can be used to improve food safety and reduce response time during foodborne outbreaks.

### Materials and Methods

Bacterial strains: Virulent and avirulent STEC strains were received from Roman L. Hruska U.S. Meat Animal Research Center (Clay Center, NE, USA). Other bacterial strains were obtained from Florida State University, Food Microbiology Lab Culture collection. Bacterial strains were sub-cultured at 37° C. overnight in Tryptic Soy broth (TSB) (Hardy Diagnostics, Santa Maria, Calif., USA).

Bacterial isolates used (see Table 3): Acinetobacter pittii 27B MM, Acinetobacter schindleri 27B MC1, Acinetobacter schindleri 27B MC2, Aeromonas hydrophila 20A MM2, Alcaligenes faecalis 22B MC2 1, Bifidobacterium bifidum 11863, Citrobacter freundii 14B MC1, Citrobacter freundii 16B MM1, E. coli 25922, E. coli O103-1 hSTEC 05, E. coli O103-2 MDR 0089 (USMARC_GB_STEC O45), E. coli O103-3 Mar 125B (USMARC_GB_STEC 046), E. coli O103 302.1, E. coli O103 33, E. coli O103 612.1, E. coli O103 621.2, E. coli O103 745.1, E. coli O103 75.2, E. coli O103 802.1, E. coli O111 C4-462-2_7095, E. coli O111 F-A 790.1, E. coli O111:H8-hSTEC 08, E. coli O111 739.3, E. coli O121 2′ nph1_12738, E. coli O121 211-1, E. coli O121 219.5, E. coli O121 256-1, E. coli O121 3′ C4-63-1_3218, E. coli O121 4 V2-G2 1-C 16.3, E. coli O121 508.3, E. coli O121 75.3, E. coli O121 785.2, E. coli O121 967.1, E. coli O145 1 hSTEC 22, E. coli O145 170-2, E. coli O145 2 May 109, E. coli O145 219.7, E. coli O145 3 C4-69-1 3275, E. coli O145 317.2, E. coli O145 56.2, E. coli O145 690.1, E. coli O157 221.2, E. coli O157 645.V1, E. coli O157 766. V1, E. coli O157 E0018, E. coli O26 16.2, E. coli O26 699.1, E. coli O26 859.V3, E. coli O26 946.1, E. coli O26 97.1, E. coli O26 hSTEC-03, E. coli O26 Imp 133.1, E. coli O26 May 063

(USMARC_GB_STEC 021), E. coli O45 151.1, E. coli O45 219.1, E. coli O45 235.1, E. coli O45 623.3, E. coli O45 978.1, E. coli 14B MM, E. coli 27A MNC, Enterobacter hormaechei 25B MC1, Enterobacter hormaechei 25B MC2, Enterobacter lignolyticus 15B MC2 -1, Enterobacter lignolyticus 15B MM1, Enterococcus faecalis 15A MNC1, Enterococcus faecalis 15B MC2 2, Klebsiella pneumoniae 24A MNC2, Lactobacillus plantarum, Lelliottia amnigena 15B MC1 1, Morganella morganii 15A MNC2, Morganella morganii 16B MC1 1, Obesumbacterium proteus 17B MM2, Obesumbacterium proteus 28A MNC, Pantoea alhagi 25A MNC, Proteus mirabilis 16B MC1-2, Proteus mirabilis 16B MC2 1, Providencia alcalifaciens 17B MC2-2, Salmonella Typhimurium 14028, Serratia marcescens 28B MC2, Serratia marcescens 28B MM, Stenotrophomonas maltophilia 29B MC1, Stenotrophomonas maltophilia 29B MC2, Vibrio alginolyticus 18B MC2-1, Vibrio alginolyticus 18B MM, Vibrio fluvialis 24B MM, Vibrio parahaemolyticus 24B MC1 and Vibrio parahaemolyticus 24B MC2

DNA extraction: Genomic DNA from overnight cultures were extracted using Extracta DNA Prep for PCR (QantaBio, Beverly, MA, USA). Bacterial cell pellet from one milliliter of bacterial cultures were obtained by centrifugation at 13,000 ×g for 1 min. Obtained cell pellets were lysed with 50 μl of extraction reagent and heating at 95° C. for 10 minutes on dry bath. Samples were cooled at room temperate for 5 minutes and 50 μl of stabilization buffer was added and samples were centrifuged at 13,000 ×g for 2 min. Fifty microliters of supernatant containing bacterial DNA was transferred to a new Eppendorf tube. The purity and concentration of isolated DNA were measured by a Nanodrop Lite Spectrophotometer (Thermo Fisher, Wilmington, DE, USA). DNA samples were diluted to 10 ng/μl concentration and used for real-time PCR.

Primer design: The real-time PCR primer for this study was designed using the Primer3 software (Untergasser et al., 2012). Primer-pairs were designed flanking the SNP identified in previous publication (Norman et al., 2012). The specificity and cross-amplification potential of the designed primer-pairs was tested using NCBI/Primer-BLAST tool. All oligonucleotides (Table 1) used in this study were commercially synthesized by IDT (Coraville, Iowa, USA).

TABLE 1
Primers Used

| | | Product |
|---|---|---|
| Name | Primer sequence | Size |
| O26fnl1- | 5′-GTG GCA CTG GTT | 87 |
| 32F | CTT TTG GT-3′ | |
| | (SEQ ID NO: 1) | |
| O26-219R | 5′-ACCACGCGTTGCA | |
| | TTTAGAA3′ | |
| | (SEQ ID NO: 2) | |
| O111 wbdK- | 5′-CTT CGA GCT CAT | |
| 634F | GGT TGG AC-3′ | |
| | (SEQ ID NO: 3) | |
| O111 wbdK- | 5′-CGA CTC TTC | |
| 717R | GAA AAT ATC A-3′ | |
| | (SEQ ID NO: 4) | |

Real-time PCR: Real-time high-resolution melt assays were performed on LightCycler® 96 real-time PCR instrument (Roche Diagnostics Corp., Indianapolis, USA) with 2x LightCycler® 480 High Resolution Melting Master (Roche Diagnostics Corp., Indianapolis, USA). Additionally, a 10 μl real-time PCR reaction mixture consisted of 20 ng of DNA, 0.5 μM forwards and reverse primers and 2.5 mM MgCl2. A two-step PCR protocol was used for the amplification of target sequence. The amplification protocol consisted of an initial denaturation step at 94 ° C. for 600 seconds, 40 cycles of 95 ° C. for 15 s and 62 ° C. for 30 s. A HRM was performed at the end of the PCR amplification starting from 60 ° C. to 95 ° C., with gradual temperature increments of 0.04 ° C/s (25 reading/° C.). Signal from PCR amplification and HRM were collected in the ResoLight channel of the real-time instrument. HRM analysis for E. coli O26 was performed with a pre-melt region 73.3 — 74.3 ° C. and a post-melt region 78.7-79.7 ° C. Whereas HRM analysis for E. coli O111 was performed with a pre-melt region 71.5-72.5 ° C. and a post-melt region 78.2 — 79.2 ° C.

Inoculum preparation and enumeration: Six strains of O26 and four strains of O111 were chosen for inoculating the food samples (Table 2). Each strain was individually cultured at 37 ° C. in 10 mL TSB (Hardy Diagnostics, Santa Maria, Calif., USA). At end of the incubation period cultures were serially diluted and enumerated on tryptic soya agar (Hardy Diagnostics, Santa Maria, Calif., USA). Broth cultures after enumeration were stored in refrigerator while awaiting the colony counts and TSA plates were aerobically incubated at 37° C. Counts from TSA plates were used to calculate appropriate dilution and volume needed for spiking food samples at 10CFU. Further, the volume used to spike food sample were spread plated on TSA agar for accurately enumerating the E. coli cells used to spike food samples.

TABLE 2
Bacterial strains used for spiking food samples.

| Stab ID | Source | EHEC Pattern |
|---|---|---|
| O26:H11 hSTEC-03 | human | 1 e h |
| O26:H11 Imp 133.1 | beef | 1 e h |
| O26:H11 May 063 | beef | 1 e h |
| (USMARC_GB_STEC 021) | | |
| O26 16.2 | | |
| O26 97.1 | | espK1 nleB eae nleF |
| O26 699.1 | | nleB eae nleF |
| O111:H8 hSTEC 08 | human | 1 2 e h |
| O111 C4-462-2_7095 | beef | stx eae |
| O111 F-A 790.1 | beef | 1 e h |

Food sample preparation: Ground beef (12% fat: 88% lean), beef pot roasts, and spinach were purchased from the Costco (Tallahassee, Florida). Beef pot roast were trimmed into thin strips as recommended by USDA N60 sampling protocol. Three hundred and twenty-five grams of beef sample and 25 grams of spinach samples were aseptically transferred in sterile WhirlPak filter stomacher bags (Nasco, Fort Atkinson, WI). Food samples in stomacher bags were individually spiked with 10 CFU/bag using strains of E. coli O26 and O111. Spiked food samples were allowed to attach to the food matrix by incubating samples for 15 minutes at room temperature. After attached step spiked food samples were stored at 4° C. for 24h. At the end of 24h storage samples beef samples were diluted with 975 mL whereas spinach samples were diluted with 225 mL of TSB with casamino acid and 2mg/L novobiocin at 37° C. Beef samples were hand messaged and spinach samples were stomached at 230 rpm for 2 min. Food samples after addition of enrichment broth were incubated at 37° C. and samples were collected after 15 h and 21 h enrichment time. DNA from 1.8 mL of enrichment broth were isolated using DNeasy® Power Food Microbial Kit (Qiagen, Hilden, Germany), following manufacturer instructions.

### Results and Discussion

Combination of culture, immunological and molecular methods are used for rapid identification of foodborne pathogens. In this study two high resolution melt real-time PCR assays were standardized for specific identification of virulent strains of E. coli O26 and O111. The assay developed were initially standardized using pure culture DNA samples. The specificity of developed assays were tested using 87 pure culture isolates which belonged to 19 genera. The specificity of E. coli O26 primer pair designed in this correctly identified all E. coli O26 isolates.

The HRM melt analysis for O26 assay was performed using a pre-melt region of 73.3 to 74.3° C. and post-melt region of 78.7 to 79.9° C. The sensitivity of HRM analysis was adjusted to 100% discrimination based on delta Tm discrimination. The ability of assay to discriminate between virulent and avirulent strains were tested using 8 26 strains (16.2, 97.1, 699.1, 859.V3, 946.1, hSTEC-03, Imp 133.1, May 063 (USMARC_GB_STEC 021)). The E. coli O26 HRM assay correctly discriminated between all virulent and avirulent strains except E. coli O26 97.1 (FIG. 1). This strain clustered with virulent strains. The 97.1 isolate lacked the stx genes and was positive for espK1, nleB, eae and nleF genes. It appears that this isolate (97.1) was a pathogenic isolate, which lost it stx genes at some point of time. This assay can accurately identify O26 isolates and can be used for discrimination of virulent and avirulent strains.

The E. coli O111 HRM assay developed showed 100% sensitivity and specificity. The primer designed in this study showed no cross reactivity with any of the 87 isolates spread across 19 genera and accurately identified all O111 isolates. The HRM analysis was performed with sensitivity adjusted to 100% discrimination based on delta Tm discrimination. The O111 isolates tested in this study clustered into two groups (FIG. 2). Four O111 isolates were tested (C4-462-2_7095, F-A 790.1, hSTEC 08 and 739.3) in the culture collection. The assay was accurately able to differentiate between virulent and avirulent strains.

Beef products and fresh produce are frequently associated with STEC outbreaks. Low infective dose (<10 cells) of these STEC strains and their ease of transmission through contaminated food makes them foodborne pathogen of significance concern (Etcheverria & Padola, 2013). Hence, food samples in this study were spiked at ˜10 CFU/325 g for beef samples and ˜10 CFU/25 g for spinach samples. At the time of spiking food samples, calculated amount of inoculum was spread plated on plate count agar. The inoculum counts for beef and spinach samples ranged between 5 to 19 CFU.

Beef products and fresh produce have high bacterial load, varying fat content and PCR inhibitors making detection of foodborne pathogens at lower concentration a challenging task and necessitating use of enrichment step (Li et al., 2017; Singh et al., 2019; Singh & Mustapha, 2015). The aerobic plate count of spinach, beef trims and ground beef were (5.3-5.5 log CFU/g), (4.5 — 6.0 log CFU/g) and (5.6 — 6.0 log CFU/g), respectively. STEC strains (O26:H11 hSTEC-03, 026:H11 Imp 133.1, 26:H11 May 063 (USMARC_GB_STEC 021), O26 16.2, 26 97.1, 26 699.1, 0111 C4-462-2_7095 and O111 F-A 790.1) were detected following a 15 h enrichment period. During enrichment of spiked food samples, the food microbiota and spiked pathogen competes with each other for same nutrients. Therefore, food samples with higher microbial load may require longer enrichment time.

Antibiotics (e.g. novobiocin, cefoxitin, vancomycin, cefsulodin) are often added to deter growth of commensal microbiota and facilitate growth of target pathogen (Boer & Heuvelink, 2000). The STEC strains are resistant to these antibiotics, however based on experience these antibiotics can still slowdown growth of STEC strains at recommended concentrations (Singh et al., 2019; Singh & Mustapha, 2015). Therefore, novobiocin was added to enrichment broth at a reduced concentration (2 mg/L).

The PrepMan ultra sample preparation reagent is a commonly used method for isolation of bacterial DNA from enriched food samples (Fratamico et al., 2011; Wasilenko et al., 2012). This DNA isolation method is rapid and results in DNA samples which are suitable for most PCR based foodborne pathogen detection methods. However, the HRM assays are very sensitive to quality of DNA quality. Presence of impurities in DNA samples can interfere with grouping of melt profiles during the HRM analysis step. Therefore, in this study DNeasy PowerFood Microbial Kit was used for isolation of microbial DNA from enriched food samples. Depending on number of samples, DNA isolation process using the DNeasy PowerFood Microbial Kit can take from 4 to 6 h. Nevertheless, the DNeasy PowerFood Microbial Kit results in higher purity DNA which is more suited for HRM assays. Previous studies have shown superior performance of DNeasy PowerFood Microbial Kit over other DNA isolation kit (Jana et al., 2020; Liu et al., 2018; Lusk et al., 2013).

### EXAMPLE 2: Detection of virulent E. coli O103 and O121 strains

Introduction: Seven virulent strains of Shiga toxin-producing Escherichia coli (STEC) are considered as adulterant in non-intact red meat. Infection caused by virulent STEC strains results in bloody diarrhea, hemolytic uremic syndrome, and renal failure. According to the CDC, national enteric disease surveillance report E. coli O103 and O121 are responsible for 15.6% and 4.7% of total STEC cases, respectively. Previously, single nucleotide polymorphism (SNP) in the serogroup specific 0-antigen gene cluster has been identified and associated with virulent properties of STEC serogroup (Norman et al., 2012).

Purpose: Disclosed herein is a real-time PCR high-resolution melting assay for the specific detection of E. coli O103 and O121 strains. Further, the high-resolution melt profile is used for the differentiation of potentially virulent strains E. coli O103 and O121 from avirulent strains.

Methods: Serogroup-specific primers targeting the E. coli O103 (wbtD and wbtE genes) and O121 (vioA gene) were designed. A high-resolution melting real-time PCR assay was standardized. The developed for specific identification of O103 and O121 serogroups. The absolute quantification data from the assay was used for the serogroup identification, whereas the grouping obtained after high-resolution melt was used for the discrimination of potentially virulent strains from the avirulent strains. The assay was validated using 92 pure culture bacterial DNA samples. Assay were further validated using DNA isolated from enriched beef (n=36) and pork (n=36) samples collected by USDA FSIS.

Results: Both assays when validated using 92 pure culture bacterial DNA samples showed 100% specificity towards their serogroup. High-resolution melt data from the pure culture DNA samples showed the potentially virulent strains of E. coli O103 and O121 forming a distinct melt profile in the normalized and differential melting curves plots. Based on these specific melt profiles potentially virulent strains can be differentiated from the avirulent strains.

The assay accurately identified all avirulent O121 strains (n=33/72) from the enriched beef and pork samples. The E. coli O103 assay identified 17 positive samples among enriched beef and pork samples. The data from the two HRM assay for E. coli O103 and O121 was in agreement with the USDA data.

Significance: The assay developed in this study for specific identification O103 and O121 serogroup and differentiation of positive isolates into potentially virulent and avirulent strains can be used to improve food safety and reduce time needed by regulatory agencies for testing food samples.

TABLE 3
E. coli Isolates Used for the development and validation of assays:
Path-mlg-PCR

| Isolate | O group | stx mlg | ecf | espK1 | nleB | eae | F/sub |
|---|---|---|---|---|---|---|---|
| 16.2 | 26 | 0 | 0 | 0 | 0 | 0 | 0 |
| 222.1 | 26 | 0 | | 0 | 0 | 0 | 0 |
| 766.1 | 26 | 0 | 0 | 0 | 0 | 0 | 0 |
| 946.1 | 26 | 0 | 0 | 0 | 0 | 0 | 0 |
| 859.V3 | 26 | 0 | 0 | 0 | 0 | 0 | 0 |
| 3.5 | 45 | 0 | 0 | 0 | 0 | 0 | 0 |
| 151.1 | 45 | 0 | | 0 | 0 | 0 | 0 |
| 235.1 | 45 | 0 | 0 | 0 | 0 | 0 | 0 |
| 623.3 | 45 | 0 | 0 | 0 | 0 | 0 | 0 |
| 786.1 | 45 | 0 | 0 | 0 | 0 | 0 | 0 |
| 33 | 103 | 0 | | 0 | 0 | 0 | 0 |
| 75.2 | 103 | 0 | 0 | 0 | 0 | 0 | 0 |
| 302.1 | 103 | 0 | 0 | 0 | 0 | 0 | 0 |
| 621.2 | 103 | 0 | 0 | 0 | 0 | 0 | 0 |
| 802.1 | 103 | 0 | 0 | 0 | 0 | 0 | 0 |
| 739.3 | 111 | 0 | 0 | 0 | 0 | 0 | 0 |
| 75.3 | 121 | 0 | 0 | 0 | 0 | 0 | 0 |
| 219.5 | 121 | 0 | 0 | 0 | 0 | 0 | 0 |
| 508.3 | 121 | 0 | 0 | 0 | 0 | 0 | 0 |
| 745.1 | 121 | 0 | 0 | 0 | 0 | 0 | 0 |
| 967.1 | 121 | 0 | 0 | 0 | 0 | 0 | 0 |
| 219.7 | 145 | 0 | 0 | 0 | 0 | 0 | 0 |
| 317.2 | 145 | 0 | 0 | 0 | 0 | 0 | 0 |
| 221.2 | 0157 | 0 | | 0 | 0 | 0 | 0 |
| 645.V1 | O157 | 0 | 0 | 0 | 0 | 0 | 0 |
| 766.V1 | O157 | 0 | 0 | 0 | 0 | 0 | 0 |
| 999.V1 | O157 | 0 | 0 | 0 | 0 | 0 | 0 |
| hSTEC-03 | O26 | | | | | | |
| Imp 133.1 | O26 | | | | | | |
| May 063 | O26 | | | | | | |
| (USMARC_GB_STEC021) | | | | | | | |
| C4-462-2_7095 | O111 | | | | | | |
| F-A 790.1 | O111 | | | | | | |
| hSTEC 08 | O111 | | | | | | |

TABLE 4
Avirulent isolates of the top 7 STEC serogroups and HRM assay results

| | | | | | | | | O26 Assay | | O111 Assay | |
|---|---|---|---|---|---|---|---|---|---|---|---|
| | | Virulence factors present^{a} | | | | | | results | | results | |
| | O | | | | | | sub | | Vir/ | | Vir/ |
| Isolate | group | stx | espK1 | nleB | eae | nleF | AB | PCR | Avir | PCR | Avir |
| 16.2 | O26 | − | − | − | − | − | − | + | Avir | − | − |
| 97.1 | O26 | − | + | + | + | + | − | + | Vir | − | − |
| 699.1 | O26 | − | − | + | + | + | − | + | Avir | − | − |
| 859.V3 | O26 | − | − | − | − | − | − | + | Avir | − | − |
| 946.1 | O26 | − | − | − | − | − | − | + | Avir | − | − |
| hSTEC-03 | O26 | + | − | − | + | − | − | + | Vir | − | − |
| Imp 133.1 | O26 | + | − | − | + | − | − | + | Vir | − | − |
| May063 | O26 | + | − | − | + | − | − | + | Vir | − | − |
| 219.1 | O45 | − | + | + | + | + | − | − | − | − | − |
| 151.1 | O45 | − | − | − | − | − | − | − | − | − | − |
| 235.1 | O45 | − | − | − | − | − | − | − | − | − | − |
| 623.3 | O45 | − | − | − | − | − | − | − | − | − | − |
| 978.1 | O45 | − | + | + | + | + | − | − | − | − | − |
| 33 | O103 | − | − | − | − | − | − | − | − | − | − |
| 75.2 | O103 | − | − | − | − | − | − | − | − | − | − |
| 302.1 | O103 | − | − | − | − | − | − | − | − | − | − |
| 612.1 | O103 | | + | | + | | | | | | |
| 621.2 | O103 | − | − | − | − | − | − | − | − | − | − |
| 802.1 | O103 | − | − | − | − | − | − | − | − | − | − |
| 739.3 | O111 | − | − | − | − | − | − | − | − | + | Avir |
| C4-462- | O111 | + | − | − | + | − | − | − | − | + | Vir |
| 2_7095 | | | | | | | | | | | |
| F-A790.1 | O111 | + | − | − | + | − | − | − | − | + | Vir |
| hSTEC08 | O111 | + | − | − | + | − | − | − | − | + | Vir |
| 2014-5-4C | O111 | + | | | | | | | | + | Vir |
| 2016-11- | O111 | − | | | | | | | | + | Avir |
| 378B2 | | | | | | | | | | | |
| 75.3 | O121 | − | − | − | − | − | − | − | − | − | − |
| 219.5 | O121 | − | − | − | − | − | − | − | − | − | − |
| 508.3 | O121 | − | − | − | − | − | − | − | − | − | − |
| 745.1 | O121 | − | − | − | − | − | − | − | − | − | − |
| 785.2 | O121 | + | − | − | − | − | − | − | − | − | − |
| 967.1 | O121 | − | − | − | − | − | − | − | − | − | − |
| 219.7 | O145 | − | − | − | − | − | − | − | − | − | − |
| 317.2 | O145 | − | − | − | − | − | − | − | − | − | − |
| 766.1 | O157 | − | − | − | − | − | − | − | − | − | − |
| 221.2 | O157 | − | − | − | − | − | − | − | − | − | − |
| 645.V1 | O157 | − | − | − | − | − | − | − | − | − | − |
| 999.V1 | O157 | − | − | − | − | − | − | − | − | − | − |

^{a}Virulence genes included stx (universal primer set) eae, espK1, nleB, and nleF (Stromberg 2016) to identify avirulent E. coli of the 7 most common STEC serogroups.

### DNA Sample List

DNA of other bacterial strains used for the assay validation

1. Acinetobacter pittii

2. Citrobacter freundii

3. Enterobacter sp.

4. Enterobacter sp.

5. Acinetobacter pittii

6. Acinetobacter sp.

7. Acinetobacter pitti

8. Acinetobacter sp.

9. Acinetobacter calcoaceticus

10. Citrobacter freundii

11. Vibrio parahaemolyticus

12. Morganella morganii

13. Citrobacter sp.

14. Hafnia paralvei

15. Serratia marcescens

16. Serratia marcescens

17. Enterobacter cloacae

18. Enterobacter cloacae

19. Klebsiella pneumoniae

20. Vibrio parahaemolyticus

21. Citrobacter sp.

22. Enterobacter faecalis

23. Lelliottia amnigena

24. Lelliottia amnigena

25. Proteus mirabilis

26. Proteus mirabilis

27. Vibrio alginolyticus

28. Vibrio alginolyticus

29. Morganella morganii

30. Proteus mirabilis

31. Morganella morganii

32. Aeromonas hydrophila

33. Alcaligenes faecalis

34. Proteus mirabilis

35. Enterobacter hormaechei

36. Enterobacter hormaechei

37. Acinetobacter schindleri

38. Acinetobacter schindleri

39. Stenotrophomonas maltophilia

40. Stenotrophomonas maltophilia

### EXAMPLE 3: High-Resolution Melting Assays for the Detection of Virulent Strains of Escherichia coli O103 and O121

Shiga toxin-producing Escherichia coli (STEC) serogroups O103 and O121 are considered adulterants found in red-meat products. Two high-resolution melting assays were standardized to distinguish between potentially virulent and avirulent linage of E. coli O103 and O121 strains. The standardized assay was validated using 87 pure culture bacterial DNA samples, naturally contaminated beef (n=84) and pork (n=84) samples collected from the USDA surveillance program, and inoculated beef and spinach samples (n=36). The HRM assays were able to specifically identify the O103 and O121 strains and differentiation between the potentially avirulent or virulent STEC lineages. Data from this study showed greater than percent sensitivity and specificity for the O103 and O121 assays developed in this study.

### Materials and Methods

### Primer Design

For this study, the serogroup specific primer-pairs were designed using the Primer3 software (Untergasser et al., 2012). The primer pairs used for the specific identification of O103 were intended to target the single nucleotide polymorphism (SNP) located in the wbtD gene at the 937 position (C to T). In comparison, the O121 serogroups were designed to target the SNP in the vioA gene located at the 313 position (C to T). These SNPs have been previously associated with the differentiation of potentially virulent and avirulent strains (Norman et al., 2012). Designed primer-pairs were tested for their specificity and their potential to cross-amplification other serogroups with the NCBI Primer-BLAST tool. The designed oligonucleotides were synthesized by IDT (Coralville, Iowa, USA). All the oligonucleotides used for this study are listed in Table 5.

TABLE 5
Oligonucleotides for the specific detection
of E. coli O103 and O121 by the HRM assays.

| | | Target | Product |
|---|---|---|---|
| Name | Primer Sequence | Gene | Size |
| O103- | 5′-GTAAATGGCGCGAAGCAATA-3′ | wbtD | 146 |
| 863F | (SEQ ID NO: 7) | | |
| O103- | 5′-TTGTTGGTTAATGCATGACTCT-3′ | — | |
| 1079R | (SEQ ID NO: 8) | | |
| O121- | 5′-TTTGGGTGTTAAAGGCGAAG-3′ | vioA | 98 |
| 210F | (SEQ ID NO: 9) | | |
| O121- | 5 ′-CGCCTCTTCAATTCTTCTCG-3′ | | |
| 354R | (SEQ ID NO: 10) | | |

### Real-time PCR

The HRM assays were performed on a LightCycler® 96 real-time PCR instrument (Roche Diagnostics Corp., Indianapolis, USA). A 2x LightCycler® 480 High-Resolution Melting Master (Roche Diagnostics Corp., Indianapolis, USA) was used for the O121 assay. Whereas Apex qPCR 2× GREEN master mix (Apex Bioresearch, N.C., USA) supplemented with 0.25 μL of lx EvaGreen (Biotium Inc., Hayward, Calif., USA) and 1mM MgCl₂ was used for the O103 assay. A typical 10 μl reaction mixture consisted of 20 ng of DNA, 1 μM of forward and reverse primer-pair, 3.0 mM MgCl2, and 0.6 μL of nuclease-free water. A three-step PCR protocol was used for the amplification of the target sequence. The amplification protocol entailed for the O121 assay using 2× LightCycler® 480 High-Resolution Melting Master consisted of an initial denaturation step at 95 ° C. for 600s, followed by 40 cycles of denaturation at 95 ° C. for 15s, with annealing at 60 ° C. for 30s, and extension at 72 ° C. for 10s. Whereas the amplification for the O103 assay was performed with an initial denaturation step at 95 ° C. for 900s, followed by 40 cycles of denaturation at 95 ° C. for 15s, with annealing at 60 ° C. for 30s, and extension at 72 ° C. for 25s. The HRM step for both assays consisted of a gradual temperature increase of 0.07 ° C/s from 65 to 97 ° C. Fluorescence data from the amplification and HRM steps were collected in channel 1 of the instrument. The HRM data for O103 was analyzed with a pre-melt region of 71.9-72.9 ° C. and a post-melt region of 76.3-77.3 ° C., while the HRM for O121 was performed with a pre-melt region of 75.5-76.5° C. and a post-melt region of 79.8-80.8° C. Additionally for HRM analysis all negative samples were removed from the analysis.

### Bacterial Strains

The specificity of the two HRM assays standardized in this study was validated using DNA isolated from 87 pure bacteria strains (Table 8) as described in Singh et al. 2020.

Assay Validation with Inoculated Food Samples

The performance of the two assays were validated by inoculating the food samples with O103 and O121 strains. Six strains of O103 and O121 were chosen for the inoculation of food samples (Table 1). Each of the strains was individually cultured overnight in 10 mL of tryptic soy broth (TSB) (Hardy diagnostics, Santa Maria, Calif., USA). After the incubation, the cultures were serially diluted and spread plated on tryptic soy agar (TSA) (Hardy diagnostics, Santa Maria, Calif., USA). The cultures awaiting enumeration were kept in the refrigerator at 4 ° C., while the PCA plates were incubated at 37 ° C. overnight. Count from the PCA plates were used to calculate the appropriate dilution and volume to achieve 10 CFU. The computed volume was used to spike the food samples. Additionally, the inoculum was spread plated onto TSA agar to accurately enumerate the inoculation load use to spiked into the food samples.

TABLE 6
E. coli strains used for inoculating food samples

| E. coli Strain | Source | Virulence Gene |
|---|---|---|
| O103 33 | Beef | stx^{-ve}, eae^{-ve} |
| O103 75.2 | Beef | stx^{-ve}, eae^{-ve} |
| O103 302.1 | Beef | stx^{-ve}, eae^{-ve} |
| O103-1 | Human | stx₁, eae |
| O103-2 | Beef | stx₁, eae |
| O103-3 | Beef | stx₁, eae, |
| O121 75.3 | Beef | stx^{+ve}, eae^{-ve} |
| O121 219.55 | Beef | stx^{-ve}, eae^{-ve} |
| O121 508.3 | Beef | stx^{-ve}, eae^{-ve} |
| O121-2 | Human | stx, eae |
| O121-3 | Beef | stx, eae |
| O121-4 | Beef | stx, eae |

### Food Sample Preparation

Ground beef (12% fat 88% lean), beef roast, and spinach were purchased from the local grocery store (Tallahassee, Florida). The beef roast was thinly sliced according to the USDA N60 sampling procedure (USDA 2008). Twenty-five grams of spinach, 325 g of the ground beef, and beef trims were all separately transferred into sterile Whirl-Pak® filter stomacher bags (Nasco, Fort Atkinson, Wis.). The food samples were individually spiked with one of the 12 strains of E. coli O103 and O121 at 10 CFU per bag. The food samples were incubated for 15 minutes at room temperature to facilitate attachment of the inoculum to the food matrix. The inoculated food samples were stored at 4 ° C. for 24 hours to stress the inoculum. At the end of the 24-hour stressing period, the food samples were diluted with a modified TSB (mTSB) with casamino acid (10 g/L) and 2mg/L of novobiocin. The 325 g bags of ground beef and beef trims were diluted with 975 ml of the mTSB, while 25 g bags of spinach samples were diluted with 225 ml of the mTSB. The beef bags were lightly massaged after the addition of enrichment broth, whereas the spinach bags were stomached at 230 rpm for 2 minutes. The food samples with enrichment broth were incubated for a 15-hour enrichment at 42 ° C. After 15 hours, 1.8 mL samples were taken from the enrichment bags and transferred to Eppendorf tubes. DNA from the 1.8 mL of enrichments were isolated using the DNeasy® Power Food Microbial Kit (Qiagen, Hilden, Germany) following the manufacturer's instructions.

### Results

Two HRM assays for the specific detection of E. coli O103 and O121 serogroups were standardized. The O103 and O121 primer-pairs at first were standardized with pure culture DNA samples. The assay was accurately able to identify all the O103 strains (33, 75.2, 302.1, 612.1 621.2, 745.1, 802.1, 103-1, 103-2, 103-3) tested in the study. The O103 primer-pair did not show any cross-amplification with any other 77 non-target bacteria strains. The O103 primer-pair showed 100% sensitivity and specificity for the specific detection of E. coli O103 serogroup. Further, the HRM analysis of samples generating positive amplification correctly differentiated between potentially virulent and avirulent strains lacking crucial virulence genes (FIGS. 5a and 5b).

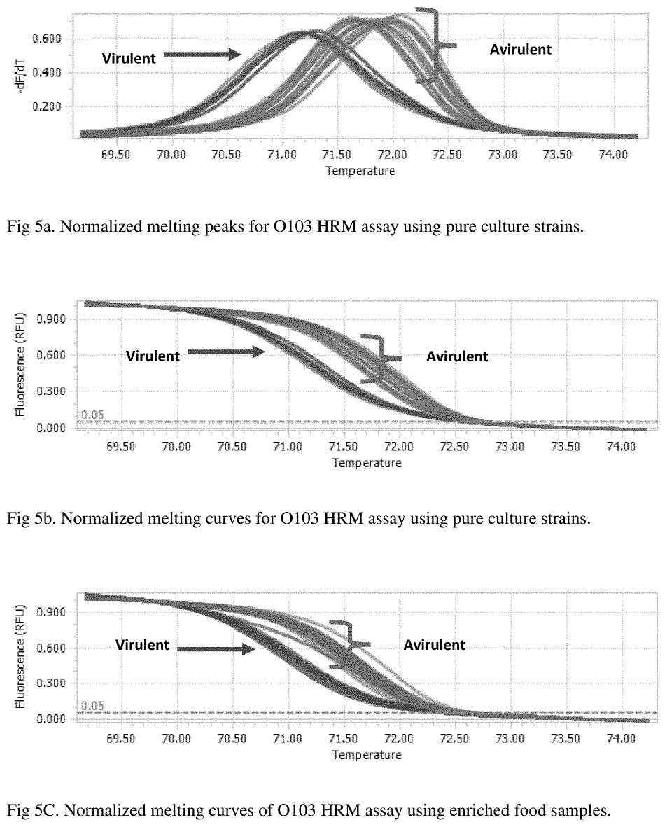

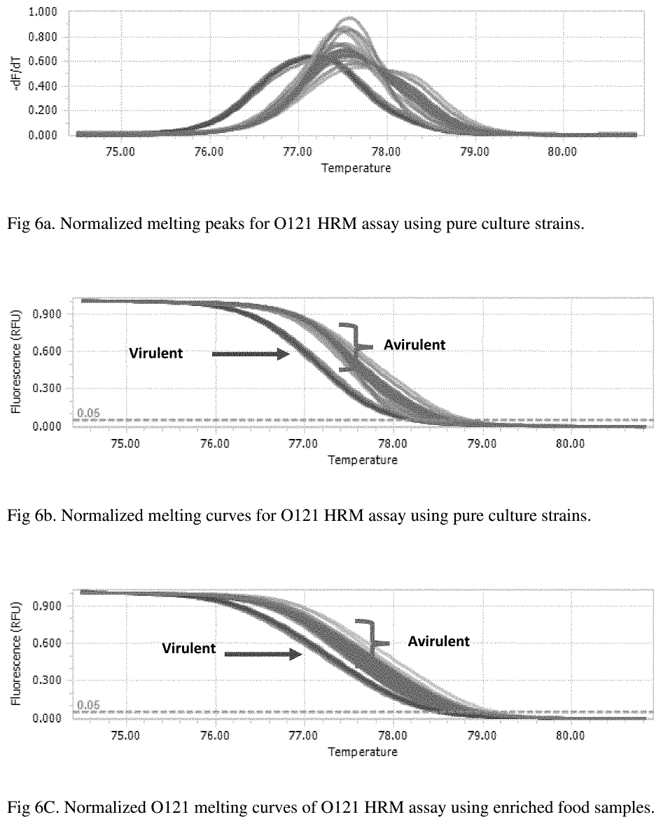

Similarly, the O121 HRM assay correctly identified all ten O121 isolates (2′ nph1_12738, 211-1, 219.5, 256-1, C4-63-1_3218, V2-G2 1-C 16.3, 508.3, 75.3, 785.2 and 967.1). The O121 HRM analysis of the positive samples was able to show the distinction between the melting plots for the virulent and avirulent strains (FIG. 6a and b). The O121 primer-pair did not show any non-specific amplification with any of the other 77 non-target bacteria strains showed 100% sensitivity and specificity with the pure culture strains.

The food samples inoculums used for the spiking food samples ranged from 2 to 11 CFU. The two HRM assays standardized in this study were able to correctly identify the target E. coli strains (O103 33, 103 75.2, 103 302.1, 103-1, 103-2, 103-3, 121 75.3, 121 219.5, 121 508.3, 2′ nph1_12738, C4-63-1_3218, V2-G2 1-C 16.3) following a 15 h enrichment period. Further, the HRM plots accurately distinguished the virulent and avirulent strains (FIGS. 5c and 6c).

### 4. Discussion

The current USDA, FSIS standard method (MLGS) initially screens samples for the presence of stx and eae virulence genes. Samples testing positive for these two virulence genes are further screened for presence of seven E. coli serogroups which are considered adulterant. The MLGS assays in total employs ten primer pairs and 10 dual-labeled probes in a multiplex format for detection of seven STEC serogroups (i.e., O157, O26, O45, O111, O103, O121, and O145). The stx and eae genes are founds in other Gram-negative genera (Margot et al., 2013; Quiros et al., 2015; Gassama et al., 2001; Hyma et al., 2005). This testing approach often results in higher screen positive (stx⁺, eae⁺) and potentially positive (stx⁺, eae⁺, O-group⁺) rates (Bosilevac and Koohmaraie, 2012). The reason for this big difference in the potentially positive samples and confirmed STEC strains is likely due to positive results generated by presence of non-pathogenic (stx⁻, eae⁻) strains of the big-seven serogroups.

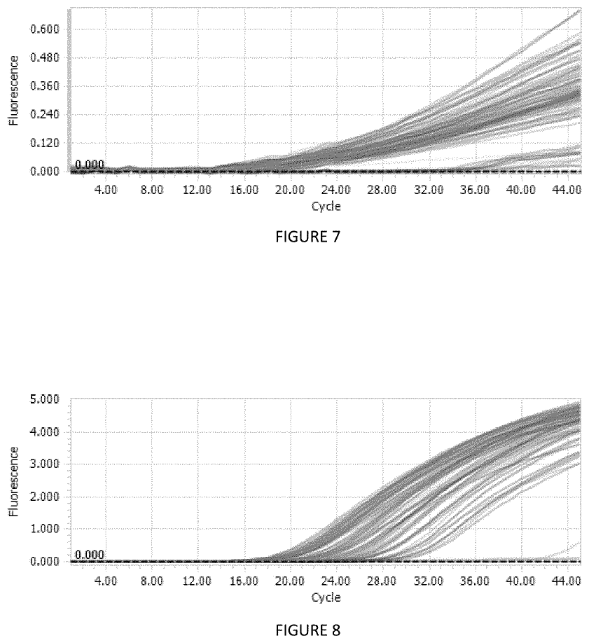

The initial goal was to standardize both assays using one high-resolution master mix (i.e., 2× LightCycler® 480 High-Resolution Melting Master). The master mix generated very distinct melt profile for the accurate identification of target SNPs. However, the O103 primer-pair generated poor amplification plots (FIG. 7) in the absolute quantification, making it difficult to call a sample positive or negative based on the Cq values. Initial attempts to optimize the O103 PCR reaction by optimizing primer concentration, reaction conditions, MgCl₂ concentrations, primer with locked nucleic acid bases, different HRM master mixes did not result in any significant improvement in the amplification curve. The use of Apex qPCR 2× GREEN master mix supplemented with 0.25 μL of lx EvaGreen and 1mM MgCl₂ was the only combination which generated a sigmoid amplification plots and acceptable high-resolution melting plots. Even though the Apex qPCR 2× GREEN master mix is not a HRM master mix, studies conducted in the laboratory have shown its suitability for the HRM assay (Laxmi Sharma et al., 2020). Further incorporation of EvaGreen and optimization of MgC12 to the master mix further improved its ability to identify SNP.

### Conclusion

Compared to commonly used approach that relies on testing for stx, eae, and serogroup specific genes, a novel approach for the specific identification of potentially virulent strains of O103 and O121 serogroups has been standardized. Application of this approach will help to decrease false-positive rate due to presence of avirulent strains, reduce burden of confirming avirulent strains by regulatory labs and lessen product loss for the stake holders.

### EXAMPLE 4: Multiplex Dual-Labeled 5′-Nuclease Assays for the Detection of Virulent Strains of Escherichia coli O26, O111, O103 and O121

Dual-labeled 5′-nuclease assays are considered as a gold standard for the diagnostic assay. A 5′-nuclease assays relies on specific binding of a probe sequence to the target region and thus are more specific than intercalating dye based real-time PCR chemistries. Due to higher target specificity use of probe-based assay is recommended by all major regulatory agencies.

In this study eight dual-labeled probes (Table 7) were designed for the specific identification of potentially virulent and avirulent strains of E. coli O26, O111, O103 and O121. The serogroup specific gene target with the SNP was amplified using pre-validated primer-pairs used for the high-resolution melting assays (Table 7). Dual-labeled probes were designed using the Primer3 software (Untergasser et al., 2012). The central three to six bases of the probe sequence flanking the target SNP was replaced by locked nucleic acid (LNA) bases. The LNA bases have excellent ability for discriminating DNA sequences differing by a single base difference, are considered superior to DNA based probes. All probe targeting the potentially virulent genotype were labeled at 3′ end with the 6-FAM dye and probes targeting the potentially avirulent genotype were labeled with the HEX dye. The USDA FSIS recommended primer and probe sequence targeting the bacterial 16 rRNA gene sequence was used as an IAC (MLG 5C Appendix 4.00).

A multiplex 5′-nuclease assays was standardized for the specific detection of potentially virulent and avirulent strains of E. coli O26, O111, O103 and O121. Each multiplex real-time PCR reaction consisted of serogroup primer pair, two dual-labeled probes, primer amplifying the bacterial 16 rRNA gene sequence and probe detecting the 16 rRNA gene sequence. Multiplex reaction 1 consisted of SEQ ID NO: 1, SEQ ID NO: 2, SEQ ID NO: 11, SEQ ID NO: 12, SEQ ID NO: 19, SEQ ID NO: 20 and SEQ ID NO: 21. Multiplex reaction 2 consisted of SEQ ID NO: 3, SEQ ID NO: 4, SEQ ID NO: 13, SEQ ID NO: 14, SEQ ID NO: 19, SEQ ID NO: 20 and SEQ ID NO: 21. Multiplex reaction 3 consisted of SEQ ID NO: 7, SEQ ID NO: 8, SEQ ID NO: 15, SEQ ID NO: 16, SEQ ID NO: 19, SEQ ID NO: 20 and SEQ ID NO: 21. Multiplex reaction 4 consisted of SEQ ID

NO: 9, SEQ ID NO: 10, SEQ ID NO: 17, SEQ ID NO: 18, SEQ ID NO: 19, SEQ ID NO: 20 and SEQ ID NO: 21.

Real-time PCR assays were performed on LightCycler® 96 real-time PCR instrument (Roche Diagnostics Corp., Indianapolis, USA) with 2x Apex qPCR Probe Master Mix (without ROX) (Apex Bio Research Products, N.C., USA). A 10 μl real-time PCR reaction mixture consisted of 20 ng of DNA, 500 nM of E. coli O26, O111, O103 and O121 forwards and reverse primers (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8), 50 nM of probe (SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18), 100 nM of 16S primer (SEQ ID NO: 19, SEQ ID NO: 20), and 50 nM of 16S probe (SEQ ID NO: 21). A three-step PCR protocol was used for the amplification of the target sequence. The amplification protocol for O26, O111, and O121 consisted of an initial denaturation step at 95 ° C. for 900 seconds, 45 cycles of 95 ° C. for 20 s, 63 ° C. for 20 s and 72 ° C. for 5 s. Whereas, the amplification protocol for O103 consisted of an initial denaturation step at 95 ° C. for 900 seconds, 45 cycles of 95 ° C. for 20 s, 55 ° C. for 20 s and 72 ° C. for 5 s. Amplification data were collected in the 6-FAM, HEX and CY5 detection channels of the instrument.

TABLE 7
PRIMER AND DUAL-LABELED PROBES

| | | | |
|---|---|---|---|
| | | Target | Product |
| Name | Primer Sequence | Gene | Size |

E. coli O26

| | | | |
|---|---|---|---|
| O26-32F | 5′-GTG GCA CTG GTT CTT TTG GT-3′ | fnl1 | 87 |
| | (SEQ ID NO: 1) | | |
| O26-219R | 5′-ACCACGCGTTGCATTTAGAA-3′ | | |
| | (SEQ ID NO: 2) | | |
| O26-Vir- | 5′-6-FAM/CA GAT A+T+T +A+C+T | | |
| Probe | +GAA ATA CG-3′-IABkFQ (SEQ ID | | |
| | NO: 11) | | |
| O26-AVir- | 5′-HEX/ACA +GA+T AT+T +G+CT | | |
| Probe | +GAA-3′-IABkFQ (SEQ ID NO: 12) | | |

E. coli O111

| | | | |
|---|---|---|---|
| O111-634F | 5′-CTT CGA GCT CAT GGT TGG AC- | wbdK | 84 |
| | 3′ (SEQ ID NO: 3) | | |
| O111-717R | 5′-CGA CTC TTC GAA AAT ATC ATC | | |
| | A-3′(SEQ ID NO: 4) | | |
| O111-Avir- | 5′-HEX/GT+T A+CA G+G+C +ACT | | |
| Probe | +AAG-3′-IABkFQ | | |
| | (SEQ ID NO: 13) | | |
| O111-Vir- | 5′-6-FAM/GGT+T A+CA G+G+T | | |
| Probe | +ACT +AAG-3′-IABkFQ (SEQ ID NO: | | |
| | 14) | | |

E. coli O103

| | | | |
|---|---|---|---|
| O103-863F | 5′-GTAAATGGCGCGAAGCAATA-3′ | wbtD | 146 |
| | (SEQ ID NO: 7) | | |
| O103-1079R | 5′-TTGTTGGTTAATGCATGACTCT-3′ | | |
| | (SEQ ID NO: 8) | | |
| O103-Avir- | 5′-HEX/AT TAA C+C+T +G+C+A +TAT | | |
| Probe | TAA A-3′-IABkFQ (SEQ ID NO: 15) | | |
| O103-Vir- | 5′-6-FAM/AT TAA C+C+T +G+T+A | | |
| Probe | +TAT TAA A-3′-IABkFQ (SEQ ID NO: | | |
| | 16) | | |
| Wzx O103-F | 5′-TTG GAG CGT TAA CTG GAC CT- | | |
| | 3′ (SEQ ID NO: 22) | | |
| Wzx O103-R | 5′-ATA TTC GCT ATA TCT TCT TGC | | |
| | GGC-3′ (SEQ ID NO: 23) | | |
| Wzx O103-P | 5′ CAL Fluor Red 610-AGG CTT ATC | | |
| | TGG CTG TTC TTA CTA CGG C-BHQ2-3′ | | |
| | (SEQ ID NO: 24) | | |
| O103:H25-F: | GAAACAAACGGTAAATGGCG (SEQ | | |
| | ID NO: 25) | | |
| O103:H25-R: | TGTTGGTTAATGCATGACTCT (SEQ | | |
| | ID NO: 26) | | |
| P1: | AGA+A+T+ATTAACCT+G+C+ATATTA | | |
| | (SEQ ID NO: 27) | | |
| P2: | AGA+A+C+ATTAACCT+G+C+ATATTA | | |
| | (SEQ ID NO: 28) | | |

E. coli O121

| | | | |
|---|---|---|---|
| O121-210F | 5′-TTTGGGTGTTAAAGGCGAAG-3′ | vioA | 98 |
| | (SEQ ID NO: 9) | | |
| O121-354R | 5′-CGCCTCTTCAATTCTTCTCG-3′ | | |
| | (SEQ ID NO: 10) | | |
| O121-Avir- | 5′-HEX/CG ATA TTG A+T+C +CCA | | |
| Probe | AAA CC-3′-IABkFQ (SEQ ID NO: 17) | | |
| O121-Vir- | 5′-6-FAM/CG ATA TTG A+T+T +CCA | | |
| Probe | AAA CC-3′-IABkFQ (SEQ ID NO: 18) | | |

16S RNA IAC

| | | | |
|---|---|---|---|
| 16SRna-F | CCT CTT GCC ATC GGA TGT G (SEQ | 16S | 99 |
| | ID NO: 19) | | |
| 16SRna-R | GGC TGG TCA TCC TCT CAG ACC | | |
| | (SEQ ID NO: 20) | | |
| 16S rRNA | 5′-Cy5-GTG GGG TAA CGG CTC ACC | | |
| Probe | TAG GCG AC-3′-IBRQ (SEQ ID NO: | | |
| | 21) | | |

TABLE 8
List of pure culture strains used for testing
specificity of the O103 and O121 HRM assays.

Acinetobacter pittii 27B MM
Acinetobacter schindleri 27B MC1
Acinetobacter schindleri 27B MC2
Aeromonas hydrophila 20A MM2
Alcaligenes faecalis 22B MC2 1
Bifidobacterium bifidum 11863
Citrobacter freundii 14B MC1
Citrobacter freundii 16B MM1
E. coli 25922
E. coli O103-1 hSTEC 05
E. coli O103-2 MDR 0089 (USMARC_GB_STEC 045)
E. coli O103-3 Mar 125B (USMARC_GB_STEC 046)
E. coli O103 302.1
E. coli O103 33
E. coli O103 612.1
E. coli O103 621.2
E. coli O103 745.1
E. coli O103 75.2
E. coli O103 802.1
E. coli O111 C4-462-2_7095
E. coli O111 F-A 790.1
E. coli O111:H8- hSTEC 08
E. coli O111 739.3
E. coli O121 nphl_12738
E. coli O121 211-1
E. coli O121 219.5
E. coli O121 256-1
E. coli O121 C4-63-1_3218
E. coli O121 V2-G2 1-C 16.3
E. coli O121 508.3
E. coli O121 75.3
E. coli O121 785.2
E. coli O121 967.1
E. coli O145 1 hSTEC 22
E. coli O145 170-2
E. coli O145 2 May 109
E. coli O145 219.7
E. coli O145 3 C4-69-1_3275
E. coli O145 317.2
E. coli O145 56.2
E. coli O145 690.1
E. coli O157 221.2
E. coli O157 645.V1
E. coli O157 766.V1
E. coli O157 E0018
E. coli O26 16.2
E. coli O26 699.1
E. coli O26 859.V3
E. coli O26 946.1
E. coli O26 97.1
E. coli O26 hSTEC-03
E. coli O26 Imp 133.1
E. coli O26 May 063 (USMARC_GB_STEC 021)
E. coli O45 151.1
E. coli O45 219.1
E. coli O45 235.1
E. coli O45 623.3
E. coli O45 978.1
E. coli 14B MM
E. coli 27A MNC
Enterobacter hormaechei 25B MC1
Enterobacter hormaechei 25B MC2
Enterobacter lignolyticus 15B MC2 -1
Enterobacter lignolyticus 15B MM1
Enterococcus faecalis 15A MNC1
Enterococcus faecalis 15B MC2 2
Klebsiella pneumoniae 24A MNC2
Lactobacillus plantarum
Lelliottia amnigena 15B MC1 1
Morganella morganii 15A MNC2
Morganella morganii 16B MC1 1
Obesumbacterium proteus 17B MM2
Obesumbacterium proteus 28A MNC
Pantoea alhagi 25A MNC
Proteus mirabilis 16B MC1-2
Proteus mirabilis 16B MC2 1
Providencia alcalifaciens 17B MC2-2
Salmonella Typhimurium 14028
Serratia marcescens 28B MC2
Serratia marcescens 28B MM
Stenotrophomonas maltophilia 29B MC1
Stenotrophomonas maltophilia 29B MC2
Vibrio alginolyticus 18B MC2-1
Vibrio alginolyticus 18B MM
Vibrio fluvialis 24B MM
Vibrio parahaemolyticus 24B MC1
Vibrio parahaemolyticus 24B MC2

### EXAMPLE 5: Improvements to Original Assay

### O26 Multiplex 5′-Nuclease Assay:

The initial O26-primer O26-219R (SEQ ID NO: 2) was replaced: The initial reverse primer had multiple repeat sequences and was causing non-specific amplification in some samples. The reverse primer was replaced to fix the non-specific amplification.

The initial O26-AVir-Probe (SEQ ID NO: 12) sequences showed a fluorescence signal bleed-through problem. The signal from HEX was getting detected in the FAM channel, interfering with the assay.

The probe sequences were redesigned to solve the bleed-through problem. Additionally, the LNA bases were relocated to improve the SNP detection. The new probes have superior SNP detection capability.

O111 Multiplex 5′-nuclease assay:

The initial probe sequences O111-Avir-Probe (SEQ ID NO: 13) and O111-Vir-Probe (SEQ ID NO: 14) showed a fluorescence signal bleed-through problem.

The probe sequences were redesigned to solve the bleed-through problem. Additionally, probe sequences were cut shorter, and the LNA bases were relocated to improve the SNP detection capability. The new probes showed superior SNP detection capability.

O103 Multiplex 5′-nuclease assay:

The initial primer pair for the amplification of O103 (SEQ ID NO: 7); and O103 — 995R: (SEQ ID NO: 8) showed poor amplification efficiency. To improve the PCR amplification efficiency, the new primer pair was designed. The new primer pair showed improved PCR amplification. This set of primer pairs in association with the probes (SEQ ID NO: 15 and SEQ ID NO: 16) can be used for the virulence determination.

To further improve the O103 detection capability of the multiplex assay, a new set of published and validated primer and probe were added to the assay (SEQ ID NOS: 22, 23, and 24). Signals from this set of primer pairs can be used for detecting the presence of O103 in the samples. Thus, all new changes greatly improved the O103 detection and differentiation of virulent and avirulent strains.

O121 Multiplex 5′-nuclease assay:

The O121-257F was replaced with the O121-vioA-210F (SEQ ID NO: 9). The original primer showed amplification with the non-template control. The change solved the non-specific amplification issues.

A new set of primers/probes was added:

O103:H25-F:
(SEQ ID NO: 25)
GAAACAAACGGTAAATGGCG
O103:H25-R:
(SEQ ID NO: 26)
TGTTGGTTAATGCATGACTCT
P1-Avir:
(SEQ ID NO: 27)
5′-HEX/AGA+A+T+ATTAACCT+G+C+ATATTA-3′-IABkFQ
P2-Vir:
(SEQ ID NO: 28)
5′-6-FAM/AGA+A+C+ATTAACCT+G+C+ATATTA IABkFQ

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

SEQUENCES
SEQ ID NO: 1:
O26 fnl1-32F (forward primer):
5′-GTG GCA CTG GTT CTT TTG GT-3′
(SEQ ID NO: 1)
SEQ ID NO: 2:
O26 fnl1-219R (reverse primer):
5′-ACCACGCGTTGCATTTAGAA-3′
(SEQ ID NO: 2)
SEQ ID NO: 3
O111 wbdK-634F (forward primer):
5′-CTT CGA GCT CAT GGT TGG AC-3′
(SEQ ID NO: 3)
SEQ ID NO: 4:
O111 wbdK-717R (reverse primer):
5′-CGA CTC TTC GAA AAT ATC A-3′
(SEQ ID NO: 4)
SEQ ID NO: 5:
AY763106.1: O26 Escherichia coli serotype
O26:H11 O-antigen operon, partial
sequence:
GCATTAATACCCCTATTAATCAACCTAAGAGCCGCTTATT
TCACAGCATGCTCTGAAGTAATATGGAATAATAAAGTGAA
GATACTTGTTACTGGTGGCGCAGGATTTATTGGTTCTGCT
GTAGTTCGTCACATTATAAATAATACGCAGGATAGTGTTG
TTAATGTCGATAAATTAACGTACGCCGGAAACCTGGAATC
ACTTGCTGATGTTTCTGATTCTGAACGCTATGTTTTTGAA
CATGCGGATATTTGCGATGCAGCTGCAATGGCGCGGATTT
TTGCTCAGCATCAGCCGGATGCAGTGATGCACCTGGCTGC
TGAAAGCCATGTTGACCGTTCAATTACCGGCCCTGCGGCA
TTTATTGAAACCAATATTGTTGGTACTTATGTCCTTTTGG
AAGCCGCTCGCAATTACTGGTCTGCTCTTGATAGCGACAA
GAAAAATAGCTTCCGTTTTCATCATATTTCTACTGACGAA
GTCTATGGTGATTTGCCTCATCCTGACGAAGTAAATAATA
CAGAAGAATTACCCTTATTTACTGAGACGACAGCTTACGC
GCCAAGCAGTCCTTATTCCGCATCCAAAGCATCTAGCGAT
CATTTAGTCCGCGCGTGGAAACGTACCTATGGTTTACCGA
CCATTGTGACTAACTGTTCGAATAACTACGGCCCTTATCA
CTTTCCGGAAAAATTGATTCCGCTGGTAATTCTTAATGCT
CTGGAAGGTAAGGCATTACCTATTTATGGCAAAGGGGATC
AAATTCGCGATTGGCTATATGTTGAAGATCATGCGCGTGC
GTTATATACCGTTGTCACCGAAGGTAAAGCGGGTGAAACT
TATAACATTGGCGGACACAACGAAAAGAAAAACATCGATG
TTGTGCTGACTATTTGTGATTTGTTGGATGAGATTGTACC
GAAAGAGCAATCTTATCGTGAGCAAATTACTTATGTTGCC
GATCGTCCGGGACACGATCGCCGTTATGCGATTGATGCTG
AGAAGATTAGCCGCGAATTGGGCTGGAAACCGCAGGAAAC
GTTTGAGAGCGGGATTCGGAAGACAGTGGAATGGTACCTG
TCCAATACAAAATGGGTCGAAAATGTGAAAAGTGGTGCCT
ATCAGTCATGGATTGCACAGAACTATGAGGGCCGTCAGTA
ATGAATATCCTCCTTTTCGGCAAAACAGGGCAGGTAGGTT
GGGAACTACAGCGTGCTCTGGCACCTCTGGGTAATTTGAT
TGCTCTTGATGTTCACTCTACTGATTATTGCGGTGATTTT
AGTAATCCTGAAGGTGTAGCTGAAACCGTAAGAAGCATTC
GGCCTGATATTATTGTCAACGCAGCCGCTCACACCGCAGT
AGACAAAGCAGAATCAGAACCGGAGTTTGCACAATTACTT
AACGCAACAAGTGTCGAAGCGATTGCGAAAGCAGCCAATG
AAGTCGGCGCCTGGCTTATTCATTACTCGACTGATTACGT
CTTCCCTGGAAATGGCGATATGCCATGGCGGGAGACGGAT
GCAACCGCACCACTAAATGTTTACGGTGAAACCAAGTTAG
CCGGAGAAAAAGCGTTACAGGAATATTGCGCAAAGCATCT
TATTTTCCGGACCAGCTGGGTCTATGCAGGAAAAGGAAAT
AACTTCGCCAAAACGATGTTACGTCTGGCAAAAGAGCGTG
AAGAATTAGCGGTTATTAATGATCAGTTTGGTGCGCCAAC
AGGTGCTGAACTGCTGGCTGATTGTACAGCACATGCCATT
CGTGTCGCACTGAATAAACCAGAAGTCGCAGGCTTGTACC
ATCTGGTAGCCAGTGGTACCACAACCTGGCACGATTATGC
TGCGCTGGTTTTTGAAGAGGCACGCAAAGCAGGTATTCCC
CTTGCACTCAACAAGCTCAACGCAGTACCAACAACAGCCT
ATCCTACACCAGCTCGTCGTCCGCATAACTCTCGCCTTAA
TACAGAAAAATTTCAGCAGAAATTTGCGCTTGTTTTGCCT
GATTGGCAGGTTGGCGTGAAACGAATGCTCAACGAATTAT
TTACGACTACAGCAATTTAATAGTTTTTGCATCTTGTTCG
TGATGGTGGAGCAAGATGAATTAAAAGGAATGATGAAATG
AAAACGCGTAAAGGTATTATTTTAGCTGGTGGTTCGGGTA
CTCGTCTTTATCCTGTAACTATGGCTGTCAGTAAACAGTT
GTTACCGATTTATGATAAACCGATGATCTATTACCCGTTG
TCTACACTGATGTTAGCGGGTCTTCGCGATATTCTGATTA
TTAGTACGCCACAGGATACTCCTCGTTTTCAACAACTGCT
GGGTGACGGGAGCCAGTGGGGGCTAAATCTTCAGTACAAA
GTGCAACCGAGTCCAGATGGTCTTGCGCAGGCATTTATCA
TCGGTGAAGAGTTTATCGGTGGTGATGATTGTGCTTTGGT
TCTAGGTGATAATATCTTTTACGGTCACGATCTGCCGAAG
TTAATGGATGTCGCTGTTAACAAAGAAAGTGGTGCAACGG
TATTTGCCTATCACGTTAATGATCCTGAACGCTACGGTGT
CGTTGAGTTTGATAAAAACGGTACGGCGATCAGCCTGGAA
GAAAAACCGCTACAACCAAAAAGTAATTATGCGGTAACCG
GGCTTTATTTTTATGATAACTACGTTGTCGAAATGGCGAA
AAATCTTAAGCCTTCTGCCCGCGGTGAACTGGAAATTACC
GATATTAACCGTATTTATATGGAACAGGGGCGTTTATCCG
TTGCCATGATGGGACGTGGTTATGCATGGCTGGACACGGG
GACACATCAAAGTCTTATTGAGGCAAGCAATTTTATCGCA
ACAATAGAAGAACGTCAGGGGCTGAAAGTTTCCTGCCCGG
AAGAAATTGCTTACCGTAAAGGGTTTATCGATGCTGAGCA
GGTGAAAGTATTAGCTGAACCGTTGAAGAAAAATGCTTAT
GGTCAGTATCTGCTGAAAATGATTAAAGGTTATTAATAAA
ATGAACGTAATTAAAACAGAAATTCCAGATGTACTGATTT
TTGAACCGAAAGTTTTTGGTGATGAGCGTGGTTTCTTTAT
GGAAAGCTTTAATCAGAAAGTTTTTGAAGAAGCTGTAGGG
CGGAAGGTTGAATTTGTTCAGGATAATCATTCTAAATCAT
CTAAGGGTGTGTTACGCGGACTGCACTATCAGCTGGAACC
CTATGCTCAAGGTAAATTAGTTCGTTGTGTGGTCGGTGAA
GTATTTGATGTAGCAGTTGATATTCGTAAATCGTCACCTA
CATTTGGGAAATGGGTTGGGGTGAATTTGTCTGCTGAGAA
TAAGCGTCAGTTGTGGATCCCTGAGGGGTTTGCGCATGGT
TTTTTGGTGTTAAGCGAGATAGCAGAGTTTGTTTATAAGA
CGACAAATTATTATCATCCTGAATCAGAAGGTTGCATAAA
ATGGGATGATTCTTTTTTGATGATTGATTGGCCAAATAAA
CCGATAGCAATCTCAGAAAAAGATAAAAAAGGCTCATCAA
TTTTGAGGCTAAATGACTATTGATGTTGAAAAAAAAACTT
CAAAAAATAAAGGAATATCATTCAGTATTGGAGTTGGCAA
TAATTCAGGGTGCGAATGCCATATTTCCTGTGTTGGTATT
CCCATTTTTTCTTATTACCTTAGGGGAAAACATCTTTTCA
AGTATTGCTGTTGGTGAAGTACTAGCACTATATGTGCTTA
TATTTTCGCTATACAGTTTTGATATTATAAGTGTGCAGAA
GGTAATTTCAAGTGTGACAAAAGATGAAATATTTAAAGTT
TACATTCTGACACTAATCTGTAGGTTGTGTTTATTTGTTA
TTTCAGGAATATGTCTTTTATTTATAACGTATTTAATTAA
TAAAACATTAAGTGTATACTTGGGATTGTTTTTATTGTAC
CCAGTAGGGATGATATTGCAATCTAATTATTTTTTTCAGG
CTACGAATAACAATAGGCCATTGGCTGTTTTTGTACTAAT
TGCTCGTGGTATGTCATTATGTCTTATTTATTTTTATAAT
GGACCAGCAGGCTATTTAACAAGTTATTATTATGTCATTT
GTGTGTCTGGTTCGTATTTTTTATCTGGCGTGCTATCGCT
TATATATATATATTATCAAAATAAGACTAATAAAGCTAAA
ATTCAATGGGCGGAAATTTTAGAATATATATGCACAGGTT
ATCATCTGTTTATTGCTAATATATTTGTTATTCTATACAG
AAATAGTAATATTATTATTCTTGGCACTCTTGCTTCGCCT
GTTGCAACGTCTCTGTACGCGACGGCAGAGAAAATTATTA
AATGTATTCAGTCTATAGCAACCCCGTTAAATCAATACTA
TTTCACGAGGTTGATAAAGCAACATGAATTGAAATTAGAA
CCATACAAAGTTGGAGAATATAAAAGCCTGCTATATGCAA
GCACAAATATTCAGCTAAAGTTCATGGTTTTCATTGTCCT
GAGTTTAGGGGGGGTGGGTACTATATTGGGATATAAGGTT
CAAAGTATCGCTGAAATTAGAAGCGCGTTCATCCCTTTAT
CAATAATGTCTTTTGCAATATTTATGGGGATATACAATTT
TATGTTTGGTTCGGTTGGATTGTCCATAAGAGGGTATAAA
AAAGAATTTTCTTATATAGTGGCCATTACGGGTGTTTCAA
CTATTATTTTATCATTATGCCTGAGTTATTTCTTTGCTGA
AATAGGCGCTGCAATTGCTTATGTATTTGCTGAGTTTATC
TTACTTATTCTCATACTTAGAATTTATAAAGTGAAACGAT
TATAATTCCCGGTAAGAACTGATAGTGCAAAATCATCATG
GCTATAGACTGAATAAATTGCGGGGAAAGAATGTATTACA
TCATATTTGTGATGGTTTTAGGTTTATGGATTATTGCATT
CAACTACGCTAGAGCAAACAAGCTAAGTAATTTGGTAATT
GTGCTTATTATTACTACTTTATTTATTATTAATAGGCAGA
ATCAAGACTATGAAGCGTATGTTGATATATTTAATGTCAA
TGAACTTTATGCCGAGATTGGTTATCGCTGGTTAATTTAT
GGTGTTAAGTATTTAGGCGGTACCCATGAAGTCATAATTG
GCTTGCTGGGTTTATTCCTTGGGACCACATTCCTACGATT
AATACAATACAGTAAGTATACAGCATTTGGTTTGCTGCTT
TACATGTTGTGTCCAATGCCTATTGACATTGTTCAAATCA
GGAATACATTTCTTTTTTTATTTGTCATAAATTCTCTTAT
CGAGTTAGAAAAAGAGCATAAATTTAACTCTCTTTGTTTT
GTCTTTATGGCACCTCTTTTCCATAGTTTAGGCATTGTTT
ATGTATTGGCGTGGGTAATAATCCAATTTCGTACCTGGAG
GGGTTATAATAAGTTAATGGTTTTTGGACTGGTGCTGTCT
TTTATTGTAGTGCCACTCTTGATTAAAATGTTAATTTTAT
TATTTAACACTAGGACGTTACATGCATATATAGCTGATGG
CATTAAGGTTCATTCTCTTATTATATGGGCAGGTCCTTAT
CTTTTTGATCTTTTTCTGTTATGTTACTTCAGAAAAAAAA
TAGTAATAACTGATTTACATACTAAACAATGGATTGATAT
AATATTCTCGCTAATGTTATTTCTTTCCGTATTTTCACCA
CTACTTTTATATATCGACGAAATAAATAGGATTTTTCGAA
ATGCGCTATTACTGAAGTATCTGGTAATGATGTCTATCGC
ACAGTATTTAAGTAGACCGACCAGATATATACTTTATTCA
TATTTACTGTTATTCACTTTTGCATTATCAATATATTATA
CACTCCAAATCGATTATGATTACATTGTATTTGGGTTACC
ATACTCATTATGATACTTATGAGGATATATGTTTTATTTT
ATCTGTGTAAATTATAATAACTCTGATTATACAGAAGCAT
TAATTAAGAGCGTTATTAATCAAGAAAAAGATAGCTTTGT
AATTGTTGTAGATAACTGTTCTAATGAGCAAGAGCTAAAA
AAGTTAAATGATATTGAGAGTAAATATAACGAGAAGGTCA
GATTAATTAAAAGTGATGTGAATTTGGGATATTTTGGCGG
ATTGAATCTTGGCCTAAAAGGACTACCAAGAAATCATCCA
ATAGTTGTTGGGAATAATGATTTGGTTTATGAGAGTAATT
TTACGCAAATAATATCACAGACTACATATCCGGACGATGC
TTTAGTTATTGCTCCAAATGTGATTACAAAGGATGGTTAT
CACCAGAACCCACACTGCCGTAAGCGAGTAAGTAAGTTTA
GGAAATTTTTGTATGATATATATTTTTCAAATTATATCGC
TGCTTTAGTTTTAACGTGGGGAAGCAGGTTTTTAAATTAT
TTGAAAGGTGGAAGAAATAATTCCTTTGATCAGGGTAGAG
GTTATATACATATGGGTATTGGAGCCTGTTATGTCCTTAT
GCCTTCCTTCTTCAAATATTTTGATGAATTAGATAACAAA
GTTTTTTTATATGGTGAGGAAGCATATTTGGCTGGCCAAT
TAATGGAAGTAAATGGGAAGATTTTCTATGAGCCTGATGC
TATTGTCCATCATGAAGAAAGTGCAACTTTGGCGAAAGTT
GCTTCTAAAACAAAATATGGCTATATGAAAAGCTCATATT
ACGACTATAAAAAATATCTGTAAAAGGTGAAAATATGTTT
AAGAATAAAACACTCGTTATCACTGGTGGCACTGGTTCTT
TTGGTAATGCCGTACTTAAGCGTTTTCTAGATACAGATAT
TACTGAAATACGAATATTTAGCAGGGATGAAAAAAAACAA
GATGATATGCGGAAAAAATATAATAACTCAAAATTAAAAT
TTTATATAGGTGATGTGCGAGACTATAATTCCGTTCTAAA
TGCAACGCGTGGTGCCGATTTTCTGTATCATGCAGCAGCC
CTTAAACAAGTTCCTTCATGTGAATTTCACCCTATGGAGG
CGGTTAAGACAAATGTTCTGGGTACGGAAAATGTTCTGGA
GGCTGCTATTGCGAATGGGATTAAACGCGTGGTGTGCTTG
AGTACCGATAAAGCCGTTTATCCTATCAATGCAATGGGCA
TATCTAAGGCAATGATGGAAAAAGTTATTGTTGCAAAATC
ACGTAATCTTGACAGTTCAAAAACAGTTATCTGTGGAACT
CGTTATGGAAATGTAATGGCTTCACGTGGATCGGTCATCC
CATTATTTGTTGATCTAATCAAAGCTGGTAAACCATTGAC
CATAACCGATCCCAATATGACTCGTTTCATGATGACGCTT
GAGGATGCTGTCGATCTGGTCCTTTATGCTTTCGAACATG
GAAATAATGGTGACATTTTCGTTCAGAAAGCACCTGCGGC
AACAATTCAAACATTAGCCATTGCACTTAAGGAATTGCTA
AATGCCCATGAGCATCCAATCAATATTATTGGAACTCGAC
ACGGGGAAAAACTTTACGAAGCGTTATTGAGCCGAGAGGA
AATGATTGCAGCGGAAGATATGGGTGATTATTATCGTGTT
CCACCAGATCTCCGCGATTTGAACTATGGAAAATATGTGG
AACATGGTGACCGTCGTATCTCGGAAGTGGAAGATTATAA
TTCTCATAATACTGAGAGATTAGATGTTGAGGGAATGAAA
GAATTACTGCTAAAACTTCCTTTTATCCGGGCACTTCGTT
CTGGTGAAGATTATGAGTTGGATTCATAATATGAAAATTT
TAGTTACTGGCGCTGCAGGGTTTATCGGTCGAAATTTGGT
TTTCCGCCTTAAGGAAGCTGGATATAACGAACTTATTGCG
ATAGATCGTAACTCTTCTTTGACGGATTTAGAGCAGGGAC
TTAAGCAGGCAGATTTCATTTTTCATCTTGCTGGAGTAAA
TCGTCCTGTGAAGGAGAGTGAGTTTGAAGAGGGGAATTGC
GATCTAACTCAACAGATTGTTGATATTCTGAAAAAAAATA
ATAAAGATACTCCTATCATGCTTAGTTCATCCATCCAAGC
TGAATGTGATAACGCTTATGGAAAGAGTAAAGCAGTTGCG
GAAAAAATCATTCAGCAGTATGGGGAAACGACAAACGCTA
AATATTATATTTATCGCTTGCCGAATGTATTCGGTAAGTG
GTGTCAACCAAATTATAACTCCTTTATAGCAACTTTCTGC
CATCGCATTGCAAATGATGAAGCTATTACAATTAATGATC
CTTCAGCAGTTGTAAATTTGGTGTATATAGATGACTTTTG
TTCTGACATATTAAAGCTATTAGAAGGAGCGAACGAAACT
GGTTACAGGACATTTGGTCCAATTTATTCTGTTACTGTTG
GTGAGGTGGCACAATTAATTTATCGATTTAAAGAAAGTCG
CCAAACATTAATCATCGAAGATGTAGGTAATGGATTCACT
CGAGCATTGTACTCAACATGGTTAAGTTACCTTTCTCCTA
AACAGTTTGCGTATACGGTTCCTTCTTATAGTGATGACAG
AGGGGTATTCTGTGAAGTATTGAAAACGAAAAACGCGGGC
CAGTTTTCGTTTTTTACTGCGCATCCAGGAATTACTCGGG
GGGGGCATTATCATCATTCCAAAAATGAGAAATTTATTAT
CATCCGAGGAAGTGCTTGTTTCAAATTTGAAAATATTGTC
ACGGGTGAACGATATGAACTTAATGTTTCATCAGATGATT
TTAAAATTGTTGAAACAGTTCCGGGATGGACGCATGACAT
TACTAATAATGGCTCGGATGAGCTAGTTGTTATGCTTTGG
GCAAATGAAATATTTAATCGTTCTGAACCAGATACTATAG
CGAGAGTTTTATCGTGAAAAAATTGAAAGTCATGTCGGTT
GTTGGGACTCGTCCAGAAATTATTCGACTCTCGCGTGTCC
TTGCAAAATTAGATGAATATTGTGATCACCTTATTGTTCA
TACTGGGCAAAATTACGATTATGAATTGAATGAGGTTTTT
TTCAAGGATTTGGGTGTTCGCAAACCTGATTATTTTCTTA
ATGCCGCAGGTAAAAATGCAGCAGAGACTATTGGACAAGT
TATCATTAAAGTTGATGAGGTCTTTGAACAGGAAAAACCA
GAAGCCATGTTAGTTCTTGGCGATACTAACTCCTGTATTT
CAGCAATACCAGCAAAGCGTCGAAGAATTCCGATTTTCCA
TATGGAGGCTGGGAATCGTTGTTTTGATCAACGCGTACCG
GAAGAAACTAACAGAAAAATAGTTGACCACACCGCTGATA
TCAATATGACATATAGTGATATCGCGCGTGAATATCTTTT
GGCTGAAGGTGTACCAGCCGATAGGATTATTAAAACCGGT
AGCCCAATGTTTGAAGTACTCACTCATTATATGTCGCAGA
TTGATGGTTCCGATGTACTTTCTCGCCTGAATTTAACACC
TGGGAATTTCTTTGTGGTAAGTGCCCACAGAGAAGAAAAT
GTTGATACCCCTAAACAGCTTGCGAAACTGGCAAATATAC
TTAATACCGTGGCTGAAAAATATGATATCCCGGTAGTTGT
TTCTACTCATCCGCGCACTCGTAACCGCATCAACGAAAAC
GGTATTCAATTTCATAAAAATATCTTGCTACTTAAGCCAT
TAGGATTTCACGATTACAACCATCTGCAAAAAAATGCACG
TGCTGTTTTATCGGATAGCGGGACTATTACAGAGGAGTCC
TCCATTATGAACTTCCCTGCGCTCAATATACGAGAAGCGC
ACGAACGCCCTGAAGGCTTCGAAGAAGGGGCTGTAATGAT
GGTTGGTCTTGAGTCTGCCCGTGTGTTACAGGCATTAGAA
ATTATTGCAACACAGCCTCGTGGAGAAGTACGCTTACTTC
GTCAGGTCAGTGACTATAGTATGCCAAATGTTTCAGATAA
AGTTGTGCGTATTATCCATTCATATACTGACTACGTTAAA
CGGGTTGTCTGGAAGCAATACTAATGAAACTTGCATTAAT
CATTGATGATTATTTGCCCCATAGCACACGAGTTGGGGCT
AAAATGTTTCATGAGTTAGGCCTGGAATTGCTGAGCAGAG
GCCATGATGTAACTGTAATTACGCCTGACAACACATTACA
GGCAATCTATTCTGTTAGCATGACTGATGGTATAAAGGTT
TGGCGTTTCAAAAGTGGACCTTTAAAGGATATAGGTAAGG
CTAAACGTGCCATAAATGAAACTCTTTTATCTTTTCGTGC
ATGGCACGCATTAAAGCATCTCATCCAACATGATACATTT
GATGGTATCGTTTATTATTCCCCCTCTATTTTTTGGGGAG
GCTTGGTTAAAAAAATTAAAGAGCGATGCCAGTGCCCAAG
CTATCTGGTCCTAAGGGATATGTTCCCACAGTGGGTCATT
GATGCTGGTATGATAAAAGCCGGCTCGCCAATTGAAAAAT
ATTTCAGGTATTTTGAAAAAAAATCATATCAACAGGCTGA
CTGGATTGGGTTAATGTCTGATAAGAATCTTGAGATATTT
CGTCAGGCCAATAAAGGTTATCCGTGTGAAGTTTTACGTA
ATTGGGCCTCAATGACTCCTGTGTCTGCCGGCGATGATTA
TCATTCACTTCGTCAAAAATACGCTCTAAAAGATAAAATC
ATTTTTTTCTATGGCGGTAATATTGGGCATGCTCAGGATA
TGGCAAACTTAATGCGCCTTGCGCGTAATATGATGCGTCA
TCATGATGCTCATTTCCTGTTTATAGGGCAGGGTGATGAA
GTTGACCTGATTAAATCTCTTTCTGCAGAATGGAATTTAA
CTAATTTCACTCATCTACCTTCAGTGAACCAGGAAGAGTT
TAAATTAATTTTATCTGAAGTTGATGTTGGCCTGTTCTCC
CTTTCGTCTCGCCATTCTTCACATAATTTCCCCGGAAAAT
TACTAGGGTATATGGTTCAATCAATCCCGATCCTTGGGAG
TGTGAATGGCGGTAATGATCTAATGGATGTAATTAATAAG
CACAGGGCTGGGTTCATTCATGTTAATGGTGAAGATGATA
AACTGTTTGAATCTGCACAATTGCTTCTTAGTGATTCAGT
TTTAAGAAAACAGTTAGGTCAGAACGCCAATGTGTTGTTA
AAGTCTCAATTTTCGGTTGAATCGGCGGCACATACTATCG
AAGTCCGACTGGAGGCAGGAGAATGCGTTTAGTTGATGAC
AATATTATGGATGAACTTTTTCGCACTGCAGCAAATTCTG
AACGTTTGCGCGCTCATTATTTATTGCACGCATCTCATCA
GGAGAAGGTTCAACGTTTACTTATTGCATTTGTACGCGAC
AGCTATGTTGAACCCCATTGGCATGAGTTACCGCATCAGT
GGGAAATGTTTGTCGTCATGCAAGGGCAATTAGAAGTTTG
TTTGTATGAGCAAAATGGTGAGCTCCAAAAAAAGTTTGTT
GTTGGAGACGGTACGGGAATAAGTGTCGTGGAATTTTCCC
CAGGAGATATACATAGTGTCAAATGCCTGTCACCAAAAGC
CCTTATGTTAGAGATAAAGGAGGGGCCATTTGACCCACTG
AAAGCTAAGGCTTTTTCTAAGTGGTTATAGGGCGATACAC
CACCGTTTATTCTTCTATCTTATTCTATACATGCTGGGTT
ACCATCTTAGCTTCTTCAAGCCGCGTAACCCCGCGTGACC
ACCCCTGACAGGAGTAAACAATGTCA
SEQ ID NO: 6
AF078736.1 Escherichia coli O111
O antigen gene cluster, partial sequence
GATCTGATGGCCGTAGGGCGCTACGTGCTTTCTGCTGATA
TCTGGGCTGAGTTGGAAAAAACTGCTCCAGGTGCCTGGGG
ACGTATTCAACTGACTGATGCTATTGCAGAGTTGGCTAAA
AAACAGTCTGTTGATGCCATGCTGATGACCGGCGACAGCT
ACGACTGCGGTAAGAAGATGGGCTATATGCAGGCATTCGT
TAAGTATGGGCTGCGCAACCTTAAAGAAGGGGCGAAGTTC
CGTAAGAGCATCAAGAAGCTACTGAGTGAGTAGAGATTTA
CACGTCTTTGTGACGATAAGCCAGAAAAAATAGCGGCAGT
TAACATCCAGGCTTCTATGCTTTAAGCAATGGAATGTTAC
TGCCGTTTTTTATGAAAAATGACCAATAATAACAAGTTAA
CCTACCAAGTTTAATCTGCTTTTTGTTGGATTTTTTCTTG
TTTCTGGTCGCATTTGGTAAGACAATTAGCGTGAGTTTTA
GAGAGTTTTGCGGGATCTCGCGGAACTGCTCACATCTTTG
GCATTTAGTTAGTGCACTGGTAGCTGTTAAGCCAGGGGCG
GTAGCTTGCCTAATTAATTTTTAACGTATACATTTATTCT
TGCCGCTTATAGCAAATAAAGTCAATCGGATTAAACTTCT
TTTCCATTAGGTAAAAGAGTGTTTGTAGTCGCTCAGGGAA
ATTGGTTTTGGTAGTAGTACTTTTCAAATTATCCATTTTC
CGATTTAGATGGCAGTTGATGTTACTATGCTGCATACATA
TCAATGTATATTATTTACTTTTAGAATGTGATATGAAAAA
AATAGTGATCATAGGCAATGTAGCGTCAATGATGTTAAGG
TTCAGGAAAGAATTAATCATGAATTTAGTGAGGCAAGGTG
ATAATGTATATTGTCTAGCAAATGATTTTTCCACTGAAGA
TCTTAAAGTACTTTCGTCATGGGGCGTTAAGGGGGTTAAA
TTCTCTCTTAACTCAAAGGGTATTAATCCTTTTAAGGATA
TAATTGCTGTTTATGAACTAAAAAAAATTCTTAAGGATAT
TTCCCCAGATATTGTATTTTCATATTTTGTAAAGCCAGTA
ATATTTGGAACTATTGCTTCAAAGTTGTCAAAAGTGCCAA
GGATTGTTGGAATGATTGAAGGTCTAGGTAATGCCTTCAC
TTATTATAAGGGAAAGCAGACCACAAAAACTAAAATGATA
AAGTGGATACAAATTCTTTTATATAAGTTAGCATTACCGA
TGCTTGATGATTTGATTCTATTAAATCATGATGATAAAAA
AGATTTAATCGATCAGTATAATATTAAAGCTAAGGTAACA
GTGTTAGGTGGGATTGGATTGGATCTTAATGAGTTTTCAT
ATAAAGAGCCACCGAAAGAGAAAATTACCTTTATTTTTAT
AGCAAGGTTATTAAGAGAGAAAGGGATATTTGAGTTTATT
GAAGCCGCAAAGTTCGTTAAGACAACTTATCCAAGTTCTG
AATTTGTAATTTTAGGAGGTTTTGAGAGTAATAATCCTTT
CTCATTACAAAAAAATGAAATTGAATCGCTAAGAAAAGAA
CATGATCTTATTTATCCTGGTCATGTGGAAAATGTTCAAG
ATTGGTTAGAGAAAAGTTCTGTTTTTGTTTTACCTACATC
ATATCGAGAAGGCGTACCAAGGGTGATCCAAGAAGCTATG
GCTATTGGTAGACCTGTAATAACAACTAATGTACCTGGGT
GTAGGGATATAATAAATGATGGGGTCAATGGCTTTTTGAT
ACCTCCATTTGAAATTAATTTACTGGCAGAAAAAATGAAA
TATTTTATTGAGAATAAAGATAAAGTACTCGAAATGGGGC
TTGCTGGAAGGAAGTTTGCAGAAAAAAACTTTGATGCTTT
TGAAAAAAATAATAGACTAGCATCAATAATAAAATCAAAT
AATGATTTTTGACTTGAGCAGAAATTATTTATATTTCAAT
CTGAAAAATAAAGGCTGTTATTATGAATAAAGTGGCATTA
ATTACTGGTATCACTGGGCAAGATGGCTCCTATTTGGCAG
AATTATTGTTAGAAAAAGGTTATGAAGTTCATGGTATTAA
ACGCCGTGCATCTTCATTTAATACTGAGCGAGTGGATCAC
ATCTATCAGGATTCACATTTAGCTAATCCTAAACTTTTTC
TACACTATGGCGATTTGACAGATACTTCCAATCTGACCCG
TATTTTAAAAGAAGTTCAACCAGATGAAGTTTACAATTTG
GGGGCGATGAGCCATGTAGCGGTATCATTTGAGTCACCAG
AATACACTGCTGATGTTGATGCGATAGGAACATTGCGTCT
TCTTGAAGCTATCAGGATATTGGGGCTGGAAAAAAAGACA
AAATTTTATCAGGCTTCAACTTCAGAGCTTTATGGTTTGG
TTCAAGAAATTCCACAAAAAGAGACTACGCCATTTTATCC
ACGTTCGCCTTATGCTGTTGCAAAATTATATGCCTATTGG
ATCACTGTTAATTATCGTGAGTCTTATGGTATGTTTGCCT
GCAATGGTATTCTCTTTAACCACGAATCACCTCGCCGTGG
CGAGACCTTTGTTACTCGTAAAATAACACGCGGGATAGCA
AATATTGCTCAAGGTCTTGATAAATGCTTATACTTGGGAA
ATATGGATTCTCTGCGTGATTGGGGACATGCTAAGGATTA
TGTCAAAATGCAATGGATGATGCTGCAGCAAGAAACTCCA
GAAGATTTTGTAATTGCTACAGGAATTCAATATTCTGTCC
GTGAGTTTGTCACAATGGCGGCAGAGCAAGTAGGCATAGA
GTTAGCATTTGAAGGTGAGGGAGTAAATGAAAAAGGTGTT
GTTGTTTCGGTCAATGGCACTGATGCTAAAGCTGTAAACC
CGGGCGATGTAATTATATCTGTAGATCCAAGGTATTTTAG
GCCTGCAGAAGTTGAAACCTTGCTTGGCGATCCTACTAAT
GCGCATAAAAAATTAGGATGGAGCCCTGAAATTACATTGC
GTGAAATGGTAAAAGAAATGGTTTCCAGCGATTTAGCAAT
AGCGAAAAAGAACGTCTTGCTGAAAGCTAATAACATTGCC
ACTAATATTCCGCAAGAATAAAAAAGATAATACATTAAAT
AATTAAAAATGGTGCTAGATTTATTAGTACCATTATTTTT
TTTTGGGTGACTAATGTTTATTACATCAGATAAATTTAGA
GAAATTATCAAGTTAGTTCCATTAGTATCAATTGATCTGC
TAATTGAAAACGAGAATGGTGAATATTTATTTGGTCTTAG
GAATAATCGACCGGCCAAAAATTATTTTTTTGTTCCAGGT
GGTAGGATTCGCAAAAATGAATCTATTAAAAATGCTTTTA
AAAGAATATCATCTATGGAATTAGGTAAAGAGTATGGTAT
TTCAGGAAGTGTTTTTAATGGTGTATGGGAACATTTCTAT
GATGATGGTTTTTTTTCTGAAGGCGAGGCAACACATTATA
TAGTGCTTTGTTACACACTGAAAGTTCTTAAAAGTGAATT
GAATCTCCCAGATGATCAACATCGTGAATACCTTTGGCTA
ACTAAACACCAAATAAATGCTAAACAAGATGTTCATAACT
ATTCAAAAAATTATTTTTTGTAATTTTTATTAAAAATTAA
TATGCGAGAGAATTGTATGTCTCAATGTCTTTACCCTGTA
ATTATTGCCGGAGGAACCGGAAGCCGTCTATGGCCGTTGT
CTCGAGTATTATACCCTAAACAATTTTTAAATTTAGTTGG
GGATTCTACAATGTTGCAAACAACAATTACGCGTTTGGAT
GGCATCGAATGCGAAAATCCAATTGTTATCTGCAATGAAG
ATCACCGATTTATTGTAGCAGAGCAATTACGACAGATTGG
TAAGCTAACCAAGAATATTATACTTGAGCCGAAAGGCCGT
AATACTGCACCTGCCATAGCTTTAGCTGCTTTTATCGCTC
AGAAGAATAATCCTAATGACGACCCTTTATTATTAGTACT
TGCGGCAGACCACTCTATAAATAATGAAAAAGCATTTCGA
GAGTCAATAATAAAAGCTATGCCGTATGCAACTTCTGGGA
AGTTAGTAACATTTGGAATTATTCCGGACACGGCAAATAC
TGGTTATGGATATATTAAGAGAAGTTCTTCAGCTGATCCT
AATAAAGAATTCCCAGCATATAATGTTGCGGAGTTTGTAG
AAAAACCAGATGTTAAAACAGCACAGGAATATATTTCGAG
TGGGAATTATTACTGGAATAGCGGAATGTTTTTATTTCGC
GCCAGTAAATATCTTGATGAACTACGGAAATTTAGACCAG
ATATTTATCATAGCTGTGAATGTGCAACCGCTACAGCAAA
TATAGATATGGACTTTGTCCGAATTAACGAGGCTGAGTTT
ATTAATTGTCCTGAAGAGTCTATCGATTATGCTGTGATGG
AAAAAACAAAAGACGCTGTAGTTCTTCCGATAGATATTGG
CTGGAATGACGTGGGTTCTTGGTCATCACTTTGGGATATA
AGCCAAAAGGATTGCCATGGTAATGTGTGCCATGGGGATG
TGCTCAATCATGATGGAGAAAATAGTTTTATTTACTCTGA
GTCAAGTCTGGTTGCGACAGTCGGAGTAAGTAATTTAGTA
ATTGTCCAAACCAAGGATGCTGTACTGGTTGCGGACCGTG
ATAAAGTCCAAAATGTTAAAAACATAGTTGACGATCTAAA
AAAGAGAAAACGTGCTGAATACTACATGCATCGTGCAGTT
TTTCGCCCTTGGGGTAAATTCGATGCAATAGACCAAGGCG
ATAGATATAGAGTAAAAAAAATAATAGTTAAACCAGGAGA
AGGGTTAGATTTAAGGATGCATCATCATAGGGCAGAGCAT
TGGATTGTTGTATCCGGTACTGCTAAAGTTTCACTAGGTA
GTGAAGTTAAACTATTAGTTTCTAATGAGTCTATATATAT
CCCTCAGGGAGCAAAATATAGTCTTGAGAATCCAGGCGTA
ATACCTTTGCATCTAATTGAAGTAAGTTCTGGTGATTACC
TTGAATCAGATGATATAGTGCGTTTTACTGACAGATATAA
CAGTAAACAATTCCTAAAGCGAGATTGATAAATATGAATA
AAATAACTTGCTTCAAAGCATATGATATACGTGGGCGTCT
TGGTGCTGAATTGAATGATGAAATAGCATATAGAATTGGT
CGCGCTTATGGTGAGTTTTTTAAACCTCAAACTGTAGTTG
TGGGAGGAGATGCTCGCTTAACAAGTGAGAGTTTAAAGAA
ATCACTCTCAAATGGGCTATGTGATGCAGGCGTAAATGTC
TTAGATCTTGGAATGTGTGGTACTGAAGAGATATATTTTT
CCACTTGGTATTTAGGAATTGATGGTGGAATCGAGGTAAC
TGCAAGCCATAATCCAATTGATTATAATGGAATGAAATTA
GTAACCAAAGGTGCTCGACCAATCAGCAGTGACACAGGTC
TCAAAGATATACAACAATTAGTAGAGAGTAATAATTTTGA
AGAGCTCAACCTAGAAAAAAAAGGGAATATTACCAAATAT
TCCACCCGAGATGCCTACATAAATCATTTGATGGGCTATG
CTAATCTGCAAAAAATAAAAAAAATCAAAATAGTTGTGAA
TTCTGGGAATGGTGCAGCTGGTCCTGTTATTGATGCTATT
GAGGAATGCTTTTTACGGAACAATATTCCGATTCAGTTTG
TAAAAATAAATAATACACCCGATGGTAATTTTCCACATGG
TATCCCTAATCCATTACTACCTGAGTGCAGAGAAGATACC
AGCAGTGCGGTTATAAGACATAGTGCTGATTTTGGTATTG
CATTTGATGGTGATTTTGATAGGTGTTTTTTCTTTGATGA
AAATGGACAATTTATTGAAGGATACTACATTGTTGGTTTA
TTAGCGGAAGTTTTTTTAGGGAAATATCCAAACGCAAAAA
TCATTCATGATCCTCGCCTTATATGGAATACTATTGATAT
CGTAGAAAGTCATGGTGGTATACCTATAATGACTAAAACC
GGTCATGCTTACATTAAGCAAAGAATGCGTGAAGAGGATG
CCGTATATGGCGGCGAAATGAGTGCGCATCATTATTTTAA
AGATTTTGCATACTGCGATAGTGGAATGATTCCTTGGATT
TTAATTTGTGAACTTTTGAGTCTGACAAATAAAAAATTAG
GTGAACTGGTTTGTGGTTGTATAAACGACTGGCCGGCAAG
TGGAGAAATAAACTGTACACTAGACAATCCGCAAAATGAA
ATAGATAAATTATTTAATCGTTACAAAGATAGTGCCTTAG
CTGTTGATTACACTGATGGATTAACTATGGAGTTCTCTGA
TTGGCGTTTTAATGTTAGATGCTCAAATACAGAACCTGTA
GTACGATTGAATGTAGAATCTAGGAATAATGCTATTCTTA
TGCAGGAAAAAACAGAAGAAATTCTGAATTTTATATCAAA
ATAAATTTGCACCTGAGTTCATAATGGGAACAAGAAATAT
ATGAAAGTACTTCTGACTGGCTCAACTGGCATGGTTGGTA
AGAATATATTAGAGCATGATAGTGCAAGTAAATATAATAT
ACTTACTCCAACCAGCTCTGATTTGAATTTATTAGATAAA
AATGAAATAGAAAAATTCATGCTTATCAACATGCCAGACT
GTATTATACATGCAGCGGGATTAGTTGGAGGCATTCATGC
AAATATAAGCAGGCCGTTTGATTTTCTGGAAAAAAATTTG
CAGATGGGTTTAAATTTAGTTTCCGTCGCAAAAAAACTAG
GTATCAAGAAAGTGCTTAACTTGGGTAGTTCATGCATGTA
CCCCAAAAACTTTGAAGAGGCTATTCCTGAGAAAGCTCTG
TTAACTGGTGAGCTAGAAGAAACTAATGAGGGATATGCTA
TTGCGAAAATTGCTGTAGCAAAAGCATGCGAATATATATC
AAGAGAAAACTCTAATTATTTTTATAAAACAATTATCCCA
TGTAATTTATATGGGAAATATGATAAATTTGATGATAACT
CGTCACATATGATTCCGGCAGTTATAAAAAAAATCCATCA
TGCGAAAATTAATAATGTCCCAGAGATCGAAATTTGGGGG
GATGGTAATTCGCGCCGTGAGTTTATGTATGCAGAAGATT
TAGCTGATCTTATTTTTTATGTTATTCCTAAAATAGAATT
CATGCCTAATATGGTAAATGCTGGTTTAGGTTACGATTAT
TCAATTAATGACTATTATAAGATAATTGCAGAAGAAATTG
GTTATACTGGGAGTTTTTCTCATGATTTAACAAAACCAAC
AGGAATGAAACGGAAGCTAGTAGATATTTCATTGCTTAAT
AAAATTGGTTGGTCAAGTCACTTTGAACTCAGAGATGGCA
TCAGAAAGACCTATAATTATTACTTGGAGAATCAAAATAA
ATGATTACATACCCACTTGCTAGTAATACTTGGGATGAAT
ATGAGTATGCAGCAATACAGTCAGTAATTGACTCAAAAAT
GTTTACCATGGGTAAAAAGGTTGAGTTATATGAGAAAAAT
TTTGCTGATTTGTTTGGTAGCAAATATGCCGTAATGGTTA
GCTCTGGTTCTACAGCTAATCTGTTAATGATTGCTGCCCT
TTTCTTCACTAATAAACCAAAACTTAAAAGAGGTGATGAA
ATAATAGTACCTGCAGTGTCATGGTCTACGACATATTACC
CTCTGCAACAGTATGGCTTAAAGGTGAAGTTTGTCGATAT
CAATAAAGAAACTTTAAATATTGATATCGATAGTTTGAAA
AATGCTATTTCAGATAAAACAAAAGCAATATTGACAGTAA
ATTTATTAGGTAATCCTAATGATTTTGCAAAAATAAATGA
GATAATAAATAATAGGGATATTATCTTACTAGAAGATAAC
TGTGAGTCGATGGGCGCGGTCTTTCAAAATAAGCAGGCAG
GCACATTCGGAGTTATGGGTACCTTTAGTTCTTTTTACTC
TCATCATATAGCTACAATGGAAGGGGGCTGCGTAGTTACT
GATGATGAAGAGCTGTATCATGTATTGTTGTGCCTTCGAG
CTCATGGTTGGACAAGAAATTTACCAAAAGAGAATATGGT
TACAGGCACTAAGAGTGATGATATTTTCGAAGAGTCGTTT
AAGTTTGTTTTACCAGGATACAATGTTCGCCCACTTGAAA
TGAGTGGTGCTATTGGGATAGAGCAACTTAAAAAGTTACC
AGGTTTTATATCCACCAGACGTTCCAATGCACAATATTTT
GTAGATAAATTTAAAGATCATCCATTCCTTGATATACAAA
AAGAAGTTGGTGAAAGTAGCTGGTTTGGTTTTTCCTTCGT
TATAAAGGAGGGAGCTGCTATTGAGAGGAAGAGTTTAGTA
AATAATCTGATCTCAGCAGGCATTGAATGCCGACCAATTG
TTACTGGGAATTTTCTCAAAAATGAACGTGTTTTGAGTTA
TTTTGATTACTCTGTACATGATACGGTAGCAAATGCCGAA
TATATAGATAAGAATGGTTTTTTTGTCGGAAACCACCAGA
TACCTTTGTTTAATGAAATAGATTATCTACGAAAAGTATT
AAAATAACTAACGAGGCACTCTATTTCGAATAGAGTGCCT
TTAAGATGGTATTAACAGTGAAAAAAATTTTAGCGTTTGG
CTATTCTAAAGTACTACCACCGGTTATTGAACAGTTTGTC
AATCCAATTTGCATCTTCATTATCACACCACTAATACTCA
ACCACCTGGGTAAGCAAAGCTATGGTAATTGGATTTTATT
AATTACTATTGTATCTTTTTCTCAGTTAATATGTGGAGGA
TGTTCCGCATGGATTGCAAAAATCATTGCAGAACAGAGAA
TTCTTAGTGATTTATCAAAAAAAAATGCTTTACGTCAAAT
TTCCTATAATTTTTCAATTGTTATTATCGCATTTGCGGTA
TTGATTTCTTTTCTTATATTAAGTATTTGTTTCTTCGATG
TTGCGAGGAATAATTCTTCATTCTTATTCGCGATTATTAT
TTGTGGTTTTTTTCAGGAAGTTGATAATTTATTTAGTGGT
GCGCTAAAAGGTTTTGAAAAATTTAATGTATCATGTTTTT
TTGAAGTAATTACAAGAGTGCTCTGGGCTTCTATAGTAAT
ATATGGCATTTACGGAAATGCACTCTTATATTTTACATGT
TTAGCCTTTACCATTAAAGGTATGCTAAAATATATTCTTG
TATGTCTGAATATTACCGGTTGTTTCATCAATCCTAATTT
TAATAGAGTTGGGATTGTTAATTTGTTAAATGAGTCAAAA
TGGATGTTTCTTCAATTAACTGGTGGCGTCTCACTTAGTT
TGTTTGATAGGCTCGTAATACCATTGATTTTATCTGTCAG
TAAACTGGCTTCTTATGTCCCTTGCCTTCAACTAGCTCAA
TTGATGTTCACTCTTTCTGCGTCTGCAAATCAAATATTAC
TACCAATGTTTGCTAGAATGAAAGCATCTAACACATTTCC
CTCTAATTGTTTTTTTAAAATTCTGCTTGTATCACTAATT
TCTGTTTTGCCTTGTCTTGCGTTATTCTTTTTTGGTCGTG
ATATATTATCAATATGGATAAACCCTACATTTGCAACTGA
AAATTATAAATTAATGCAAATTTTAGCTATAAGTTACATT
TTATTGTCAATGATGACATCTTTTCATTTCTTGTTATTAG
GAATTGGTAAATCTAAGCTTGTTGCAAATTTAAATCTGGT
TGCAGGGCTCGCACTTGCTGCTTCAACGTTAATCGCAGCT
CATTATGGCCTTTATGCAATATCTATGGTAAAAATAATAT
ATCCGGCTTTTCAATTTTATTACCTTTATGTAGCTTTTGT
CTATTTTAATAGAGCGAAAAATGTCTATTGATTTACTTTT
TTCAATTACTGAAATCGCAATTGTTTTTTCTTGCACTATT
TACATATTTACTCAATGTTTGTTAATGCGGAGGATCTATT
TAGATAAAAGTATTTTAATTCTTTTATGCTTGCTCTTTTT
TTTAGTAATCATTCAACTTCCTGAGCTTAATGTAAACGGT
TTGGTCGATTCTTTAAAGTTATCACTGCCTTTATTGATGG
TCTTTATCGCTTTTCAAAAACCGAAATTATGCTTGTGGGT
TATTATTGCATTGTTGTTTTTGAACTCTGCATTTAATTTT
TTATATTTAAAGACATTCGATAAGTTTAGCTCATTTCCTT
TTACTTTTTTTATATTGCTGTTTTACTTGTTTAGATTGGG
AATTGGTAATTTACCGGTTTATAAAAATAAAAAATTTTAC
GCGTTGATTTTTCTCTTTATATTAATAGACATAATGCAGT
CATTGTTAATAAATTATAGGGGGCAGATTTTATATTCCGT
AATTTGCATCCTGATACTTGTGTTTAAAGTTAATTTAAGA
AAAAAGATTCCATACTTTTTTTTAATGCTGCCAGTTTTAT
ATGTAATTATTATGGCTTATATTGGTTTTAATTATTTCAA
TAAAGGCGTAACTTTTTTTGAACCTACAGCAAGTAATATT
GAACGTACGGGGATGATATATTATTTGGTTTCACAGCTTG
GTGATTATATATTCCATGGTATGGGGACATTAAATTTCTT
AAATAACGGCGGACAATATAAGACGTTATATGGACTTCCA
TCATTAATTCCTAATGACCCTCATGATTTTTTATTACGGT
TCTTTATAAGTATTGGTGTGATAGGAGCATTGGTTTATCA
TTCTATATTTTTTGTTTTTTTTAGGAGAATATCTTTCTTA
TTATATGAGAGAAATGCTCCTTTCATTGTTGTAAGTTGTT
TGTTACTGTTACAAGTTGTGTTAATTTATACATTAAACCC
TTTTGATGCTTTTAATCGATTGATTTGCGGGCTTACAGTT
GGAGTTGTTTATGGATTTGCAAAAATTAGATAAGTATACC
TGTAATGGAAATTTAGACGCTCCACTTGTTTCAATAATCA
TTGCAACTTATAATTCTGAACTTGATATAGCTAAGTGTTT
GCAATCGGTAACTAATCAATCTTATAAGAATATTGAAATC
ATAATAATGGATGGAGGATCTTCTGATAAAACGCTTGATA
TTGCAAAATCGTTTAAAGACGACCGAATAAAAATAGTTTC
AGAGAAAGATCGTGGAATTTATGATGCCTGGAATAAAGCA
GTTGATTTATCCATTGGTGATTGGGTAGCATTTATTGGTT
CAGATGATGTTTACTATCATACAGATGCAATTGCTTCATT
GATGAAGGGGGTTATGGTATCTAATGGCGCCCCTGTGGTT
TATGGGAGGACAGCGCACGAAGGTCCCGATAGGAACATAT
CTGGATTTTCAGGCAGTGAATGGTACAACCTAACAGGATT
TAAGTTTAATTATTACAAATGTAATTTACCATTGCCCATT
ATGAGCGCAATATATTCTCGTGATTTCTTCAGAAACGAAC
GTTTTGATATTAAATTAAAAATTGTTGCTGACGCTGATTG
GTTTCTGAGATGTTTCATCAAATGGAGTAAAGAGAAGTCA
CCTTATTTTATTAATGACACGACCCCTATTGTTAGAATGG
GATATGGTGGGGTTTCGACTGATATTTCTTCTCAAGTTAA
AACTACGCTAGAAAGTTTCATTGTACGCAAAAAGAATAAT
ATATCCTGTTTAAACATACAGCTGATTCTTAGATATGCTA
AAATTCTGGTGATGGTAGCGATCAAAAATATTTTTGGCAA
TAATGTTTATAAATTAATGCATAACGGGTATCATTCCCTA
AAGAAAATCAAGAATAAAATATGAAGATTGTTTATATAAT
AACCGGGCTTACTTGTGGTGGAGCCGAACACCTTATGACG
CAGTTAGCAGACCAAATGTTTATACGCGGGCATGATGTTA
ATATTATTTGTCTAACTGGTATATCTGAGGTAAAGCCAAC
ACAAAATATTAATATTCATTATGTTAATATGGATAAAAAT
TTTAGAAGCTTTTTTAGAGCTTTATTTCAAGTAAAAAAAA
TAATTGTCGCCTTAAAGCCAGATATAATACATAGTCATAT
GTTTCATGCTAATATTTTTAGTCGTTTTATTAGGATGCTG
ATTCCAGCGGTGCCCCTGATATGTACCGCACACAACAAAA
ATGAAGGTGGCAATGCAAGGATGTTTTGTTATCGACTGAG
TGATTTTTTAGCTTCTATTACTACAAATGTAAGTAAAGAG
GCTGTTCAAGAGTTTATAGCAAGAAAGGCTACACCTAAAA
ATAAAATAGTAGAGATTCCGAATTTTATTAATACAAATAA
ATTTGATTTTGATATTAATGTCAGAAAGAAAACGCGAGAT
GCTTTTAATTTGAAAGACAGTACAGCAGTACTGCTCGCAG
TAGGAAGACTTGTTGAAGCAAAAGACTATCCGAACTTATT
AAATGCAATAAATCATTTGATTCTTTCAAAAACATCAAAT
TGTAATGATTTTATTTTGCTTATTGCTGGCGATGGCGCAT
TAAGAAATAAATTATTGGATTTGGTTTGTCAATTGAATCT
TGTGGATAAAGTTTTCTTCTTGGGGCAAAGAAGTGATATT
AAAGAATTAATGTGTGCTGCAGATCTTTTTGTTTTGAGTT
CTGAGTGGGAAGGTTTTGGTCTCGTTGTTGCAGAAGCTAT
GGCGTGTGAACGTCCCGTTGTTGCTACCGATTCTGGTGGA
GTTAAAGAAGTCGTTGGACCTCATAATGATGTTATCCCTG
TCAGTAATCATATTCTGTTGGCAGAGAAAATCGCTGAGAC
ACTTAAAATAGATGATAACGCAAGAAAAATAATAGGTATG
AAAAATAGAGAATATATTGTTTCCAATTTTTCAATTAAAA
CGATAGTGAGTGAGTGGGAGCGCTTATATTTTAAATATTC
CAAGCGTAATAATATAATTGATTGAAAATATAAGTTTGTA
CTCTGGATGCAATAGTTTCTCTATGCTGTTTTTTTACTGG
CTCCGTATTTTTACTTATAGCTGGATTTTGTTATATATCA
GTATTAATCTGTCTCAACTTCATCTAGACTACATTCAAGC
CGCGCATGCGTCGCGCGGTGACTACACCTGACAGGAGTAT
GTAATGTCCAAGCAACAGATCGGCGTCGTCGGTATGGCAG
TGATGGGGCGCAACCTGGCGCTCAACATCGAAAGCCGCGG
TTATACCGTCTCCATCTTCAACCGCTCCCGCGAGAAAACT
GAAGAAGTTGTTGCCGAGAACCCGGATAAGAAACTGGTTC
CTTATTACACGGTGAAAGAGTTCGTCGAGTCTCTTGAAAC
CCCACGTCGTATCCTGTTAATGGTAAAAGCAGGGGCGGGA
ACTGATGCTGCTATCGATTCCCTGAAGCCGTATCTGGATA
AAGGCGACATCATTATTGATGGTGGCAACACCTTCTTCCA
GGACACTATCCGTCGTAACCGTGAACTGTCCGCGGAAGGC
TTTAACTTCATCGGTACCGGCGTGTCCGGCGGTGAAGAGG
GCGCCCTGAAAGGCCCATCTATCATGCCAGGTGGCCAGAA
AGAAGCGTATGAGCTGGTTGCGCCTATCCTGACCAAGATT
GCTGCGGTTGCTGAAGATGGCGAACCATGTATAACTTACA
TCGGTGCTGACGGTGCGGGTCACTACGTGAAGATGGTGCA
CAACGGTATCGAATATGGCGATATGCAGCTGATTGCTGAA
GCCTATTCTCTGCTTAAAGGCGGCCTTAATCTGTCTAACG
AAGAGCTGGCAACCACTTTTACCGAGTGGAATGAAGGCGA
GCTAAGTAGCTACCTGATTGACATCACCAAAGACATCTTC
ACCAAAAAAGATGAAGAGGGTAAATACCTGGTTGATGTGA
TCCTGGACGAAGCTGCGAACAAAGGCACCGGTAAATGGAC
CAGCCAGAGCTCTCTGGATCTGGGTGAACCGCTGTCGCTG
ATCACCGAATCCGTATTCGCTCGCTACATCTCTTCTCTGA
AAGACCAGCGCATTGCGGCATCTAAAGTGCTGTCTGGTCC
GCAGGCTAAACTGGCTGGTGATAAAGCAGAGTTCGTTGAG
AAAGTCCGTCGCGCGCTGTACCTGGGTAAAATCGTCTCTT
ATGCCCAAGGCTTCTCTCAACTGCGTGCCGCGTCTGACGA
ATACAACTGGGATCTGAACTACGGCGAAATCGCGAAGATC
TTCCGCGCGGGCTGCATCATTCGTGCGCAGTTCCTGCAGA
AAATTACTGACGCGTATGCTGAAAACAAAGGCATTGCTAA
CCTGTTGCTGGCTCCGTACTTCAAAAATATCGCTGATGAA
TATCAGCAAGCGCTGCGTGATGTAGTGGCTTATGCTGTGC
AGAACGGTATTCCGGTACCGACCTTCTCTGCAGCGGTAGC
CTACTACGACAGCTACCGTTCTGCGGTACTGCCGGCTAAT
CTGATTCAGGCACAGCGTGATTACTTCGGTGCGCACACGT
ATAAACGCACTGATAAAGAAGGTGTGTTCCACACCG
SEQ ID NO: 7:
PRIMER FOR O103-wbtD+E-863F (forward)
5′-GTA AAT GGC GCG AAG CAA TA-3′
SEQ ID NO: 8:
PRIMER FOR O103-wbtD+E-1079R (reverse)
5′-TTG TTG GTT AAT GCA TGA CTC T-3′
SEQ ID NO: 9:
PRIMER FOR O121-vioA-210F (forward)
TTT GGG TGT TAA AGG CGA AG-3′
SEQ ID NO: 10:
PRIMER FOR O121-vioA-354R (reverse)
CGC CTC TTC AAT TCT TCT CG-3′
SEQ ID NO: 11:
026-Vir-Probe
5′-CAG ATA TTACTG AAA TAC G-3′-
SEQ ID NO: 12:
026 AVir Probe
5′-ACA GAT ATT GCT GAA-3′-
SEQ ID NO: 13:
O111 AVir Probe
5′-GTT ACA GGCACT AAG-3′-
SEQ ID NO: 14:
O111 Vir Probe
5′-GGT TAC A GG TAC TAA G-3′-
SEQ ID NO: 15:
O103 AVir Probe
5′-AT TAA CCT GCA TAT TAA A-3′-
SEQ ID NO: 16:
O103 Vir Probe
5′-AT TAA CCT GTA TAT TAA A-3′-
SEQ ID NO: 17:
O121 Vir Probe
5′-CG ATA TTG ATTCCA AAA CC-3′-
SEQ ID NO: 18:
O121 AVir Probe
5′-CG ATA TTG ATC CCA AAA CC-3′-
SEQ ID NO: 19:
16S RNA-F
5′-CCT CTT GCC ATC GGA TGT G-3′
SEQ ID NO: 20:
16S RNA-R
5′-GGC TGG TCA TCC TCT CAG ACC-3′
SEQ ID NO: 21:
16S rRNA Probe
5′-TAA CGG CTC ACC TAG GCG AC-3′
SEQ ID NO: 22:
Wzx O103-F
5′-TTG GAG CGT TAA CTG GAC CT-3′
SEQ ID NO: 23:
Wzx O103-R
5′-ATA TTC GCT ATA TCT TCT TGC GGC-3′
SEQ ID NO: 24:
Wzx O103-P
5′-AGG CTT ATC TGG CTG TTC TTA CTA CGG C--3′
SEQ ID NO: 25:
O103:H25-F:
5′-GAA ACA AAC GGT AAA TGG CG-3′
SEQ ID NO: 26:
O103:H25-R:
5′-TGT TGG TTA ATG CAT GAC TCT-3′
SEQ ID NO: 27:
(Probe):
5′-AGAATATTAACCTGCATATTA-3′
SEQ ID NO: 28:
(Probe):
5′-AGAACATTAACCTGCATATTA-3′

### REFERENCES

1. Boer, E. D., & Heuvelink, A. E. (2000). Methods for the detection and isolation of Shiga toxin-producing Escherichia coli. Journal of Applied Microbiology, 88(S1), 133S-143S. https://doi.org/10.1111/j.1365-2672.2000.tb05341.x
2. Etcheverría, A. I., & Padola, N. L. (2013). Shiga toxin-producing Escherichia coli. Virulence, 4(5), 366-372. https://doi.org/10.4161/viru.24642
3. Fratamico, P. M., Bagi, L. K., Cray, W. C., Narang, N., Yan, X., Medina, M., & Liu, Y. (2011). Detection by Multiplex Real-Time Polymerase Chain Reaction Assays and Isolation of Shiga Toxin—Producing Escherichia coli Serogroups O26, 45, O103, O111, O121, and O145 in Ground Beef. Foodborne Pathogens and Disease, 8(5), 601-607. https://doi.org/10.1089/fpd.2010.0773
4. Jana, M., Adriana, V., & Eva, K. (2020). Evaluation of DNA Extraction Methods for

Culture-Independent Real-Time PCR-Based Detection of Listeria monocytogenes in Cheese. Food Analytical Methods, 13(3), 667-677. https://doi.org/10.1007/s12161-019-01686-2
5. Li, F., Li, B., Dang, H., Kang, Q., Yang, L., Wang, Y., Aguilar, Z. P., Lai, W., & Xu, H.

(2017). Viable pathogens detection in fresh vegetables by quadruplex PCR. LWT - Food Science and Technology, 81, 306-313. https://doi.org/10.1016/j.wt.2017.03.064
6. Liu, Y., Singh, P., & Mustapha, A. (2018). High-resolution melt curve PCR assay for specific detection of E. coli O157:H7 in beef. Food Control, 86, 275-282. https://doi.org/10.1016/j.foodcont.2017.11.025
7. Lusk, T. S., Strain, E., & Kase, J. A. (2013). Comparison of six commercial DNA extraction kits for detection of Brucella neotomae in Mexican and Central American-style cheese and other milk products. Food Microbiology, 34(1), 100-105. https://doi.org/10.1016/j.fm.2012.11.007
8. Norman, K. N., Strockbine, N. A., & Bono, J. L. (2012). Association of Nucleotide

Polymorphisms within the 0-Antigen Gene Cluster of Escherichia coli O26, 45, O103, O111, O121, and O145 with Serogroups and Genetic Subtypes. Applied and Environmental Microbiology, 78(18), 6689-6703. https://doi.org/10.1128/AEM.01259-12
9. Singh, P., Liu, Y., Bosilevac, J. M., & Mustapha, A. (2019). Detection of Shiga toxin-producing Escherichia coli, stx1, stx2 and Salmonella by two high resolution melt curve multiplex real-time PCR. Food Control, 96, 251-259. https://doi.org/10.1016/j.foodcont.2018.09.024
10. Singh, P., & Mustapha, A. (2015). Multiplex real-time PCR assays for detection of eight

Shiga toxin-producing Escherichia coli in food samples by melting curve analysis. International Journal of Food Microbiology, 215, 101-108. https://doi.org/10.1016/j.ijfoodmicro.2015.08.022
11. Untergasser, A., Cutcutache, I., Koressaar, T., Ye, J., Faircloth, B. C., Remm, M., &

Rozen, S. G. (2012). Primer3—new capabilities and interfaces. Nucleic Acids Research, 40(15), e115—e115.
12. Wasilenko, J. L., Fratamico, P. M., Narang, N., Tillman, G. E., Ladely, S., Simmons, M., &

Cray, W. C. (2012). Influence of Primer Sequences and DNA Extraction Method on Detection of Non-O157 Shiga Toxin—Producing Escherichia coli in Ground Beef by Real-Time PCR Targeting the eae, stx, and Serogroup-Specific Genes. Journal of Food Protection, 75(11), 1939-1950. https://doi.org/10.4315/0362-028X.JFP-12-087

What is claimed is:

1. A method for detecting virulent Shiga toxin-producing E. coli (STEC) O26 strain in a biological sample comprising the steps of:
(i) enriching bacterial concentration of the biological sample to result in an enriched sample;
(ii) isolating DNA from said enriched biological sample; and
(iii) detecting virulent O26 in said isolated DNA sample using real-time PCR high-resolution melt curve assay or probe-based assays; wherein said real-time PCR comprises at least one primer pair, wherein said primer pair comprises SEQ ID NO: 1 and SEQ ID NO: 2.

2. The method of claim 1, wherein the probe-based assay comprises fluorescence resonance energy transfer (FRET) or dual-labeled probes or hydrolysis probes.

3. The method of claim 2, wherein the probe comprises SEQ ID NO: 11 or 12, or both.

4. The method of claim 2, wherein the probe-based assay comprises molecular beacon, Sunrise, or TaqMan® assay.

5. The method of claim 1, wherein virulent amplicons produced in step (iii) comprise a melting temperature differing by 0.2-4° C. from avirulent amplicons in said real-time PCR.

6. The method of claim 1, wherein said biological sample comprises a food or a beverage.

7. The method of claim 6, wherein said food or beverage comprises meat, produce, or juice.

8. The method of claim 1, wherein said biological sample comprises a clinical sample.

9. The method of claim 8, wherein said clinical sample comprises stool, urine, or blood.

10. The method of claim 1, wherein said real-time PCR comprises an internal amplification control SEQ ID NO: 19, SEQ ID NO: 20 and SEQ ID NO: 21.

11. A method for detecting virulent Shiga toxin-producing E. coli (STEC) O111 strain in a biological sample comprising the steps of:
(i) enriching bacterial concentration of the biological sample to result in an enriched sample;
(ii) isolating DNA from said enriched biological sample; and
(iii) detecting virulent O111 in said isolated DNA sample; wherein detection occurs via real-time PCR high-resolution melt assay or probe-based assay. Wherein said real-time PCR comprises at least one primer pair, wherein said primer pair comprises SEQ ID NO: 3 and SEQ ID NO: 4. and further wherein assay comprises one or more probes, wherein said probe comprises SEQ ID NO: 13 or 14, or both.

12. The method of claim 11, wherein the probe-based assay comprises fluorescence resonance energy transfer (FRET) or dual-labeled probes or hydrolysis probes.

13. The method of claim 11, wherein the probe-based assay comprises molecular beacon, Sunrise, or TaqMan® assay.

14. The method of claim 11, wherein virulent amplicons produced in step (iii) comprise a melting temperature differing by 0.2-4° C. from avirulent amplicons in said real-time PCR.

15. The method of claim 11, wherein said biological sample comprises a food or a beverage.

16. The method of claim 15, wherein said food or beverage comprises meat, produce, or juice.

17. The method of claim 11, wherein said biological sample comprises a clinical sample.

18. The method of claim 17, wherein said clinical sample comprises stool, urine, or blood.

19. The method of claim 11, wherein said real-time PCR comprises an internal amplification control SEQ ID NO: 19, SEQ ID NO: 20 and SEQ ID NO: 21.

20. A method for detecting virulent Shiga toxin-producing E. coli (STEC) O103 strain in a biological sample comprising the steps of:
(i) enriching bacterial concentration of the biological sample to result in an enriched sample;
(ii) isolating DNA from said enriched biological sample; and
(iii) detecting virulent O103 in said isolated DNA sample; wherein detection occurs via real-time PCR probe-based assays; wherein said real-time PCR comprises at least one primer pair; wherein said primer pair comprises SEQ ID NO: 7 and SEQ ID NO: 8; or SEQ ID NO:
22 and SEQ ID NO: 23; or SEQ ID NO: 25 and 26.

21. The method of claim 20, wherein the probe-based assay comprises fluorescence resonance energy transfer (FRET) or dual-labeled probes or hydrolysis probe.

22. The method of claim 20, wherein the probe-based assay comprises molecular beacon, Sunrise, or TaqMan® assay.

23. The method of claim 22, wherein the probe-based assay comprises one or more probes, and further wherein said probe comprises SEQ ID NO: 15 or 16, or both.

24. The method of claim 20, wherein virulent amplicons produced in step (iii) comprise a melting temperature differing by 0.2-4° C. from avirulent amplicons in said real-time PCR.

25. The method of claim 20, wherein said biological sample comprises a food or a beverage.

26. The method of claim 25, wherein said food or beverage comprises meat, produce, or juice.

27. The method of claim 20, wherein said biological sample comprises a clinical sample.

28. The method of claim 27, wherein said clinical sample comprises stool, urine, or blood.

29. The method of claim 21, wherein said real-time PCR comprises an internal amplification control SEQ ID NO: 19, SEQ ID NO: 20 and SEQ ID NO: 21.

30. A method for detecting virulent Shiga toxin-producing E. coli (STEC) O121 strain in a biological sample comprising the steps of:
(i) enriching bacterial concentration of the biological sample to result in an enriched sample;
(ii) isolating DNA from said enriched biological sample; and
(iii) detecting virulent O121 in said isolated DNA sample; wherein detection occurs via real-time PCR probe-based assays; wherein said real-time PCR comprises at least one primer pair; wherein said primer pair is SEQ ID NO: 9 or SEQ ID NO: 10.

31. The method of claim 30, wherein the probe-based assay comprises fluorescence resonance energy transfer (FRET) or dual-labeled probes or hydrolysis probes.

32. The method of claim 30, wherein the probe-based assay comprises molecular beacon, Sunrise, or TaqMan® assay.

33. The method of claim 30, wherein the real-time probe-based assay comprises one or more probes, wherein said probes comprise SEQ ID NO: 17 or 18, or both.

34. The method of claim 30, wherein virulent amplicons produced in step (iii) comprise a melting temperature differing by 0.2-4° C. from avirulent amplicons in said real-time PCR.

35. The method of claim 30, wherein said biological sample comprises a food or a beverage.

36. The method of claim 35, wherein said food or beverage comprises meat, produce, or juice.

37. The method of claim 30, wherein said biological sample comprises a clinical sample.

38. The method of claim 37, wherein said clinical sample comprises stool, urine, or blood.

39. The method of claim 30, wherein said real-time PCR comprises an internal amplification control SEQ ID NO: 19, SEQ ID NO: 20 and SEQ ID NO: 21.

40. An isolated nucleic acid sequence comprising 90% or more identity to SEQ ID NO: 1, 2, 3, 4, 7, 8, 9, 10, 11, 12, 13, 14, 15, 16, 17, 18, 22, 23, 24, 25, O26, 27, or 28.

41. The isolated nucleic acid sequence of claim 39, wherein the sequence comprises at least 95% identity to SEQ ID NO: 1, 2, 3, 4, 7, 8, 9, 10, 11, 12, 13, 14, 15, 16, 17, 18, 25, O26, 27, or 28.

42. The isolated nucleic acid sequence of claim 40, wherein the sequence comprises SEQ ID NO: 1, 2, 3, 4, 7, 8, 9, 10, 11, 12, 13, 14, 15, 16, 17, 18, 25, O26, 27, or 28.

43. A kit comprising a primer pair, wherein the primer pair comprises SEQ ID NO: 1 and SEQ ID NO: 2.

44. The kit of claim 43, wherein said kit further comprises additional reagents for amplification.

45. The kit of claim 43, wherein the kit further comprises SEQ ID NO: 11 and 12.

46. A kit comprising a primer pair, wherein the primer pair comprises SEQ ID NO: 3 and SEQ ID NO: 4 and wherein the kit further comprises SEQ ID NO: 13 and 14.

47. The kit of claim 46, wherein said kit further comprises additional reagents for amplification.

48. A kit comprising a primer pair, wherein the primer pair comprises SEQ ID NO: 7 and SEQ ID NO: 8.

49. The kit of claim 48, wherein said kit further comprises additional reagents for amplification.

50. The kit of claim 48, wherein the kit further comprises SEQ ID NO: 15 and 16.

51. A kit comprising a primer pair, wherein the primer pair comprises SEQ ID NO: 9 and SEQ ID NO: 10.

52. The kit of claim 51, wherein said kit further comprises additional reagents for amplification.

53. The kit of claim 51, wherein the kit further comprises SEQ ID NO: 17 or 18.


SEQUENCE LISTING (28 sequences)

SEQ ID NO: 1 - DNA, 20 bp, synthetic construct
  misc_feature 1..20: note = Description of sequence: Primer
  misc_feature 1..20: note = Name: O26 fnl1-32F (forward primer)
  source 1..20: mol_type = other DNA
gtggcactggttcttttggt

SEQ ID NO: 2 - DNA, 20 bp, synthetic construct
  misc_feature 1..20: note = Description of sequence: Primer
  misc_feature 1..20: note = Name: O26 fnl1-219R (reverse primer)
  source 1..20: mol_type = other DNA
accacgcgttgcatttagaa

SEQ ID NO: 3 - DNA, 20 bp, synthetic construct
  misc_feature 1..20: note = Description of sequence: Primer
  misc_feature 1..20: note = Name: O111 wbdK-634F (forward primer)
  source 1..20: mol_type = other DNA
cttcgagctcatggttggac

SEQ ID NO: 4 - DNA, 19 bp, synthetic construct
  misc_feature 1..19: note = Description of sequence: Primer
  misc_feature 1..19: note = Name: O111 wbdK-717R (reverse primer)
  source 1..19: mol_type = other DNA
cgactcttcgaaaatatca

SEQ ID NO: 5 - DNA, 11706 bp, Escherichia coli
  misc_feature 1..11706: note = AY763106.1
  misc_feature 1..11706: note = serotype O26
  misc_feature 1..11706: note = H11 O-antigen operon partial sequence
  source 1..11706: mol_type = genomic DNA
gcattaatacccctattaatcaacctaagagccgcttatttcacagcatgctctgaagtaatatggaataataaagtgaagatacttgttactggtggcgcaggatttattggttctgctgtagttcgtcacattataaataatacgcaggatagtgttgttaatgtcgataaattaacgtacgccggaaacctggaatcacttgctgatgtttctgattctgaacgctatgtttttgaacatgcggatatttgcgatgcagctgcaatggcgcggatttttgctcagcatcagccggatgcagtgatgcacctggctgctgaaagccatgttgaccgttcaattaccggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatcctgacgaagtaaataatacagaagaattacccttatttactgagacgacagcttacgcgccaagcagtccttattccgcatccaaagcatctagcgatcatttagtccgcgcgtggaaacgtacctatggtttaccgaccattgtgactaactgttcgaataactacggcccttatcactttccggaaaaattgattccgctggtaattcttaatgctctggaaggtaaggcattacctatttatggcaaaggggatcaaattcgcgattggctatatgttgaagatcatgcgcgtgcgttatataccgttgtcaccgaaggtaaagcgggtgaaacttataacattggcggacacaacgaaaagaaaaacatcgatgttgtgctgactatttgtgatttgttggatgagattgtaccgaaagagcaatcttatcgtgagcaaattacttatgttgccgatcgtccgggacacgatcgccgttatgcgattgatgctgagaagattagccgcgaattgggctggaaaccgcaggaaacgtttgagagcgggattcggaagacagtggaatggtacctgtccaatacaaaatgggtcgaaaatgtgaaaagtggtgcctatcagtcatggattgcacagaactatgagggccgtcagtaatgaatatcctccttttcggcaaaacagggcaggtaggttgggaactacagcgtgctctggcacctctgggtaatttgattgctcttgatgttcactctactgattattgcggtgattttagtaatcctgaaggtgtagctgaaaccgtaagaagcattcggcctgatattattgtcaacgcagccgctcacaccgcagtagacaaagcagaatcagaaccggagtttgcacaattacttaacgcaacaagtgtcgaagcgattgcgaaagcagccaatgaagtcggcgcctggcttattcattactcgactgattacgtcttccctggaaatggcgatatgccatggcgggagacggatgcaaccgcaccactaaatgtttacggtgaaaccaagttagccggagaaaaagcgttacaggaatattgcgcaaagcatcttattttccggaccagctgggtctatgcaggaaaaggaaataacttcgccaaaacgatgttacgtctggcaaaagagcgtgaagaattagcggttattaatgatcagtttggtgcgccaacaggtgctgaactgctggctgattgtacagcacatgccattcgtgtcgcactgaataaaccagaagtcgcaggcttgtaccatctggtagccagtggtaccacaacctggcacgattatgctgcgctggtttttgaagaggcacgcaaagcaggtattccccttgcactcaacaagctcaacgcagtaccaacaacagcctatcctacaccagctcgtcgtccgcataactctcgccttaatacagaaaaatttcagcagaaatttgcgcttgttttgcctgattggcaggttggcgtgaaacgaatgctcaacgaattatttacgactacagcaatttaatagtttttgcatcttgttcgtgatggtggagcaagatgaattaaaaggaatgatgaaatgaaaacgcgtaaaggtattattttagctggtggttcgggtactcgtctttatcctgtaactatggctgtcagtaaacagttgttaccgatttatgataaaccgatgatctattacccgttgtctacactgatgttagcgggtcttcgcgatattctgattattagtacgccacaggatactcctcgttttcaacaactgctgggtgacgggagccagtgggggctaaatcttcagtacaaagtgcaaccgagtccagatggtcttgcgcaggcatttatcatcggtgaagagtttatcggtggtgatgattgtgctttggttctaggtgataatatcttttacggtcacgatctgccgaagttaatggatgtcgctgttaacaaagaaagtggtgcaacggtatttgcctatcacgttaatgatcctgaacgctacggtgtcgttgagtttgataaaaacggtacggcgatcagcctggaagaaaaaccgctacaaccaaaaagtaattatgcggtaaccgggctttatttttatgataactacgttgtcgaaatggcgaaaaatcttaagccttctgcccgcggtgaactggaaattaccgatattaaccgtatttatatggaacaggggcgtttatccgttgccatgatgggacgtggttatgcatggctggacacggggacacatcaaagtcttattgaggcaagcaattttatcgcaacaatagaagaacgtcaggggctgaaagtttcctgcccggaagaaattgcttaccgtaaagggtttatcgatgctgagcaggtgaaagtattagctgaaccgttgaagaaaaatgcttatggtcagtatctgctgaaaatgattaaaggttattaataaaatgaacgtaattaaaacagaaattccagatgtactgatttttgaaccgaaagtttttggtgatgagcgtggtttctttatggaaagctttaatcagaaagtttttgaagaagctgtagggcggaaggttgaatttgttcaggataatcattctaaatcatctaagggtgtgttacgcggactgcactatcagctggaaccctatgctcaaggtaaattagttcgttgtgtggtcggtgaagtatttgatgtagcagttgatattcgtaaatcgtcacctacatttgggaaatgggttggggtgaatttgtctgctgagaataagcgtcagttgtggatccctgaggggtttgcgcatggttttttggtgttaagcgagatagcagagtttgtttataagacgacaaattattatcatcctgaatcagaaggttgcataaaatgggatgattcttttttgatgattgattggccaaataaaccgatagcaatctcagaaaaagataaaaaaggctcatcaattttgaggctaaatgactattgatgttgaaaaaaaaacttcaaaaaataaaggaatatcattcagtattggagttggcaataattcagggtgcgaatgccatatttcctgtgttggtattcccattttttcttattaccttaggggaaaacatcttttcaagtattgctgttggtgaagtactagcactatatgtgcttatattttcgctatacagttttgatattataagtgtgcagaaggtaatttcaagtgtgacaaaagatgaaatatttaaagtttacattctgacactaatctgtaggttgtgtttatttgttatttcaggaatatgtcttttatttataacgtatttaattaataaaacattaagtgtatacttgggattgtttttattgtacccagtagggatgatattgcaatctaattatttttttcaggctacgaataacaataggccattggctgtttttgtactaattgctcgtggtatgtcattatgtcttatttatttttataatggaccagcaggctatttaacaagttattattatgtcatttgtgtgtctggttcgtattttttatctggcgtgctatcgcttatatatatatattatcaaaataagactaataaagctaaaattcaatgggcggaaattttagaatatatatgcacaggttatcatctgtttattgctaatatatttgttattctatacagaaatagtaatattattattcttggcactcttgcttcgcctgttgcaacgtctctgtacgcgacggcagagaaaattattaaatgtattcagtctatagcaaccccgttaaatcaatactatttcacgaggttgataaagcaacatgaattgaaattagaaccatacaaagttggagaatataaaagcctgctatatgcaagcacaaatattcagctaaagttcatggttttcattgtcctgagtttagggggggtgggtactatattgggatataaggttcaaagtatcgctgaaattagaagcgcgttcatccctttatcaataatgtcttttgcaatatttatggggatatacaattttatgtttggttcggttggattgtccataagagggtataaaaaagaattttcttatatagtggccattacgggtgtttcaactattattttatcattatgcctgagttatttctttgctgaaataggcgctgcaattgcttatgtatttgctgagtttatcttacttattctcatacttagaatttataaagtgaaacgattataattcccggtaagaactgatagtgcaaaatcatcatggctatagactgaataaattgcggggaaagaatgtattacatcatatttgtgatggttttaggtttatggattattgcattcaactacgctagagcaaacaagctaagtaatttggtaattgtgcttattattactactttatttattattaataggcagaatcaagactatgaagcgtatgttgatatatttaatgtcaatgaactttatgccgagattggttatcgctggttaatttatggtgttaagtatttaggcggtacccatgaagtcataattggcttgctgggtttattccttgggaccacattcctacgattaatacaatacagtaagtatacagcatttggtttgctgctttacatgttgtgtccaatgcctattgacattgttcaaatcaggaatacatttctttttttatttgtcataaattctcttatcgagttagaaaaagagcataaatttaactctctttgttttgtctttatggcacctcttttccatagtttaggcattgtttatgtattggcgtgggtaataatccaatttcgtacctggaggggttataataagttaatggtttttggactggtgctgtcttttattgtagtgccactcttgattaaaatgttaattttattatttaacactaggacgttacatgcatatatagctgatggcattaaggttcattctcttattatatgggcaggtccttatctttttgatctttttctgttatgttacttcagaaaaaaaatagtaataactgatttacatactaaacaatggattgatataatattctcgctaatgttatttctttccgtattttcaccactacttttatatatcgacgaaataaataggatttttcgaaatgcgctattactgaagtatctggtaatgatgtctatcgcacagtatttaagtagaccgaccagatatatactttattcatatttactgttattcacttttgcattatcaatatattatacactccaaatcgattatgattacattgtatttgggttaccatactcattatgatacttatgaggatatatgttttattttatctgtgtaaattataataactctgattatacagaagcattaattaagagcgttattaatcaagaaaaagatagctttgtaattgttgtagataactgttctaatgagcaagagctaaaaaagttaaatgatattgagagtaaatataacgagaaggtcagattaattaaaagtgatgtgaatttgggatattttggcggattgaatcttggcctaaaaggactaccaagaaatcatccaatagttgttgggaataatgatttggtttatgagagtaattttacgcaaataatatcacagactacatatccggacgatgctttagttattgctccaaatgtgattacaaaggatggttatcaccagaacccacactgccgtaagcgagtaagtaagtttaggaaatttttgtatgatatatatttttcaaattatatcgctgctttagttttaacgtggggaagcaggtttttaaattatttgaaaggtggaagaaataattcctttgatcagggtagaggttatatacatatgggtattggagcctgttatgtccttatgccttccttcttcaaatattttgatgaattagataacaaagtttttttatatggtgaggaagcatatttggctggccaattaatggaagtaaatgggaagattttctatgagcctgatgctattgtccatcatgaagaaagtgcaactttggcgaaagttgcttctaaaacaaaatatggctatatgaaaagctcatattacgactataaaaaatatctgtaaaaggtgaaaatatgtttaagaataaaacactcgttatcactggtggcactggttcttttggtaatgccgtacttaagcgttttctagatacagatattactgaaatacgaatatttagcagggatgaaaaaaaacaagatgatatgcggaaaaaatataataactcaaaattaaaattttatataggtgatgtgcgagactataattccgttctaaatgcaacgcgtggtgccgattttctgtatcatgcagcagcccttaaacaagttccttcatgtgaatttcaccctatggaggcggttaagacaaatgttctgggtacggaaaatgttctggaggctgctattgcgaatgggattaaacgcgtggtgtgcttgagtaccgataaagccgtttatcctatcaatgcaatgggcatatctaaggcaatgatggaaaaagttattgttgcaaaatcacgtaatcttgacagttcaaaaacagttatctgtggaactcgttatggaaatgtaatggcttcacgtggatcggtcatcccattatttgttgatctaatcaaagctggtaaaccattgaccataaccgatcccaatatgactcgtttcatgatgacgcttgaggatgctgtcgatctggtcctttatgctttcgaacatggaaataatggtgacattttcgttcagaaagcacctgcggcaacaattcaaacattagccattgcacttaaggaattgctaaatgcccatgagcatccaatcaatattattggaactcgacacggggaaaaactttacgaagcgttattgagccgagaggaaatgattgcagcggaagatatgggtgattattatcgtgttccaccagatctccgcgatttgaactatggaaaatatgtggaacatggtgaccgtcgtatctcggaagtggaagattataattctcataatactgagagattagatgttgagggaatgaaagaattactgctaaaacttccttttatccgggcacttcgttctggtgaagattatgagttggattcataatatgaaaattttagttactggcgctgcagggtttatcggtcgaaatttggttttccgccttaaggaagctggatataacgaacttattgcgatagatcgtaactcttctttgacggatttagagcagggacttaagcaggcagatttcatttttcatcttgctggagtaaatcgtcctgtgaaggagagtgagtttgaagaggggaattgcgatctaactcaacagattgttgatattctgaaaaaaaataataaagatactcctatcatgcttagttcatccatccaagctgaatgtgataacgcttatggaaagagtaaagcagttgcggaaaaaatcattcagcagtatggggaaacgacaaacgctaaatattatatttatcgcttgccgaatgtattcggtaagtggtgtcaaccaaattataactcctttatagcaactttctgccatcgcattgcaaatgatgaagctattacaattaatgatccttcagcagttgtaaatttggtgtatatagatgacttttgttctgacatattaaagctattagaaggagcgaacgaaactggttacaggacatttggtccaatttattctgttactgttggtgaggtggcacaattaatttatcgatttaaagaaagtcgccaaacattaatcatcgaagatgtaggtaatggattcactcgagcattgtactcaacatggttaagttacctttctcctaaacagtttgcgtatacggttccttcttatagtgatgacagaggggtattctgtgaagtattgaaaacgaaaaacgcgggccagttttcgttttttactgcgcatccaggaattactcggggggggcattatcatcattccaaaaatgagaaatttattatcatccgaggaagtgcttgtttcaaatttgaaaatattgtcacgggtgaacgatatgaacttaatgtttcatcagatgattttaaaattgttgaaacagttccgggatggacgcatgacattactaataatggctcggatgagctagttgttatgctttgggcaaatgaaatatttaatcgttctgaaccagatactatagcgagagttttatcgtgaaaaaattgaaagtcatgtcggttgttgggactcgtccagaaattattcgactctcgcgtgtccttgcaaaattagatgaatattgtgatcaccttattgttcatactgggcaaaattacgattatgaattgaatgaggtttttttcaaggatttgggtgttcgcaaacctgattattttcttaatgccgcaggtaaaaatgcagcagagactattggacaagttatcattaaagttgatgaggtctttgaacaggaaaaaccagaagccatgttagttcttggcgatactaactcctgtatttcagcaataccagcaaagcgtcgaagaattccgattttccatatggaggctgggaatcgttgttttgatcaacgcgtaccggaagaaactaacagaaaaatagttgaccacaccgctgatatcaatatgacatatagtgatatcgcgcgtgaatatcttttggctgaaggtgtaccagccgataggattattaaaaccggtagcccaatgtttgaagtactcactcattatatgtcgcagattgatggttccgatgtactttctcgcctgaatttaacacctgggaatttctttgtggtaagtgcccacagagaagaaaatgttgatacccctaaacagcttgcgaaactggcaaatatacttaataccgtggctgaaaaatatgatatcccggtagttgtttctactcatccgcgcactcgtaaccgcatcaacgaaaacggtattcaatttcataaaaatatcttgctacttaagccattaggatttcacgattacaaccatctgcaaaaaaatgcacgtgctgttttatcggatagcgggactattacagaggagtcctccattatgaacttccctgcgctcaatatacgagaagcgcacgaacgccctgaaggcttcgaagaaggggctgtaatgatggttggtcttgagtctgcccgtgtgttacaggcattagaaattattgcaacacagcctcgtggagaagtacgcttacttcgtcaggtcagtgactatagtatgccaaatgtttcagataaagttgtgcgtattatccattcatatactgactacgttaaacgggttgtctggaagcaatactaatgaaacttgcattaatcattgatgattatttgccccatagcacacgagttggggctaaaatgtttcatgagttaggcctggaattgctgagcagaggccatgatgtaactgtaattacgcctgacaacacattacaggcaatctattctgttagcatgactgatggtataaaggtttggcgtttcaaaagtggacctttaaaggatataggtaaggctaaacgtgccataaatgaaactcttttatcttttcgtgcatggcacgcattaaagcatctcatccaacatgatacatttgatggtatcgtttattattccccctctattttttggggaggcttggttaaaaaaattaaagagcgatgccagtgcccaagctatctggtcctaagggatatgttcccacagtgggtcattgatgctggtatgataaaagccggctcgccaattgaaaaatatttcaggtattttgaaaaaaaatcatatcaacaggctgactggattgggttaatgtctgataagaatcttgagatatttcgtcaggccaataaaggttatccgtgtgaagttttacgtaattgggcctcaatgactcctgtgtctgccggcgatgattatcattcacttcgtcaaaaatacgctctaaaagataaaatcatttttttctatggcggtaatattgggcatgctcaggatatggcaaacttaatgcgccttgcgcgtaatatgatgcgtcatcatgatgctcatttcctgtttatagggcagggtgatgaagttgacctgattaaatctctttctgcagaatggaatttaactaatttcactcatctaccttcagtgaaccaggaagagtttaaattaattttatctgaagttgatgttggcctgttctccctttcgtctcgccattcttcacataatttccccggaaaattactagggtatatggttcaatcaatcccgatccttgggagtgtgaatggcggtaatgatctaatggatgtaattaataagcacagggctgggttcattcatgttaatggtgaagatgataaactgtttgaatctgcacaattgcttcttagtgattcagttttaagaaaacagttaggtcagaacgccaatgtgttgttaaagtctcaattttcggttgaatcggcggcacatactatcgaagtccgactggaggcaggagaatgcgtttagttgatgacaatattatggatgaactttttcgcactgcagcaaattctgaacgtttgcgcgctcattatttattgcacgcatctcatcaggagaaggttcaacgtttacttattgcatttgtacgcgacagctatgttgaaccccattggcatgagttaccgcatcagtgggaaatgtttgtcgtcatgcaagggcaattagaagtttgtttgtatgagcaaaatggtgagctccaaaaaaagtttgttgttggagacggtacgggaataagtgtcgtggaattttccccaggagatatacatagtgtcaaatgcctgtcaccaaaagcccttatgttagagataaaggaggggccatttgacccactgaaagctaaggctttttctaagtggttatagggcgatacaccaccgtttattcttctatcttattctatacatgctgggttaccatcttagcttcttcaagccgcgtaaccccgcgtgaccacccctgacaggagtaaacaatgtca

SEQ ID NO: 6 - DNA, 14516 bp, Escherichia coli
  misc_feature 1..14516: note = AF078736.1
  misc_feature 1..14516: note = O111 O antigen gene cluster partial sequence
  source 1..14516: mol_type = genomic DNA
gatctgatggccgtagggcgctacgtgctttctgctgatatctgggctgagttggaaaaaactgctccaggtgcctggggacgtattcaactgactgatgctattgcagagttggctaaaaaacagtctgttgatgccatgctgatgaccggcgacagctacgactgcggtaagaagatgggctatatgcaggcattcgttaagtatgggctgcgcaaccttaaagaaggggcgaagttccgtaagagcatcaagaagctactgagtgagtagagatttacacgtctttgtgacgataagccagaaaaaatagcggcagttaacatccaggcttctatgctttaagcaatggaatgttactgccgttttttatgaaaaatgaccaataataacaagttaacctaccaagtttaatctgctttttgttggattttttcttgtttctggtcgcatttggtaagacaattagcgtgagttttagagagttttgcgggatctcgcggaactgctcacatctttggcatttagttagtgcactggtagctgttaagccaggggcggtagcttgcctaattaatttttaacgtatacatttattcttgccgcttatagcaaataaagtcaatcggattaaacttcttttccattaggtaaaagagtgtttgtagtcgctcagggaaattggttttggtagtagtacttttcaaattatccattttccgatttagatggcagttgatgttactatgctgcatacatatcaatgtatattatttacttttagaatgtgatatgaaaaaaatagtgatcataggcaatgtagcgtcaatgatgttaaggttcaggaaagaattaatcatgaatttagtgaggcaaggtgataatgtatattgtctagcaaatgatttttccactgaagatcttaaagtactttcgtcatggggcgttaagggggttaaattctctcttaactcaaagggtattaatccttttaaggatataattgctgtttatgaactaaaaaaaattcttaaggatatttccccagatattgtattttcatattttgtaaagccagtaatatttggaactattgcttcaaagttgtcaaaagtgccaaggattgttggaatgattgaaggtctaggtaatgccttcacttattataagggaaagcagaccacaaaaactaaaatgataaagtggatacaaattcttttatataagttagcattaccgatgcttgatgatttgattctattaaatcatgatgataaaaaagatttaatcgatcagtataatattaaagctaaggtaacagtgttaggtgggattggattggatcttaatgagttttcatataaagagccaccgaaagagaaaattacctttatttttatagcaaggttattaagagagaaagggatatttgagtttattgaagccgcaaagttcgttaagacaacttatccaagttctgaatttgtaattttaggaggttttgagagtaataatcctttctcattacaaaaaaatgaaattgaatcgctaagaaaagaacatgatcttatttatcctggtcatgtggaaaatgttcaagattggttagagaaaagttctgtttttgttttacctacatcatatcgagaaggcgtaccaagggtgatccaagaagctatggctattggtagacctgtaataacaactaatgtacctgggtgtagggatataataaatgatggggtcaatggctttttgatacctccatttgaaattaatttactggcagaaaaaatgaaatattttattgagaataaagataaagtactcgaaatggggcttgctggaaggaagtttgcagaaaaaaactttgatgcttttgaaaaaaataatagactagcatcaataataaaatcaaataatgatttttgacttgagcagaaattatttatatttcaatctgaaaaataaaggctgttattatgaataaagtggcattaattactggtatcactgggcaagatggctcctatttggcagaattattgttagaaaaaggttatgaagttcatggtattaaacgccgtgcatcttcatttaatactgagcgagtggatcacatctatcaggattcacatttagctaatcctaaactttttctacactatggcgatttgacagatacttccaatctgacccgtattttaaaagaagttcaaccagatgaagtttacaatttgggggcgatgagccatgtagcggtatcatttgagtcaccagaatacactgctgatgttgatgcgataggaacattgcgtcttcttgaagctatcaggatattggggctggaaaaaaagacaaaattttatcaggcttcaacttcagagctttatggtttggttcaagaaattccacaaaaagagactacgccattttatccacgttcgccttatgctgttgcaaaattatatgcctattggatcactgttaattatcgtgagtcttatggtatgtttgcctgcaatggtattctctttaaccacgaatcacctcgccgtggcgagacctttgttactcgtaaaataacacgcgggatagcaaatattgctcaaggtcttgataaatgcttatacttgggaaatatggattctctgcgtgattggggacatgctaaggattatgtcaaaatgcaatggatgatgctgcagcaagaaactccagaagattttgtaattgctacaggaattcaatattctgtccgtgagtttgtcacaatggcggcagagcaagtaggcatagagttagcatttgaaggtgagggagtaaatgaaaaaggtgttgttgtttcggtcaatggcactgatgctaaagctgtaaacccgggcgatgtaattatatctgtagatccaaggtattttaggcctgcagaagttgaaaccttgcttggcgatcctactaatgcgcataaaaaattaggatggagccctgaaattacattgcgtgaaatggtaaaagaaatggtttccagcgatttagcaatagcgaaaaagaacgtcttgctgaaagctaataacattgccactaatattccgcaagaataaaaaagataatacattaaataattaaaaatggtgctagatttattagtaccattatttttttttgggtgactaatgtttattacatcagataaatttagagaaattatcaagttagttccattagtatcaattgatctgctaattgaaaacgagaatggtgaatatttatttggtcttaggaataatcgaccggccaaaaattatttttttgttccaggtggtaggattcgcaaaaatgaatctattaaaaatgcttttaaaagaatatcatctatggaattaggtaaagagtatggtatttcaggaagtgtttttaatggtgtatgggaacatttctatgatgatggttttttttctgaaggcgaggcaacacattatatagtgctttgttacacactgaaagttcttaaaagtgaattgaatctcccagatgatcaacatcgtgaatacctttggctaactaaacaccaaataaatgctaaacaagatgttcataactattcaaaaaattattttttgtaatttttattaaaaattaatatgcgagagaattgtatgtctcaatgtctttaccctgtaattattgccggaggaaccggaagccgtctatggccgttgtctcgagtattataccctaaacaatttttaaatttagttggggattctacaatgttgcaaacaacaattacgcgtttggatggcatcgaatgcgaaaatccaattgttatctgcaatgaagatcaccgatttattgtagcagagcaattacgacagattggtaagctaaccaagaatattatacttgagccgaaaggccgtaatactgcacctgccatagctttagctgcttttatcgctcagaagaataatcctaatgacgaccctttattattagtacttgcggcagaccactctataaataatgaaaaagcatttcgagagtcaataataaaagctatgccgtatgcaacttctgggaagttagtaacatttggaattattccggacacggcaaatactggttatggatatattaagagaagttcttcagctgatcctaataaagaattcccagcatataatgttgcggagtttgtagaaaaaccagatgttaaaacagcacaggaatatatttcgagtgggaattattactggaatagcggaatgtttttatttcgcgccagtaaatatcttgatgaactacggaaatttagaccagatatttatcatagctgtgaatgtgcaaccgctacagcaaatatagatatggactttgtccgaattaacgaggctgagtttattaattgtcctgaagagtctatcgattatgctgtgatggaaaaaacaaaagacgctgtagttcttccgatagatattggctggaatgacgtgggttcttggtcatcactttgggatataagccaaaaggattgccatggtaatgtgtgccatggggatgtgctcaatcatgatggagaaaatagttttatttactctgagtcaagtctggttgcgacagtcggagtaagtaatttagtaattgtccaaaccaaggatgctgtactggttgcggaccgtgataaagtccaaaatgttaaaaacatagttgacgatctaaaaaagagaaaacgtgctgaatactacatgcatcgtgcagtttttcgcccttggggtaaattcgatgcaatagaccaaggcgatagatatagagtaaaaaaaataatagttaaaccaggagaagggttagatttaaggatgcatcatcatagggcagagcattggattgttgtatccggtactgctaaagtttcactaggtagtgaagttaaactattagtttctaatgagtctatatatatccctcagggagcaaaatatagtcttgagaatccaggcgtaatacctttgcatctaattgaagtaagttctggtgattaccttgaatcagatgatatagtgcgttttactgacagatataacagtaaacaattcctaaagcgagattgataaatatgaataaaataacttgcttcaaagcatatgatatacgtgggcgtcttggtgctgaattgaatgatgaaatagcatatagaattggtcgcgcttatggtgagttttttaaacctcaaactgtagttgtgggaggagatgctcgcttaacaagtgagagtttaaagaaatcactctcaaatgggctatgtgatgcaggcgtaaatgtcttagatcttggaatgtgtggtactgaagagatatatttttccacttggtatttaggaattgatggtggaatcgaggtaactgcaagccataatccaattgattataatggaatgaaattagtaaccaaaggtgctcgaccaatcagcagtgacacaggtctcaaagatatacaacaattagtagagagtaataattttgaagagctcaacctagaaaaaaaagggaatattaccaaatattccacccgagatgcctacataaatcatttgatgggctatgctaatctgcaaaaaataaaaaaaatcaaaatagttgtgaattctgggaatggtgcagctggtcctgttattgatgctattgaggaatgctttttacggaacaatattccgattcagtttgtaaaaataaataatacacccgatggtaattttccacatggtatccctaatccattactacctgagtgcagagaagataccagcagtgcggttataagacatagtgctgattttggtattgcatttgatggtgattttgataggtgttttttctttgatgaaaatggacaatttattgaaggatactacattgttggtttattagcggaagtttttttagggaaatatccaaacgcaaaaatcattcatgatcctcgccttatatggaatactattgatatcgtagaaagtcatggtggtatacctataatgactaaaaccggtcatgcttacattaagcaaagaatgcgtgaagaggatgccgtatatggcggcgaaatgagtgcgcatcattattttaaagattttgcatactgcgatagtggaatgattccttggattttaatttgtgaacttttgagtctgacaaataaaaaattaggtgaactggtttgtggttgtataaacgactggccggcaagtggagaaataaactgtacactagacaatccgcaaaatgaaatagataaattatttaatcgttacaaagatagtgccttagctgttgattacactgatggattaactatggagttctctgattggcgttttaatgttagatgctcaaatacagaacctgtagtacgattgaatgtagaatctaggaataatgctattcttatgcaggaaaaaacagaagaaattctgaattttatatcaaaataaatttgcacctgagttcataatgggaacaagaaatatatgaaagtacttctgactggctcaactggcatggttggtaagaatatattagagcatgatagtgcaagtaaatataatatacttactccaaccagctctgatttgaatttattagataaaaatgaaatagaaaaattcatgcttatcaacatgccagactgtattatacatgcagcgggattagttggaggcattcatgcaaatataagcaggccgtttgattttctggaaaaaaatttgcagatgggtttaaatttagtttccgtcgcaaaaaaactaggtatcaagaaagtgcttaacttgggtagttcatgcatgtaccccaaaaactttgaagaggctattcctgagaaagctctgttaactggtgagctagaagaaactaatgagggatatgctattgcgaaaattgctgtagcaaaagcatgcgaatatatatcaagagaaaactctaattatttttataaaacaattatcccatgtaatttatatgggaaatatgataaatttgatgataactcgtcacatatgattccggcagttataaaaaaaatccatcatgcgaaaattaataatgtcccagagatcgaaatttggggggatggtaattcgcgccgtgagtttatgtatgcagaagatttagctgatcttattttttatgttattcctaaaatagaattcatgcctaatatggtaaatgctggtttaggttacgattattcaattaatgactattataagataattgcagaagaaattggttatactgggagtttttctcatgatttaacaaaaccaacaggaatgaaacggaagctagtagatatttcattgcttaataaaattggttggtcaagtcactttgaactcagagatggcatcagaaagacctataattattacttggagaatcaaaataaatgattacatacccacttgctagtaatacttgggatgaatatgagtatgcagcaatacagtcagtaattgactcaaaaatgtttaccatgggtaaaaaggttgagttatatgagaaaaattttgctgatttgtttggtagcaaatatgccgtaatggttagctctggttctacagctaatctgttaatgattgctgcccttttcttcactaataaaccaaaacttaaaagaggtgatgaaataatagtacctgcagtgtcatggtctacgacatattaccctctgcaacagtatggcttaaaggtgaagtttgtcgatatcaataaagaaactttaaatattgatatcgatagtttgaaaaatgctatttcagataaaacaaaagcaatattgacagtaaatttattaggtaatcctaatgattttgcaaaaataaatgagataataaataatagggatattatcttactagaagataactgtgagtcgatgggcgcggtctttcaaaataagcaggcaggcacattcggagttatgggtacctttagttctttttactctcatcatatagctacaatggaagggggctgcgtagttactgatgatgaagagctgtatcatgtattgttgtgccttcgagctcatggttggacaagaaatttaccaaaagagaatatggttacaggcactaagagtgatgatattttcgaagagtcgtttaagtttgttttaccaggatacaatgttcgcccacttgaaatgagtggtgctattgggatagagcaacttaaaaagttaccaggttttatatccaccagacgttccaatgcacaatattttgtagataaatttaaagatcatccattccttgatatacaaaaagaagttggtgaaagtagctggtttggtttttccttcgttataaaggagggagctgctattgagaggaagagtttagtaaataatctgatctcagcaggcattgaatgccgaccaattgttactgggaattttctcaaaaatgaacgtgttttgagttattttgattactctgtacatgatacggtagcaaatgccgaatatatagataagaatggtttttttgtcggaaaccaccagatacctttgtttaatgaaatagattatctacgaaaagtattaaaataactaacgaggcactctatttcgaatagagtgcctttaagatggtattaacagtgaaaaaaattttagcgtttggctattctaaagtactaccaccggttattgaacagtttgtcaatccaatttgcatcttcattatcacaccactaatactcaaccacctgggtaagcaaagctatggtaattggattttattaattactattgtatctttttctcagttaatatgtggaggatgttccgcatggattgcaaaaatcattgcagaacagagaattcttagtgatttatcaaaaaaaaatgctttacgtcaaatttcctataatttttcaattgttattatcgcatttgcggtattgatttcttttcttatattaagtatttgtttcttcgatgttgcgaggaataattcttcattcttattcgcgattattatttgtggtttttttcaggaagttgataatttatttagtggtgcgctaaaaggttttgaaaaatttaatgtatcatgtttttttgaagtaattacaagagtgctctgggcttctatagtaatatatggcatttacggaaatgcactcttatattttacatgtttagcctttaccattaaaggtatgctaaaatatattcttgtatgtctgaatattaccggttgtttcatcaatcctaattttaatagagttgggattgttaatttgttaaatgagtcaaaatggatgtttcttcaattaactggtggcgtctcacttagtttgtttgataggctcgtaataccattgattttatctgtcagtaaactggcttcttatgtcccttgccttcaactagctcaattgatgttcactctttctgcgtctgcaaatcaaatattactaccaatgtttgctagaatgaaagcatctaacacatttccctctaattgtttttttaaaattctgcttgtatcactaatttctgttttgccttgtcttgcgttattcttttttggtcgtgatatattatcaatatggataaaccctacatttgcaactgaaaattataaattaatgcaaattttagctataagttacattttattgtcaatgatgacatcttttcatttcttgttattaggaattggtaaatctaagcttgttgcaaatttaaatctggttgcagggctcgcacttgctgcttcaacgttaatcgcagctcattatggcctttatgcaatatctatggtaaaaataatatatccggcttttcaattttattacctttatgtagcttttgtctattttaatagagcgaaaaatgtctattgatttacttttttcaattactgaaatcgcaattgttttttcttgcactatttacatatttactcaatgtttgttaatgcggaggatctatttagataaaagtattttaattcttttatgcttgctcttttttttagtaatcattcaacttcctgagcttaatgtaaacggtttggtcgattctttaaagttatcactgcctttattgatggtctttatcgcttttcaaaaaccgaaattatgcttgtgggttattattgcattgttgtttttgaactctgcatttaattttttatatttaaagacattcgataagtttagctcatttccttttactttttttatattgctgttttacttgtttagattgggaattggtaatttaccggtttataaaaataaaaaattttacgcgttgatttttctctttatattaatagacataatgcagtcattgttaataaattatagggggcagattttatattccgtaatttgcatcctgatacttgtgtttaaagttaatttaagaaaaaagattccatacttttttttaatgctgccagttttatatgtaattattatggcttatattggttttaattatttcaataaaggcgtaactttttttgaacctacagcaagtaatattgaacgtacggggatgatatattatttggtttcacagcttggtgattatatattccatggtatggggacattaaatttcttaaataacggcggacaatataagacgttatatggacttccatcattaattcctaatgaccctcatgattttttattacggttctttataagtattggtgtgataggagcattggtttatcattctatattttttgttttttttaggagaatatctttcttattatatgagagaaatgctcctttcattgttgtaagttgtttgttactgttacaagttgtgttaatttatacattaaacccttttgatgcttttaatcgattgatttgcgggcttacagttggagttgtttatggatttgcaaaaattagataagtatacctgtaatggaaatttagacgctccacttgtttcaataatcattgcaacttataattctgaacttgatatagctaagtgtttgcaatcggtaactaatcaatcttataagaatattgaaatcataataatggatggaggatcttctgataaaacgcttgatattgcaaaatcgtttaaagacgaccgaataaaaatagtttcagagaaagatcgtggaatttatgatgcctggaataaagcagttgatttatccattggtgattgggtagcatttattggttcagatgatgtttactatcatacagatgcaattgcttcattgatgaagggggttatggtatctaatggcgcccctgtggtttatgggaggacagcgcacgaaggtcccgataggaacatatctggattttcaggcagtgaatggtacaacctaacaggatttaagtttaattattacaaatgtaatttaccattgcccattatgagcgcaatatattctcgtgatttcttcagaaacgaacgttttgatattaaattaaaaattgttgctgacgctgattggtttctgagatgtttcatcaaatggagtaaagagaagtcaccttattttattaatgacacgacccctattgttagaatgggatatggtggggtttcgactgatatttcttctcaagttaaaactacgctagaaagtttcattgtacgcaaaaagaataatatatcctgtttaaacatacagctgattcttagatatgctaaaattctggtgatggtagcgatcaaaaatatttttggcaataatgtttataaattaatgcataacgggtatcattccctaaagaaaatcaagaataaaatatgaagattgtttatataataaccgggcttacttgtggtggagccgaacaccttatgacgcagttagcagaccaaatgtttatacgcgggcatgatgttaatattatttgtctaactggtatatctgaggtaaagccaacacaaaatattaatattcattatgttaatatggataaaaattttagaagcttttttagagctttatttcaagtaaaaaaaataattgtcgccttaaagccagatataatacatagtcatatgtttcatgctaatatttttagtcgttttattaggatgctgattccagcggtgcccctgatatgtaccgcacacaacaaaaatgaaggtggcaatgcaaggatgttttgttatcgactgagtgattttttagcttctattactacaaatgtaagtaaagaggctgttcaagagtttatagcaagaaaggctacacctaaaaataaaatagtagagattccgaattttattaatacaaataaatttgattttgatattaatgtcagaaagaaaacgcgagatgcttttaatttgaaagacagtacagcagtactgctcgcagtaggaagacttgttgaagcaaaagactatccgaacttattaaatgcaataaatcatttgattctttcaaaaacatcaaattgtaatgattttattttgcttattgctggcgatggcgcattaagaaataaattattggatttggtttgtcaattgaatcttgtggataaagttttcttcttggggcaaagaagtgatattaaagaattaatgtgtgctgcagatctttttgttttgagttctgagtgggaaggttttggtctcgttgttgcagaagctatggcgtgtgaacgtcccgttgttgctaccgattctggtggagttaaagaagtcgttggacctcataatgatgttatccctgtcagtaatcatattctgttggcagagaaaatcgctgagacacttaaaatagatgataacgcaagaaaaataataggtatgaaaaatagagaatatattgtttccaatttttcaattaaaacgatagtgagtgagtgggagcgcttatattttaaatattccaagcgtaataatataattgattgaaaatataagtttgtactctggatgcaatagtttctctatgctgtttttttactggctccgtatttttacttatagctggattttgttatatatcagtattaatctgtctcaacttcatctagactacattcaagccgcgcatgcgtcgcgcggtgactacacctgacaggagtatgtaatgtccaagcaacagatcggcgtcgtcggtatggcagtgatggggcgcaacctggcgctcaacatcgaaagccgcggttataccgtctccatcttcaaccgctcccgcgagaaaactgaagaagttgttgccgagaacccggataagaaactggttccttattacacggtgaaagagttcgtcgagtctcttgaaaccccacgtcgtatcctgttaatggtaaaagcaggggcgggaactgatgctgctatcgattccctgaagccgtatctggataaaggcgacatcattattgatggtggcaacaccttcttccaggacactatccgtcgtaaccgtgaactgtccgcggaaggctttaacttcatcggtaccggcgtgtccggcggtgaagagggcgccctgaaaggcccatctatcatgccaggtggccagaaagaagcgtatgagctggttgcgcctatcctgaccaagattgctgcggttgctgaagatggcgaaccatgtataacttacatcggtgctgacggtgcgggtcactacgtgaagatggtgcacaacggtatcgaatatggcgatatgcagctgattgctgaagcctattctctgcttaaaggcggccttaatctgtctaacgaagagctggcaaccacttttaccgagtggaatgaaggcgagctaagtagctacctgattgacatcaccaaagacatcttcaccaaaaaagatgaagagggtaaatacctggttgatgtgatcctggacgaagctgcgaacaaaggcaccggtaaatggaccagccagagctctctggatctgggtgaaccgctgtcgctgatcaccgaatccgtattcgctcgctacatctcttctctgaaagaccagcgcattgcggcatctaaagtgctgtctggtccgcaggctaaactggctggtgataaagcagagttcgttgagaaagtccgtcgcgcgctgtacctgggtaaaatcgtctcttatgcccaaggcttctctcaactgcgtgccgcgtctgacgaatacaactgggatctgaactacggcgaaatcgcgaagatcttccgcgcgggctgcatcattcgtgcgcagttcctgcagaaaattactgacgcgtatgctgaaaacaaaggcattgctaacctgttgctggctccgtacttcaaaaatatcgctgatgaatatcagcaagcgctgcgtgatgtagtggcttatgctgtgcagaacggtattccggtaccgaccttctctgcagcggtagcctactacgacagctaccgttctgcggtactgccggctaatctgattcaggcacagcgtgattacttcggtgcgcacacgtataaacgcactgataaagaaggtgtgttccacaccg

SEQ ID NO: 7 - DNA, 20 bp, synthetic construct
  misc_feature 1..20: note = Description of sequence: Primer
  misc_feature 1..20: note = Name: PRIMER FOR O103-wbtDE-863F (forward)
  source 1..20: mol_type = other DNA
gtaaatggcgcgaagcaata

SEQ ID NO: 8 - DNA, 22 bp, synthetic construct
  misc_feature 1..22: note = Description of sequence: Primer
  misc_feature 1..22: note = Name: PRIMER FOR O103-wbtDE-1079R (reverse)
  source 1..22: mol_type = other DNA
ttgttggttaatgcatgactct

SEQ ID NO: 9 - DNA, 20 bp, synthetic construct
  misc_feature 1..20: note = Description of sequence: Primer
  misc_feature 1..20: note = Name: PRIMER FOR O121-vioA-210F (forward)
  source 1..20: mol_type = other DNA
tttgggtgttaaaggcgaag

SEQ ID NO: 10 - DNA, 20 bp, synthetic construct
  misc_feature 1..20: note = Description of sequence: Primer
  misc_feature 1..20: note = Name: PRIMER FOR O121-vioA-354R (reverse)
  source 1..20: mol_type = other DNA
cgcctcttcaattcttctcg

SEQ ID NO: 11 - DNA, 19 bp, synthetic construct
  misc_feature 1..19: note = Description of sequence: Probe
  misc_feature 1..19: note = Name: O26-Vir-Probe
  misc_feature 1: note = Modified_base - Modification: 6-FAM
  misc_feature 19: note = Modified_base - Modification: IABkFQ
  source 1..19: mol_type = other DNA
cagatattactgaaatacg

SEQ ID NO: 12 - DNA, 15 bp, synthetic construct
  misc_feature 1..15: note = Description of sequence: Probe
  misc_feature 1..15: note = Name: O26 AVir Probe
  misc_feature 1: note = Modified_base - Modification: HEX
  misc_feature 15: note = Modified_base - Modification: IABkFQ
  source 1..15: mol_type = other DNA
acagatattgctgaa

SEQ ID NO: 13 - DNA, 15 bp, synthetic construct
  misc_feature 1..15: note = Description of sequence: Probe
  misc_feature 1..15: note = Name: O111 AVir Probe
  misc_feature 1: note = Modified_base - Modification: HEX
  misc_feature 15: note = Modified_base - Modification: IABkFQ
  source 1..15: mol_type = other DNA
gttacaggcactaag

SEQ ID NO: 14 - DNA, 16 bp, synthetic construct
  misc_feature 1..16: note = Description of sequence: Probe
  misc_feature 1..16: note = Name: O111 Vir Probe
  misc_feature 1: note = Modified_base - Modification: 6-FAM
  misc_feature 16: note = Modified_base - Modification: IABkFQ
  source 1..16: mol_type = other DNA
ggttacaggtactaag

SEQ ID NO: 15 - DNA, 18 bp, synthetic construct
  misc_feature 1..18: note = Description of sequence: Probe
  misc_feature 1..18: note = Name: O103 AVir Probe
  misc_feature 1: note = Modified_base - Modification: HEX
  misc_feature 18: note = Modified_base - Modification: IABkFQ
  source 1..18: mol_type = other DNA
attaacctgcatattaaa

SEQ ID NO: 16 - DNA, 18 bp, synthetic construct
  misc_feature 1..18: note = Description of sequence: Probe
  misc_feature 1..18: note = Name: O103 Vir Probe
  misc_feature 1: note = Modified_base - Modification: 6-FAM
  misc_feature 18: note = Modified_base - Modification: IABkFQ
  source 1..18: mol_type = other DNA
attaacctgtatattaaa

SEQ ID NO: 17 - DNA, 19 bp, synthetic construct
  misc_feature 1..19: note = Description of sequence: Probe
  misc_feature 1..19: note = Name: O121 Vir Probe
  misc_feature 1: note = Modified_base - Modification: 6-FAM
  misc_feature 19: note = Modified_base - Modification: IABkFQ
  source 1..19: mol_type = other DNA
cgatattgattccaaaacc

SEQ ID NO: 18 - DNA, 19 bp, synthetic construct
  misc_feature 1..19: note = Description of sequence: Probe
  misc_feature 1..19: note = Name: O121 AVir Probe
  misc_feature 1: note = Modified_base - Modification: HEX
  misc_feature 18: note = Modified_base - Modification: IABkFQ
  source 1..19: mol_type = other DNA
cgatattgatcccaaaacc

SEQ ID NO: 19 - DNA, 19 bp, synthetic construct
  misc_feature 1..19: note = Description of sequence: Primer
  misc_feature 1..19: note = Name: 16S RNA-F
  source 1..19: mol_type = other DNA
cctcttgccatcggatgtg

SEQ ID NO: 20 - DNA, 21 bp, synthetic construct
  misc_feature 1..21: note = Description of sequence: Primer
  misc_feature 1..21: note = Name: 16S RNA-R
  source 1..21: mol_type = other DNA
ggctggtcatcctctcagacc

SEQ ID NO: 21 - DNA, 20 bp, synthetic construct
  misc_feature 1..20: note = Description of sequence: Probe
  misc_feature 1..20: note = Name: 16S rRNA Probe
  source 1..20: mol_type = other DNA
taacggctcacctaggcgac

SEQ ID NO: 22 - DNA, 20 bp, synthetic construct
  misc_feature 1..20: note = Description of sequence: Primer
  misc_feature 1..20: note = Name: Wzx O103-F
  source 1..20: mol_type = other DNA
ttggagcgttaactggacct

SEQ ID NO: 23 - DNA, 24 bp, synthetic construct
  misc_feature 1..24: note = Description of sequence: Primer
  misc_feature 1..24: note = Name: Wzx O103-R
  source 1..24: mol_type = other DNA
atattcgctatatcttcttgcggc

SEQ ID NO: 24 - DNA, 28 bp, synthetic construct
  misc_feature 1..28: note = Description of sequence: Primer
  misc_feature 1..28: note = Name: Wzx O103-P
  misc_feature 1: note = Modified_base - Modification: AE CAL Fluor Red 610
  misc_feature 28: note = Modified_base - Modification: BHQ2
  source 1..28: mol_type = other DNA
aggcttatctggctgttcttactacggc

SEQ ID NO: 25 - DNA, 20 bp, synthetic construct
  misc_feature 1..20: note = Description of sequence: Primer
  misc_feature 1..20: note = Name: O103
  misc_feature 1: note = Modified_base - Modification: H25-F
  source 1..20: mol_type = other DNA
gaaacaaacggtaaatggcg

SEQ ID NO: 26 - DNA, 21 bp, synthetic construct
  misc_feature 1..21: note = Description of sequence: Primer
  misc_feature 1..21: note = Name: O103
  misc_feature 1: note = Modified_base - Modification: H25-R
  source 1..21: mol_type = other DNA
tgttggttaatgcatgactct

SEQ ID NO: 27 - DNA, 21 bp, synthetic construct
  misc_feature 1..21: note = Description of sequence: Probe
  source 1..21: mol_type = other DNA
agaatattaacctgcatatta

SEQ ID NO: 28 - DNA, 21 bp, synthetic construct
  misc_feature 1..21: note = Description of sequence: Probe
  source 1..21: mol_type = other DNA
agaacattaacctgcatatta